(19) United States
(12) Patent Application Publication
(10) Pub. No.: US 2025/0051473 A1
(43) Pub. Date: Feb. 13, 2025

(54) ANTI-CD38 BINDING MOLECULES AND USES THEREOF

(71) Applicant: IGM Biosciences, Inc., Mountain View, CA (US)

(72) Inventors: Keyu LI, San Carlos, CA (US); Paul R. HINTON, Sunnyvale, CA (US); Ramesh BALIGA, Redwood City, CA (US); Bruce Alan KEYT, Hillsborough, CA (US)

(21) Appl. No.: 18/719,191

(22) PCT Filed: Feb. 3, 2023
(86) PCT No.: PCT/US2023/061932

§ 371 Date: Jun. 12, 2024

Related U.S. Application Data
(60) Provisional application No. 63/306,434, filed on Feb. 3, 2022.
(60) Provisional application No. 63/370,025, filed on Aug. 1, 2022.
(60) Provisional application No. 63/383,736, filed on Nov. 15, 2022.

(51) Int. Cl. C07K 16/28; A61K 39/00; A61P 35/00

(52) U.S. Cl. CPC C07K 16/2896; A61P 35/00; C07K 16/2809; A61K 2039/505; C07K 2317/24; C07K 2317/31; C07K 2317/73; C07K 2317/734; C07K 2317/92

(57) ABSTRACT

This disclosure provides an antibody or antigen-binding fragment or derivative thereof that specifically binds to CD38. Also provided are polynucleotides encoding the antibody or antigen-binding fragment or derivative thereof and vectors and host cell comprising said poly nucleotides. This disclosure further provides methods for producing and/or using an antibody or antigen-binding fragment or derivative thereof that specifically binds to CD38.

### CROSS-REFERENCE TO RELATED APPLICATIONS

This application claims the benefit of U.S. Provisional Patent Application Ser. Nos. 63/306,434, filed Feb. 3, 2022; 63/370,025, filed Aug. 1, 2022; and 63/383,736, filed Nov. 15, 2022, which are all each incorporated herein by reference in their entireties.

### SEQUENCE LISTING

The instant application contains a Sequence Listing which has been submitted electronically in ASCII format and is hereby incorporated by reference in its entirety. The ASCII copy, created on Feb. 3, 2023, is named 039WO1-Sequence-Listing and is 173,284 bytes in size.

### BACKGROUND

CD38 is expressed in various cancers, such as various hematologic malignancies including chronic lymphocytic leukemia (CLL), multiple myeloma (MM), Hodgkin's lymphoma (HL), diffuse large B-cell lymphoma (DLBCL), and peripheral T-cell lymphoma (PTCL), and various solid tumors, including prostate cancer, non-small cell lung cancer (NSCLC), squamous cell carcinoma of the head and neck (SCCHN), ovarian cancer, and liver cancer and is therefore considered a potential target for directed therapeutics (Martin et al., Cells, 2019, 8:1522).

Two anti-CD38 IgG antibodies, daratumumab and isatuximab, have been approved for the treatment of Multiple Myeloma (MM), the second most common blood cancer and representing 10% of all blood cancers. Despite the development these and other treatments, MM remains incurable with only a 40% 5-years survival rate. Most MM patients will become resistant to treatment (Marzo et al., Oncotarget, 2016, 7 (37): 60698-60711).

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 9,938,347, 10,570,191, 10,604,559, 10,618,978, 10,787,520, and 10,899,835, and U.S. Patent Application Publication Nos. 2019-0185570, 2019-0330360, 2019-0330374, 2019-0338041, and 2022-0106399, the contents of which are incorporated herein by reference in their entireties.

There remains a need to improve the treatment of CD38-expressing cancers, including MM.

### SUMMARY

Provided herein is an antibody or antigen-binding fragment or derivative thereof comprising an antigen-binding domain that specifically binds to CD38, wherein the antigen-binding domain comprises a heavy chain variable region (VH) and light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 182, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95. In some embodiments, the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

In some embodiments, the VH and VL comprise, respectively, an amino acid sequence at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142. In some embodiments, the VH and VL comprise, respectively, an amino acid sequence at least 80%, 85%, 90%, 95%, or 100% identical to the amino acid sequence of SEQ ID NO: 128 and SEQ ID NO: 133.

Also provided herein is an antibody or antigen-binding fragment or derivative thereof comprising an antigen-binding domain that specifically binds to CD38, wherein the antigen-binding domain comprises a heavy chain variable region (VH) and light chain variable region (VL), wherein the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161.

In some embodiments, the antibody or antigen-binding fragment or derivative thereof is a multimeric antibody comprising two, four, five, or six bivalent binding units and four, eight, ten, or twelve antigen-binding domains, wherein at least three, four, five, six, seven, eight, nine, ten, eleven, or twelve antigen-binding domains specifically bind to CD38; wherein each binding unit comprises two heavy chains each comprising an IgA or IgM constant region or a multimerizing fragment or variant thereof, and wherein at least three, four, five, six, seven, eight, nine, ten, eleven, or twelve heavy chain constant regions or fragments or variants thereof is/are associated with a copy of the VH.

In some embodiments, the antibody or antigen-binding fragment or derivative thereof is an Fv fragment, a single-chain Fv fragment (scFv), or a disulfide-linked Fv fragment (sdFv). In some embodiments, the antibody or antigen-binding fragment or derivative thereof comprises a complete antibody, an Fab fragment, an Fab′ fragment, or an F(ab′)₂ fragment.

In some embodiments, the antibody or antigen-binding fragment or derivative thereof comprises a bivalent binding unit comprising two antigen-binding domains, wherein at least one antigen-binding domain specifically binds to CD38, wherein the binding unit comprises two heavy chains each comprising a heavy chain constant region or fragment or variant thereof, and wherein at least one heavy chain constant region or fragment or variant thereof is associated with a copy of the VH.

In some embodiments, the heavy chains comprise IgG heavy chain constant regions or fragments or variants thereof fused to the C-terminus of the VH. In some embodiments, the binding unit further comprises two light chains each comprising a light chain constant region or fragment or variant thereof fused to the C-terminus of the VL.

Also provided herein is a multimeric antibody comprising two, four, five, or six bivalent binding units, wherein each binding unit comprises two IgM or IgA heavy chain constant regions or multimerizing fragments or variants thereof, each associated with an antigen-binding domain, wherein at least three, four, five, six, seven, eight, nine, ten, eleven, or twelve of the antigen-binding domains are a CD38 antigen binding domain comprising a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 182, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95. In some embodiments, the multimeric antibody is more potent than an equivalent amount of a bivalent IgG antibody comprising the VH and VL of the multimeric antibody. In some embodiments the multimeric can direct complement-directed cytotoxicity (CDC) of a CD38-expressing cell at a higher potency than an equivalent amount of a bivalent IgG antibody comprising the VH and VL of the multimeric antibody.

In some embodiments, the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71. In some embodiments, the VH and VL comprise, respectively, an amino acid sequence at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142. In some embodiments, the VH and VL comprise, respectively, an amino acid sequence at least 80%, 85%, 90%, 95%, or 100% identical to the amino acid sequence of SEQ ID NO: 128 and SEQ ID NO: 133.

Also provided herein is a multimeric antibody comprising two, four, five, or six bivalent binding units, wherein each binding unit comprises two IgM or IgA heavy chain constant regions or multimerizing fragments or variants thereof, each associated with an antigen-binding domain, wherein at least three, four, five, six, seven, eight, nine, ten, eleven, or twelve of the antigen-binding domains are a CD38 antigen binding domain comprising a heavy chain variable region (VH) and a light chain variable region (VL), wherein the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161.

In some embodiments, the multimeric antibody is more potent than an equivalent amount of a bivalent IgG antibody comprising the VH and VL of the multimeric antibody. In some embodiments the multimeric can direct complement-directed cytotoxicity (CDC) of a CD38-expressing cell at a higher potency than an equivalent amount of a bivalent IgG antibody comprising the VH and VL of the multimeric antibody.

In some embodiments, the multimeric antibody is dimeric or tetrameric and comprises two or four bivalent IgA or IgA-like binding units and a J chain or fragment or variant thereof, wherein each binding unit comprises two IgA heavy chain constant regions or multimerizing fragments or variants thereof each comprise a Cα3 domain and an α-tail piece (αtp) domain. In some embodiments, the IgA heavy chain constant regions or multimerizing fragments or variants thereof each further comprise a Cα1 domain, a Cα2 domain, an IgA hinge region, or any combination thereof. In some embodiments, the IgA heavy chain constant regions or multimerizing fragments or variants thereof are human IgA constant regions. In some embodiments, the J-chain is a mature human J-chain comprising the amino acid sequence SEQ ID NO: 7 or a functional fragment or variant thereof.

In some embodiments, the multimeric antibody is hexameric or pentameric and comprises five or six bivalent IgM or IgM-like binding units, wherein each binding unit comprises two IgM heavy chain constant regions or multimerizing fragments or variants thereof each comprise a Cμ4 and a μ-tail piece (μtp) domain. In some embodiments, the IgM heavy chain constant regions or fragments or variants thereof each further comprise a Cμ1 domain, a Cμ2 domain, a Cμ3 domain, or any combination thereof.

In some embodiments, the IgM heavy chain constant regions or multimerizing fragments or variants thereof are human IgM constant regions. In some embodiments, each IgM heavy chain constant region comprises the amino acid sequence SEQ ID NO: 1, SEQ ID NO: 2, or a multimerizing variant or fragment thereof.

In some embodiments, each IgM heavy chain constant region is a variant human IgM constant region comprising one or more single amino acid substitutions, deletions, or insertions relative to SEQ ID NO: 1 or SEQ ID NO: 2, and wherein the multimeric antibody has reduced CDC activity relative to a multimeric antibody where each IgM heavy chain constant region comprises the amino acid sequence SEQ ID NO: 1 or SEQ ID NO: 2. In some embodiments, each variant human IgM constant region comprises an amino acid substitution corresponding to position L310 of SEQ ID NO: 1 or SEQ ID NO: 2, an amino acid substitution corresponding to position P311 of SEQ ID NO: 1 or SEQ ID NO: 2, an amino acid substitution corresponding to position P313 of SEQ ID NO: 1 or SEQ ID NO: 2, an amino acid substitution corresponding to position K315 of SEQ ID NO: 1 or SEQ ID NO: 2, or any combination thereof.

In some embodiments, each IgM heavy chain constant region is a variant human IgM constant region comprising one or more single amino acid substitutions, deletions, or insertions relative to SEQ ID NO: 1 or SEQ ID NO: 2, and wherein the multimeric antibody exhibits increased serum half-life upon administration to a subject animal relative to a multimeric antibody where each IgM heavy chain constant region comprises the amino acid sequence SEQ ID NO: 1 or SEQ ID NO: 2, which is administered in the same way to the same animal species. In some embodiments, the variant IgM heavy chain constant regions comprise half-life altering amino acid substitutions at one or more amino acid positions corresponding to amino acid E345, S401, E402, or E403 of SEQ ID NO: 1 or SEQ ID NO: 2.

In some embodiments, the multimeric antibody is pentameric, and further comprises a J chain, or functional fragment thereof, or functional variant thereof.

In some embodiments, the J-chain is a mature human J-chain comprising the amino acid sequence SEQ ID NO: 7 or a functional fragment or variant thereof.

In some embodiments, the J-chain is a variant J-chain or functional fragment thereof comprising one or more single amino acid substitutions, deletions, or insertions relative to a reference J-chain identical to the variant J-chain except for the one or more single amino acid substitutions, deletions, or insertions, and wherein the variant J-chain can affect serum half-life of the multimeric antibody; and the multimeric antibody exhibits an increased serum half-life upon administration to a subject animal relative to a reference multimeric antibody that is identical except for the one or more single amino acid substitutions, deletions, or insertions in the variant J-chain, and is administered in the same way to the same animal species. In some embodiments, the variant J-chain comprises an amino acid substitution at an amino acid position corresponding to amino acid Y102 of the wild-type mature human J-chain of SEQ ID NO: 7. In some embodiments, the amino acid corresponding to Y102 of SEQ ID NO: 7 is substituted with alanine (A). In some embodiments, the J-chain comprises the amino acid sequence SEQ ID NO: 8.

In some embodiments, the J-chain or functional fragment or thereof is a modified J-chain further comprising a heterologous moiety, wherein the heterologous moiety is fused or conjugated to the J-chain or functional fragment or variant thereof. In some embodiments, the heterologous moiety is a polypeptide fused to the J-chain or functional fragment or variant thereof. In some embodiments, the heterologous polypeptide is fused to the J-chain or functional fragment or functional variant thereof via a peptide linker comprising at least 5 amino acids, but no more than 25 amino acids. In some embodiments, the heterologous polypeptide is fused to the N-terminus of the J-chain or functional fragment or variant thereof, to the C-terminus of the J-chain or functional fragment or variant thereof, or to both the N-terminus and C-terminus of the J-chain or functional fragment or variant thereof, wherein the heterologous polypeptides fused to both the N-terminus and C-terminus can be the same or different.

In some embodiments, the heterologous polypeptide comprises a scFv fragment. In some embodiments, the heterologous scFv fragment binds to CD3.

In some embodiments, the scFv fragment comprises a scFv heavy chain variable region (scFv VH) and a scFv light chain variable region (scFv VL), wherein the scFv VH comprises scFv VH complementarity-determining regions VHCDR1, VHCDR2, and VHCDR3 and the scFv VL comprises scFv VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49. In some embodiments, the VHCDR1, VHCDR2, VHCDR3, VLCDR1, VLCDR2, and VLCDR3 comprise, respectively, SEQ ID NO: 25, SEQ ID NO: 26, SEQ ID NO: 27, SEQ ID NO: 29, SEQ ID NO: 30, and SEQ ID NO: 31. In some embodiments, the scFv VH and scFv VL comprise an amino acid sequence at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively. In some embodiments, the scFv fragment comprises the scFv VH and scFv VL amino acid sequences SEQ ID NO: 24 and SEQ ID NO: 28, respectively.

In some embodiments, the antibody or antigen-binding fragment or derivative thereof or the multimeric antibody can specifically bind to human CD38, non-human primate CD38, or human CD38 and non-human primate CD38. In some embodiments, the non-human primate CD38 is cynomolgus monkey CD38.

In some embodiments, the antibody or antigen-binding fragment or derivative thereof or the multimeric antibody specifically binds to CD38 with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM. 0.005 nM, or 0.001 nM; and wherein the CD38 is human CD38, cynomolgus monkey CD38, or human CD38 and cynomolgus monkey CD38.

In some embodiments, the antibody or antigen-binding fragment or derivative thereof or the multimeric antibody is multispecific. In some embodiments, the antibody or antigen-binding fragment or derivative thereof or the multimeric antibody is bispecific.

Also provided herein is a composition comprising the antibody or antigen-binding fragment or derivative thereof or the multimeric antibody.

Also provided herein is a polynucleotide comprising a nucleic acid sequence that encodes the antibody or antigen-binding fragment or derivative thereof or the multimeric antibody. Also provided herein is a vector comprising the polynucleotide. Also provided herein is a host cell comprising the vector. Also provided herein is a method of producing the antibody or antigen-binding fragment or derivative thereof or the multimeric antibody comprising culturing the host cell and recovering the antibody or antigen-binding fragment or derivative thereof.

Also provided herein is a method of treating cancer comprising administering to a subject in need of treatment an effective amount of the antibody or antigen-binding fragment or derivative thereof or the multimeric antibody. In some embodiments, the subject is human.

In some embodiments, the antibody or antigen-binding fragment or derivative thereof, the multimeric antibody, or the composition is for use in treating cancer in a subject in need thereof. In some embodiments, the subject is human.

Also provided herein is a use of the antibody or antigen-binding fragment or derivative thereof of or the multimeric antibody in treating cancer in a subject in need thereof. Also provided herein is a use of the antibody or antigen-binding fragment or derivative thereof or the multimeric antibody in the manufacture of a medicament for treating cancer in a subject in need thereof. In some embodiments, the subject is human.

### BRIEF DESCRIPTION OF THE DRAWINGS/FIGURES

FIG. 1A-1B shows binding of chimeric CD38×CD3 IgM antibodies to human CD38 (FIG. 1A) and cynomolgus monkey (“cyno”) CD38 (FIG. 1B). Data is presented in relative luminescence units (RLU) for the binding of chimeric CD38×CD3 IgM antibodies (IgM A-1, IgM B-1, IgM C-1, IgM E-1, IgM F-1, and IgM H-1).

FIG. 2 shows binding of chimeric CD38×CD3 IgM antibodies to NCI-H929 cells measured by flow cytometry. Data is presented as median fluorescence intensity for binding of chimeric CD38×CD3 IgM antibodies (IgM A-1, IgM B-1, IgM C-1, IgM E-1, IgM F-1, and IgM H-1).

FIG. 3 shows binding of humanized CD38×CD3 IgM antibodies to human CD38 at different antibody concentrations measured by ELISA. Data is presented in relative luminescence units (RLU) for humanized CD38×CD3 IgM antibodies (IgM B-2, IgM B-4, IgM E-2, IgM E-4, IgM OKT10-2, and IgM OKT10-4).

FIG. 4 shows the pharmacokinetics of anti-CD38×CD3 IgM antibodies (IgM B-2, IgM E-2, and IgM OKT10-2) in a mouse model.

FIG. 5A shows the dose response curve for killing of H929 cells (Killing %) with increasing concentration of CD38×CD3 IgG and IgM B-2. FIG. 5B shows TNF-α levels that resulted following treatment with increasing concentration of CD38×CD3 IgG and IgM B-2. FIG. 5C shows IFN-γ levels that resulted following treatment with increasing concentration of CD38×CD3 IgG and IgM B-2.

FIG. 6A shows CD8+ T cell count for each PBMC donor, following CD38×CD3 IgG or IgM B-2 treatment. FIG. 6B shows the ratio of CD8+ T cell count to regulatory T cell count (Treg) for each PBMC donor, following CD38×CD3 IgG or IgM B-2 treatment.

FIG. 6C shows CD38hi regulatory T cell count for each PBMC donor, following CD38×CD3 IgG or IgM B-2 treatment.

FIG. 7A shows in vitro colony formation of multiple myeloma cells from four different donors, following treatment with a solvent control, 10 nM IgM B-2, or 50 nM IgM B-2.

FIG. 7B shows in vitro colony formation of erythroid cells from two different normal bone marrow donors, following treatment with a solvent control, 10 nM IgM B-2, or 50 nM IgM B-2. Total erythroid cells were measured at the CFU-E (colony forming unit-erythroid) and BFU-E (erythroid burst-forming unit) stages of development.

FIG. 7C shows in vitro colony formation of granulocyte-macrophage progenitors (CFU-GM) from two different normal bone marrow donors, following treatment with a solvent control, 10 nM IgM B-2 or 50 nM IgM B-2.

FIG. 8 shows T cell fratricide levels for treatment with IgM B-2 or CD38×CD3 IgG for CD4+ and CD8+ T cells.

FIG. 9A and FIG. 9B both show the median BLI signal (tumor burden) over time in two separate trials of animals dosed with vehicle, CD38×CD3 IgG, or IgM B-2.

FIG. 10A-10F shows the BLI signal (tumor burden) over time for each of the animals treated with vehicle (FIG. 10A), 0.5 mg/kg CD38×CD3 IgG (FIG. 10B), 0.1 mg/kg CD38×CD3 IgG (FIG. 10C), 10 mg/kg IgM B-2 (FIG. 10D), 3 mg/kg IgM B-2 (FIG. 10E), or 1 mg/kg IgM B-2 (FIG. 10F).

FIG. 11A-11B shows the number of CD4+ T cells (FIG. 11A) and CD8+ T cells (FIG. 11B) per microliter of blood in mice treated with vehicle, 0.5 or 0.1 mg/kg CD38×CD3 IgG, or 10, 3, or 1 mg/kg IgM B-2.

FIG. 12A-12B shows the number of CD4+ T cells (FIG. 12A) and CD8+ T cells (FIG. 12B) per bone marrow sample in mice treated with vehicle, 0.5 mg/kg CD38×CD3 IgG, or 3 mg/kg IgM B-2.

FIG. 13A-13B shows the number of CD4+ T cells (FIG. 13A) and CD8+ T cells (FIG. 13B) per mg of spleen sample in mice treated with vehicle, 0.5 mg/kg CD38×CD3 IgG, or 3 mg/kg IgM B-2.

FIG. 14A shows the ratio of CD8+ T cells to regulatory T cells (Treg) (CD8+T: Treg) in peripheral blood of mice treated with vehicle, 0.1 or 0.5 mg/kg CD38×CD3 IgG or 1, 3, or 10 mg/kg IgM B-2.

FIG. 14B shows the ratio of CD8+ T cells to regulatory T cells (Treg) (CD8+T: Treg) in bone marrow of mice treated with vehicle, 0.5 mg/kg CD38×CD3 IgG or 3 mg/kg IgM B-2.

FIG. 14C shows the ratio of CD8+ T cells to regulatory T cells (Treg) (CD8+T: Treg) in spleen of mice treated with vehicle, 0.5 mg/kg CD38×CD3 IgG, or 3 mg/kg IgM B-2.

FIG. 15 shows the dose response curves for complement-mediated cytotoxicity (killing %) of various cell lines with increasing concentrations of IgM B-2, CD38 IgG #Y, CD38 IgG #Z, and an isotype control IgM antibody. FIG. 15A: MOLP-8 cells, FIG. 15B: LP-1 cells, FIG. 15C: Ramos cells, FIG. 15D: Raji cells, FIG. 15E: RPMI-8226 cells, FIG. 15F: NCI-H929 cells, FIG. 15G: OPM-2 cells, FIG. 15H: MOLM-13 cells.

FIG. 16A shows viability of primary NK cells as a function of antibody concentration. FIG. 16B shows viability of primary monocytes as a function of antibody concentration.

FIG. 17 shows BLI signal in a disseminated NCI-H929-luc-gfp-Puro tumor model in NSG-MHC I/II DKO mice humanized with PBMCs and treated with IgM B-2 (10 mg/kg) three times per week for 11 doses. Error bars represent mean±SEM of 9 animals per group. Statistical analyses represent comparisons to vehicle control group. Each vertical line indicates a day of antibody dosing.

FIG. 18A shows tumor volume over time as a function of IGM B-2 dose in a humanized mouse NCI-H929 tumor xenograft model. Error bars represent mean±SEM of 9 animals per group. Statistical analyses represent comparisons to vehicle control group. Treatment was administered three times each week for a total of 15 doses. Each triangle above indicates day of antibody dosing. FIG. 18B shows percent body weight change following IGM B-2 treatment. FIG. 18C shows details of the D₂₁ results from panel A. P values obtained upon comparing treatment groups to vehicle group, ** p=0.0049 (30 mg/kg), ** p=0.0045 (10 mg/kg), p=0.15 (3 mg/kg) and p=0.18 (1 mg/kg). FIG. 18D shows overall survival in the humanized mouse NCI-H929 tumor xenograft model.

FIG. 19A shows tumor volume over time as a function of IGM B-2 dose in a humanized Raji mouse tumor xenograft model. Error bars represent mean±SEM of 9 animals per group. Statistical analyses represent comparisons to vehicle control group. Treatment was administered three times each week for a total of 15 doses. Each triangle above indicates day of antibody dosing. FIG. 19B shows details of the D₂₅ results from panel A. P values obtained upon comparing treatment groups to vehicle group, p<0.0001 (30 mg/kg), *** p=0.0008 (10 mg/kg), p=0.12 (3 mg/kg) and p=0.33 (1 mg/kg).

FIG. 19C shows percent body weight change following IGM B-2 treatment. FIG. 19D shows overall survival in the humanized Raji xenograft mouse tumor xenograft model.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the polypeptide or antibody binds. Methods of identifying nucleotide and amino acid conservative substitutions which do not eliminate antigen-binding are well-known in the art (see, e.g., Brummell et al., Biochem. 32:1180-1 187 (1993); Kobayashi et al., Protein Eng. 12 (10): 879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds to a binding target, e.g., an epitope or an antigenic determinant. As described further herein, a binding molecule can comprise one of more “antigen-binding domains” described herein. A non-limiting example of a binding molecule is an antibody or antibody-like molecule that retains antigen-specific binding, or an antibody-like molecule or fragment thereof as described in detail herein that retains antigen-specific binding. In certain embodiments a “binding molecule” comprises an antibody or antibody-like molecule as described in detail herein.

As used herein, the terms “binding domain” or “antigen-binding domain” (can be used interchangeably) refer to a region or fragment of a binding molecule e.g., an antibody or antibody-like molecule, that is necessary and sufficient to specifically bind to a binding target, e.g., an epitope. For example, an “Fv,” e.g., a heavy chain variable region and a light chain variable region of an antibody, either as two separate polypeptide subunits or as a single chain, is considered to be a “binding domain.” Other antigen-binding domains include, without limitation, the heavy chain variable region (VHH) of an antibody derived from a camelid species, or six immunoglobulin complementarity determining regions (CDRs) expressed in a scaffold, e.g., a fibronectin scaffold. A “binding molecule,” e.g., an antibody or antibody-like molecule as described herein can include one, two, three, four, five, six, seven, eight, nine, ten, eleven, twelve, or even more “antigen-binding domains.” As used herein, a “binding unit-associated antigen-binding domain” refers to an antigen binding domain that is part of an antibody heavy chain and/or an antibody light chain. The term “J-chain-associated antigen-binding domain” refers to an antigen binding domain that is associated with a modified J-chain as described herein, for example, a scFv fused to a wild-type human J-chain, or functional fragment or variant thereof.

The terms “antibody” and “immunoglobulin” can be used interchangeably herein. An antibody as provided in this disclosure must specifically bind to an antigen, i.e., it includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two IgG heavy chains or fragments thereof and two light chains, an IgA antibody that includes two, four, or eight IgA heavy chains or multimerizing fragments thereof and two, four, or eight light chains and optionally includes a J-chain or functional fragment or variant thereof and/or a secretory component, or an IgM antibody that includes ten or twelve IgM heavy chains or multimerizing fragments thereof and ten or twelve light chains and optionally includes a J-chain or functional fragment or variant thereof.

The term “immunoglobulin” comprises various broad classes of polypeptides that can be distinguished biochemically. Those skilled in the art will appreciate that heavy chains are classified as, e.g., gamma, mu, alpha, delta, or epsilon, (γ, μ, α, δ, ε) with some subclasses among them (e.g., γ1-γ4 or α1-α2). It is the nature of this chain that determines the “isotype” of the antibody as IgG, IgM, IgA, IgD, or IgE, respectively. The immunoglobulin subclasses (subtypes) e.g., IgG1, IgG2, IgG3, IgG4, IgA1, IgA2, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (K, 2). Each heavy chain class can be associated with either a kappa or lambda light chain. In general, the light and heavy chains are covalently bonded to each other via disulfide bonds, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody or antibody-like molecule that corresponds to a standard “H2L2” immunoglobulin structure, e.g., two heavy chains or fragments thereof and two light chains or fragments thereof. In certain embodiments a binding unit can correspond to two heavy chains, e.g., in a camelid antibody. In certain embodiments, e.g., where the binding molecule is a bivalent IgG antibody or antibody-like molecule the terms “binding molecule” and “binding unit” are equivalent. In other embodiments, e.g., where the binding molecule is multimeric, e.g., a dimeric or tetrameric IgA antibody or IgA-like antibody, a pentameric IgM antibody or IgM-like antibody, or a hexameric IgM antibody or IgM-like antibody, the binding molecule comprises two or more “binding units.” Two or four in the case of an IgA dimer or tetramer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric” or “tetrameric,” and include two or four bivalent binding units that include IgA heavy chain constant regions or multimerizing fragments thereof. Certain binding molecules provided in this disclosure are “pentameric” or “hexameric,” and include five or six bivalent binding units that include IgM heavy chain constant regions or multimerizing fragments thereof. A binding molecule, e.g., an antibody or antibody-like molecule comprising two or more, e.g., two, four, five, or six binding units, is referred to herein as “multimeric.”

The term “J-chain” as used herein refers to the J-chain associated with pentameric IgM or dimeric or tetrameric IgA antibodies of any animal species, any functional fragment thereof, derivative thereof, and/or variant thereof, including the mature human J-chain, the amino acid sequence of which is presented as SEQ ID NO: 7. Various J-chain variants and modified J-chain derivatives are disclosed herein. As persons of ordinary skill in the art will recognize, “a functional fragment” or a “functional variant” includes those fragments and variants that can associate with IgM heavy chain constant regions to form a pentameric IgM antibody (or alternatively can associate with IgA heavy chain constant regions to form a dimeric or tetrameric IgA antibody).

The term “modified J-chain” is used herein to refer to a derivative of a J-chain polypeptide comprising a heterologous moiety, e.g., a heterologous polypeptide, e.g., an extraneous binding domain introduced into the J-chain polypeptide. The introduction can be achieved by any means, including direct or indirect fusion of the heterologous polypeptide or other moiety or by attachment through a peptide or chemical linker. The term “modified human J-chain” encompasses, without limitation, a human J-chain comprising the amino acid sequence of SEQ ID NO: 7 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a dimer or tetramer, and binding of such polymers to a target. Exemplary modified J-chains can be found, e.g., in U.S. Pat. Nos. 9,951,134, 10,975,147, 10,400,038, and 10,618,978, and in U.S. Patent Application Publication No. US-2019-0185570, each of which is incorporated herein by reference in its entirety.

As used herein, the terms “IgM-derived binding molecule,” “IgM-like antibody,” “IgM-like binding unit,” or “IgM-like heavy chain constant region” refer to a variant antibody-derived binding molecule, antibody, binding unit, or heavy chain constant region that still retains the structural portions of an IgM heavy chain necessary to confer the ability to bind to antigen and to form multimers, i.e., hexamers, or in association with J-chain, form pentamers. An IgM-like antibody or IgM-derived binding molecule typically includes at least the Cμ4 and μ tailpiece (μtp) domains of the IgM constant region and an antigen binding domain or subunit thereof but can include heavy chain constant region domains from other antibody isotypes, e.g., IgG, from the same species or from a different species. An IgM-like antibody or IgM-derived binding molecule can likewise be an antibody fragment in which one or more constant regions are deleted, as long as the IgM-like antibody is capable of binding antigen and of forming hexamers and/or pentamers. Thus, an IgM-like antibody or IgM-derived binding molecule can be, e.g., a hybrid IgM/IgG antibody or can be a “multimerizing fragment” of an IgM antibody.

As used herein, the terms “IgA-derived binding molecule,” “IgA-like antibody,” “IgA-like binding unit,” or “IgA-like heavy chain constant region” refer to a variant antibody-derived binding molecule, antibody, binding unit, or heavy chain constant region that still retains the structural portions of an IgA heavy chain necessary to bind antigen and to confer the ability to form multimers, i.e., dimers or tetramers, in association with J-chain. An IgA-like antibody or IgA-derived binding molecule typically includes at least the Cα3 and a tailpiece (μtp) domains of the IgA constant region and an antigen binding domain or subunit thereof but can include heavy chain constant region domains from other antibody isotypes, e.g., IgG, from the same species or from a different species. An IgA-like antibody or IgA-derived binding molecule can likewise be an antibody fragment in which one or more constant regions are deleted, as long as the IgA-like antibody is capable of binding antigen and forming dimers in association with a J-chain. Thus, an IgA-like antibody or IgA-derived binding molecule can be, e.g., a hybrid IgA/IgG antibody or can be a “multimerizing fragment” of an IgA antibody.

The terms “valency,” “monovalent,” “bivalent,” “multivalent” and grammatical equivalents, refer to the number of antigen-binding domains in given binding molecule, e.g., an antibody or antibody-like molecule or in a given binding unit. As such, the terms “bivalent,” “tetravalent,” and “hexavalent” in reference to a given binding molecule, e.g., an IgM antibody, IgM-like antibody or multimerizing fragment thereof, denote the presence of two antigen-binding domains, four antigen-binding domains, and six antigen-binding domains, respectively. A typical IgM antibody or IgM-like antibody or IgM-derived binding molecule where each binding unit is bivalent can have 10 or 12 valencies. A bivalent or multivalent binding molecule, e.g., antibody or antibody-like molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody or antibody-like molecule. In certain embodiments, an epitope can include chemically active surface groupings of molecules such as amino acids, sugar side chains, phosphoryl, or sulfonyl, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., an antibody or antibody-like molecule. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, e.g., an antibody or antibody-like molecule.

Both the light and heavy chains of an antibody or antibody-like molecule are divided into regions of structural and functional homology. The terms “constant” and “variable” are used functionally but refer to particular structures of the molecule. The variable regions of both the light (VL) and heavy (VH) chains determine antigen recognition and specificity. Conversely, the constant domains of the light chain (CL) and the heavy chain (e.g., CH1, CH2, CH3, or CH4) confer biological properties such as the ability to multimerize, secretion, transplacental mobility, Fc receptor binding, complement binding, and the like. By convention the numbering of the constant region domains increases as they become more distal from the antigen-binding regions or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a μ tailpiece.

As indicated above, variable region(s) form the antigen-binding domain of the antibody or antibody-like molecule, allowing it to selectively recognize and specifically bind epitopes on antigens. That is, the VL domain and VH domain, or an antigen-binding subset of the complementarity determining regions (CDRs), of a binding molecule, e.g., an antibody or antibody-like molecule combine to form the antigen-binding domain. More specifically, an antigen-binding domain can be defined by three CDRs on each of the VH and VL chains. Certain antibodies or antibody-like molecules form larger structures. For example, IgA heavy chains can form a molecule that includes two or four H2L2 binding units and a J-chain covalently connected via disulfide bonds, which can be further associated with a secretory component, and IgM heavy chains can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the target antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, e.g., “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res., 36: W503-508, 2008).

Kabat et al. also defined a numbering system for variable region and constant region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine(S) (SEQ ID NO: 1) or glycine (G) (SEQ ID NO: 2)):

Sequential (SEQ ID NO: 1 or SEQ ID NO: 2)/KABAT numbering key for IgM heavy chain

```
    1/127 GSASAPTLFP LVSCENSPSD TSSVAVGCLA
                QDELPDSITE SWKYKNNSDI
  51/176 SSTRGFPSVL RGGKYAATSQ VLLPSKDVMQ
                GTDEHVVCKV QHPNGNKEKN
101/226 VPLPVIAELP PKVSVFVPPR DGFFGNPRKS
                KLICQATGFS PRQIQVSWLR
151/274 EGKQVGSGVT TDQVQAEAKE SGPTTYKVTS
                TLTIKESDWL XQSMFTCRVD
201/324 HRGLTFQQNA SSMCVPDQDT AIRVFAIPPS
                FASIFLTKST KLTCLVTDLT
251/374 TYDSVTISWT RQNGEAVKTH TNISESHPNA
                TESAVGEASI CEDDWNSGER
301/424 FTCTVTHTDL PSPLKQTISR PKGVALHRPD
                VYLLPPAREQ LNLRESATIT
351/474 CLVTGFSPAD VEVQWMQRGQ PLSPEKYVTS
                APMPEPQAPG RYFAHSILTV
401/524 SEEEWNTGET YTCVVAHEAL PNRVTERTVD
                KSTGKPTLYN VSLVMSDTAG
451/574 TCY
```

Binding molecules, e.g., antibodies, antibody-like molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are described, e.g., in U.S. Pat. No. 5,892,019.

By “specifically binds,” it is generally meant that a binding molecule, e.g., an antibody or antibody-like molecule binds to an epitope via its antigen-binding domain, and that the binding entails some complementarity between the antigen-binding domain and the epitope. According to this definition, a binding molecule, e.g., an antibody or antibody-like molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k (off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, 10⁻⁵ sec⁻¹, 5×10⁻⁶ sec⁻¹, 10⁻⁶ sec⁻¹, 5×10⁻⁷ sec⁻¹, or 10⁻⁷ sec⁻¹.

A binding molecule, e.g., an antibody or antibody-like molecule disclosed herein can be said to bind a target antigen with an on rate (k (on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, 5×10⁶ M⁻¹ sec⁻¹ or 10⁷ M⁻¹ sec⁻¹.

A binding molecule, e.g., an antibody or antibody-like molecule is said to competitively inhibit binding of a reference antibody or antibody-like molecule to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays or OCTET assays. A binding molecule can be said to competitively inhibit binding of the reference antibody or antibody-like molecule to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an antibody or antibody-like molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and the valencies of the immunoglobulins and the antigen. For example, the interaction between a bivalent monoclonal antibody and an antigen with a highly repeating epitope structure, such as a polymer, would be one of high avidity. Likewise, the interaction between a multimeric antibody with four, eight, ten, or twelve valencies and a population of specific epitopes would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or KD no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antigen-binding fragments of a binding molecule or antibody as provided herein, including single-chain antibodies or other antigen-binding domains that can exist alone or in combination with one or more of the following: hinge region, CH1, CH2, CH3, or CH4 domains, J-chain, or secretory component. Also included are antigen-binding fragments that can include any combination of variable region(s) sufficient to bind antigen with one or more of a hinge region, CH1, CH2, CH3, or CH4 domains, a J-chain, or a secretory component. Binding molecules, e.g., antibodies or antibody-like molecules can be from any animal origin including birds and mammals. The antibodies can be, e.g., human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM or IgM-like antibody or IgM-derived binding molecule as provided herein can include an antigen-binding fragment of an antibody, e.g., a scFv, so long as the IgM or IgM-like antibody is able to form a multimer, e.g., a hexamer or a pentamer.

As used herein, the term “heavy chain subunit” includes amino acid sequences derived from an immunoglobulin heavy chain. A binding molecule, e.g., an antibody or antibody-like molecule comprising a heavy chain subunit can include a VH domain and one or more of a CH1 domain, a hinge (e.g., upper, middle, and/or lower hinge region) domain, a CH2 domain, a CH3 domain, a CH4 domain, a μ tail-piece (μtp), or a variant or fragment thereof. For example, a binding molecule, e.g., an antibody, antibody-like molecule, or fragment, variant, or derivative thereof can include without limitation, in addition to a VH domain, any combination of a CH1 domain, a hinge, a CH2 domain; a CH3 domain; a CH4 domain; or a μ tailpiece (μtp) of one or more antibody isotypes and/or species. In certain embodiments, a binding molecule, e.g., an antibody, antibody-like molecule, or fragment, variant, or derivative thereof can include, in addition to a VH domain, one or more of a CH1 domain, a CH2 domain, a CH3 domain, a CH4 domain, a μ-tailpiece (μtp) domain and a J-chain (in the case of IgM), or one or more of a CH1 domain, a hinge region, a CH2 domain, a CH3 domain, an α-tailpiece (αtp) domain, and a J-chain (in the case of IgA). Further, a binding molecule, e.g., antibody or antibody-like molecule provided in the disclosure can lack certain constant region portions, e.g., all or part of a CH1 domain, a hinge, a CH2 domain, or a CH3 domain. These domains (e.g., the heavy chain subunit) can be modified such that they vary in amino acid sequence from the original immunoglobulin molecule. According to embodiments of the present disclosure, an IgM or IgM-like antibody as provided herein includes sufficient portions of an IgM heavy chain constant region to allow the IgM or IgM-like antibody to form a multimer, e.g., a hexamer or a pentamer, e.g., the IgM heavy chain constant region includes a “multimerizing fragment” of an IgM heavy chain constant region.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antigen-binding fragments, variants, or derivatives thereof, or multimerizing fragments thereof can be described or specified in terms of the epitope(s) or portion(s) of an antigen that they recognize or specifically bind. The portion of a target antigen that specifically interacts with the antigen-binding domain of an antibody is an “epitope,” or an “antigenic determinant.” A target antigen can comprise a single epitope or two or more epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen-binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody or antibody-like molecule that has antigen-binding domains for two or more different epitopes within a single antibody molecule. Other binding molecules in addition to the canonical antibody structure can be constructed with two binding specificities.

As used herein, the term “engineered antibody” refers to an antibody in which the variable domain in either the heavy and light chain or both is altered by at least partial replacement of one or more amino acids in either the CDR or framework regions. In certain embodiments, entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments, not all the CDRs are replaced with the complete CDRs from the donor variable region and yet the antigen-binding capacity of the donor can still be transferred to the recipient variable domains. Exemplary methods of humanization are described in U.S. Pat. Nos. 5,585,089, 5,693,761, 5,693,762, and 6,180,370.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide. The term “associated” and grammatical equivalents refers to the interaction of two or more elements function together and that can be linked or fused, but can also be in proximity, e.g., interacting in trans without being connected in any particular way.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed disease, pathologic condition, or disorder. Terms such as “prevent,” “prevention,” “avoid,” “deterrence” and the like refer to prophylactic or preventative measures that prevent the development of an undiagnosed targeted disease, pathologic condition, or disorder. Thus, “a subject need of treatment” can include those already with the disease, pathologic condition. The term “a subject in need of prevention” those subjects prone to have the disease, pathologic condition, or disorder and those in whom the disease, pathologic condition or disorder is to be prevented.

As used herein, the “potency” of a therapeutic agent, e.g., a binding molecule, is a measure of the agent's activity expressed in terms of the amount required to produce a desired biological effect, either in vitro or in vivo. Potency can be expressed, e.g., as the concentration or amount of a given therapeutic agent required to affect, e.g., kill, 50% of test cells in an in vitro assay, e.g., a complement dependent cytotoxicity (CDC) or T cell dependent cytotoxicity assay (TDCC) assay. This measurement can be expressed as the 50% effective concentration (EC50) or 50% inhibitory concentration (IC50). Potency can also be expressed as a curve in which % survival or % killing of test cells is on the Y axis, and binding molecule concentration (in, e.g., μg/ml or μM) is on the X axis. “Potency” can also define the ability of a given therapeutic agent to treat a disease or condition in a subject, e.g., an in vivo test animal or a human patient. “Potency” is often used to compare various different therapeutic agents in a given assay or experiment, e.g., to show the superiority of a certain therapeutic agent over other therapeutic agents with a similar or identical mechanism of action. Effector cell engaging molecules, e.g., T cell engaging molecules as provided herein can have one or more tumor targeting binding domains, e.g., CD38 binding domains and one or more effector cell engaging binding domains, e.g., a CD3 binding domain, and therefore the molecule as a whole can have at least two potency measurements-related to the ability of the molecule to bind to and kill tumor cells, e.g., through antibody-dependent cellular cytotoxicity (ADCC) or complement-dependent cytotoxicity (CDC), and related to the ability of the molecule to engage and activate effector cells, e.g., through T cell dependent cytotoxicity (TDCC).

As used herein the terms “serum half-life” or “plasma half-life” refer to the time it takes (e.g., in minutes, hours, or days) following administration for the serum or plasma concentration of a protein or a drug, e.g., a binding molecule such as an antibody or antibody-like molecule as described herein, to be reduced by 50%. Two half-lives can be described: the alpha half-life, a half-life, or tiza, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t₀) to infinity (0) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

### CD38 Target

CD38, also known as ADP-ribosyl cyclase/cyclic ADP-ribose hydrolase 1, is a single-pass type II membrane protein. It has several known functions, including as an ADP-ribosyl cyclase, a cyclic ADP-ribosyl (cADPr) hydrolase, and non-canonical receptor. The amino acid sequence of human CD38 is presented as SEQ ID NO: 14, and the cynomolgus monkey CD38 amino acid sequence is presented as SEQ ID NO: 15 (about 93% identical to human CD38). Human CD38 has a cytoplasmic domain from amino acids 1-21 of SEQ ID NO: 14, a transmembrane domain from amino acids 22-42 of SEQ ID NO: 14, and an extracellular domain from amino acids 43-300 of SEQ ID NO: 14.

### Anti-CD38 Antigen-Binding Domains

Provided herein is a binding molecule, e.g., an antibody or antibody-like molecule comprising an antigen-binding domain that specifically binds to CD38, e.g., a multimeric antibody comprising two, four, five, or six bivalent binding units, wherein each binding unit comprises two IgM or IgA heavy chain constant regions or multimerizing fragments or variants thereof, each associated with an antigen-binding domain. In certain embodiments, the antigen-binding domain comprises a heavy chain variable region (VH) and light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 182, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95.

In some embodiments, the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71. In some embodiments, the HCDR1, HCDR2, HCDR3, LCDR1, LCDR2, and LCDR3 comprise, respectively, the amino acid sequences of SEQ ID NO: 89, SEQ ID NO: 90, SEQ ID NO: 91, SEQ ID NO: 93, SEQ ID NO: 94, and SEQ ID NO: 95.

In some embodiments, the VH further comprises framework regions (HFWs) HFW1, HFW2, HFW3, and HFW4, and where the VL further comprises framework regions (LFWs) LFW1, LFW2, LFW3, and LFW4. In some embodiments, the framework regions are derived from a human antibody. In some embodiments, the framework regions are derived from a non-human antibody. The amino acids that make up the framework regions, respectively, can be readily identified for any given heavy or light chain variable region by one of ordinary skill in the art, since they have been structurally defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In some embodiments, the VH and VL comprise, respectively, an amino acid sequence at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142. In some embodiments, the VH and VL comprise, respectively, an amino acid sequence at least 80%, 85%, 90%, 95%, or 100% identical to the amino acid sequence of SEQ ID NO: 128 and SEQ ID NO: 133. In some embodiments, the VH and VL comprise, respectively, an amino acid sequence at least 80%, 85%, 90%, 95%, or 100% identical to the amino acid sequence of SEQ ID NO: 136 and SEQ ID NO: 138.

In certain embodiments, the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161.

In certain embodiments, the antigen-binding domain as provided above is an Fv fragment, e.g., a single-chain Fv fragment (scFv), or a disulfide-linked Fv fragment (sdFv). In certain embodiments, the antigen-binding domain as provided above is an scFv.

In certain embodiments, the antigen-binding domain as provided above is included in an antibody or antibody-like molecule as described elsewhere herein. In some embodiments, the antigen-binding domain as provided above is included in an antibody or antibody-like molecule, that is multispecific, e.g., bispecific, trispecific, or tetraspecific. In some embodiments, a multispecific antibody or antibody-like molecule as provided herein specifically binds to CD38 and to a target on an effector cell, e.g., CD16 or CD3.

In certain embodiments, the antibody or antibody-like molecule comprises a bivalent binding unit comprising two antigen-binding domains, where at least one antigen-binding domain specifically binds to CD38. According to this embodiment, the binding unit comprises two heavy chains each comprising a heavy chain constant region or fragment or variant thereof, and where at least one heavy chain constant region or fragment or variant thereof of the binding unit is associated with, e.g., fused to a copy of the provided VH of the antigen-binding domain. In certain embodiments, both heavy chain constant regions or fragments or variants thereof of the binding unit are associated with, e.g., fused to a copy of the provided VH of the antigen-binding domain. In certain embodiments, the heavy chains comprise IgG heavy chain constant regions or fragments or variants thereof. Various IgG heavy chain constant regions and fragments or variants thereof are known, such as those described in Kang, et al., 2019, Experimental & Molecular Medicine, 51:1-9; Brezski, et al., 2016, Current Opinion in Immunology, 40:62-69; Okazaki, et al., 2004, Journal of Molecular Biology, 336 (5): 1239-1249; Kang, et al., 2019, Front. Immunol., 10 (562): 1-11, and Saxena, et al., 2016, Front. Immunol., 7 (580): 1-11. In certain embodiments the single bivalent binding unit further comprises two light chains each comprising a light chain constant region or fragment or variant thereof. In certain embodiments at least one light chain constant region is associated with, e.g., fused to a copy of the provided VL of the antigen-binding domain. In certain embodiments both light chain constant regions or fragments or variants thereof of the binding unit are associated with, e.g., fused to a copy of the provided VL of the antigen-binding domain. In certain embodiments, the bivalent binding unit comprises a complete antibody, e.g., a complete IgG antibody. In certain embodiments, the single bivalent binding unit comprises a complete IgG antibody comprising two complete IgG heavy chain constant regions. In certain embodiments, the bivalent binding unit is a human IgG antibody, fragment, or derivative thereof.

In certain embodiments, the provided antigen-binding domain is included in a multimeric antibody or antibody-like molecule comprising two, four, five, or six bivalent binding units, where the antibody comprises four, eight, ten, or twelve antigen-binding domains. In certain embodiments at least one, two, three, four, five, six, seven, eight, nine, ten, eleven, or twelve of the antigen-binding domains specifically bind(s) to CD38. In certain embodiments, at least one, two, three, four, five, six, seven, eight, nine, ten, eleven, or twelve of the antigen-binding domains comprise the HCDR1, HCDR2, HCDR3, LCDR1, LCDR2, and LCDR3, and/or VH and VL amino acid sequences as provided above. According to these embodiments, each binding unit comprises two heavy chains each comprising an IgA or IgM constant region or a multimerizing fragment or variant thereof, and at least one of the heavy chain constant regions of the binding unit is associated with, e.g., fused to a copy of the provided VH of the provided antigen-binding domain. In certain embodiments the multimeric antibody or antibody-like molecule is a human antibody. In certain embodiments, multimeric antibody or antibody-like molecule is more potent than an equivalent amount of a bivalent IgG antibody comprising the VH and VL of the multimeric antibody. In some embodiments the multimeric can direct complement-directed cytotoxicity (CDC) of a CD38-expressing cell at a higher potency than an equivalent amount of a bivalent IgG antibody comprising the VH and VL of the multimeric antibody.

In certain embodiments, the provided multimeric antibody or antibody-like molecule is dimeric or tetrameric and comprises two or four bivalent IgA binding units and a J chain or functional fragment or variant thereof, where each binding unit comprises two IgA heavy chain constant regions, e.g., IgA1 or IgA2 heavy chain constant regions, or multimerizing fragments or variants thereof. In certain embodiments the dimeric or tetrameric antibody or antibody-like molecule can further comprise a secretory component, or fragment or variant thereof. In certain embodiments, the IgA heavy chain constant regions or multimerizing fragments or variants thereof each comprise a Cα3 domain and an α-tailpiece (αtp) domain, and can further comprise a Cα1 domain, a Cα2 domain, an IgA hinge region, or any combination thereof. In certain embodiments, multimeric antibody or antibody-like molecule is more potent than an equivalent amount of a bivalent IgG antibody comprising the VH and VL of the multimeric antibody.

In certain embodiments, the provided multimeric antibody or antibody-like molecule is hexameric or pentameric and comprises five or six bivalent IgM binding units, wherein each binding unit comprises two IgM heavy chain constant regions or multimerizing fragments or variants thereof. In certain embodiments the IgM heavy chain constant regions or multimerizing fragments or variants thereof each comprise a Cμ4 domain and a μ-tailpiece (μtp) domain, and can further comprise a Cμ1 domain, a Cμ2 domain, a Cμ3 domain, or any combination thereof. In certain embodiments the multimeric antibody antibody-like molecule is pentameric, and further comprises a J chain, or functional fragment thereof, or functional variant thereof. In certain embodiments, each binding unit further comprises two light chains each comprising a light chain constant region or fragment or variant thereof, and wherein at least one, two, three, four, five, six, seven eight, nine, ten, eleven, or twelve light chain constant regions are associated with, e.g., fused to a copy of the provided VL of the antigen-binding domain. In certain embodiments the multimeric antibody or antibody-like molecule is a human antibody. In certain embodiments, multimeric antibody or antibody-like molecule is more potent than an equivalent amount of a bivalent IgG antibody comprising the VH and VL of the multimeric antibody. In some embodiments the multimeric can direct complement-directed cytotoxicity (CDC) of a CD38-expressing cell at a higher potency than an equivalent amount of a bivalent IgG antibody comprising the VH and VL of the multimeric antibody.

The antibody or antibody-like molecule as provided herein can, in certain embodiments, be multispecific.

In certain embodiments the provided antigen-binding domain, or an antibody or fragment or derivative thereof, or antibody-like molecule comprising the antigen binding domain can specifically bind to human CD38, non-human primate CD38, or any combination thereof. In certain embodiments the non-human primate CD38 is cynomolgus monkey CD38. In certain embodiments the provided antigen-binding domain, or an antibody or fragment or derivative comprising the antigen binding domain binds to CD38 with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM, 0.005 nM, or 0.001 nM; and wherein the CD38 is human CD38, cynomolgus monkey CD38, or a combination thereof.

### IgM Antibodies, IgM-Like Antibodies, and IgM-Derived Binding Molecules

IgM is the first immunoglobulin produced by B cells in response to stimulation by antigen and is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal μ “tailpiece” (μtp). While several human alleles exist, the human IgM constant region typically comprises the amino acid sequence SEQ ID NO: 1 (IMGT allele IGHM*03, identical to, e.g., GenBank Accession No. pir∥S37768) or SEQ ID NO: 2 (IMGT allele IGHM*04, identical to, e.g., GenBank Accession No. sp|P01871.4). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cu 4 region ranges from about amino acid 329 to about amino acid 430 of SEQ ID NO: 1 or SEQ ID NO: 2, and the tailpiece ranges from about amino acid 431 to about amino acid 453 of SEQ ID NO: 1 or SEQ ID NO: 2.

Other forms of the human IgM constant region with minor sequence variations exist, including, without limitation, GenBank Accession Nos. CAB37838.1 and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Each IgM heavy chain constant region is typically associated with an antigen-binding domain, e.g., a scFv, or a subunit of an antigen-binding domain, e.g., a VH region.

Five IgM binding units can form a complex with an additional small polypeptide chain (the J-chain), or a functional fragment, variant, or derivative thereof, to form a pentameric IgM antibody or IgM-like antibody. The precursor form of the human J-chain is presented as SEQ ID NO: 6. The signal peptide extends from amino acid 1 to about amino acid 22 of SEQ ID NO: 6, and the mature human J-chain extends from about amino acid 23 to amino acid 159 of SEQ ID NO: 6. The mature human J-chain has the amino acid sequence SEQ ID NO: 7.

Exemplary variant and modified J-chains are provided elsewhere herein. Without the J-chain, an IgM antibody or IgM-like antibody typically assembles into a hexamer, comprising six binding units and up to twelve binding unit-associated antigen-binding domains. With a J-chain, an IgM antibody or IgM-like antibody typically assembles into a pentamer, comprising five binding units and up to ten binding unit-associated antigen-binding domains, or more, if the J-chain is a modified J-chain comprising one or more heterologous polypeptides that can be, e.g., additional J-chain-associated antigen-binding domain(s). The assembly of five or six IgM binding units into a pentameric or hexameric IgM antibody or IgM-like antibody is thought to involve interactions between the Cμ4 and μ tailpiece domains. See, e.g., Braathen, R., et al., J. Biol. Chem. 277:42755-42762 (2002). Accordingly, the constant regions of a pentameric or hexameric IgM antibody or antibody-like molecule provided in this disclosure typically includes at least the Cμ4 and/or μ tailpiece (μtp) domains. A “multimerizing fragment” of an IgM heavy chain constant region thus includes at least the Cμ4 domain and a μtp domain. An IgM heavy chain constant region can additionally include a Cμ3 domain or a fragment thereof, a Cμ2 domain or a fragment thereof, and/or a Cμ1 domain or a fragment thereof. In certain embodiments, a binding molecule, e.g., an IgM antibody or IgM-like antibody as provided herein can include a complete IgM heavy (μ) chain constant domain, e.g., SEQ ID NO: 1 or SEQ ID NO: 2, or a multimerizing variant, derivative, or analog thereof, e.g., as provided herein.

In certain embodiments, the disclosure provides a pentameric IgM or IgM-like antibody comprising five bivalent binding units, where each binding unit includes two IgM heavy chain constant regions or multimerizing fragments or variants thereof, each associated with an antigen-binding domain or a subunit of an antigen-binding domain. In certain embodiments, the two IgM heavy chain constant regions are human heavy chain constant regions.

Where the IgM or IgM-like antibody provided herein is pentameric, the IgM or IgM-like antibody typically further includes a J-chain, or functional fragment or variant thereof. In some embodiments, the J-chain is a modified J-chain comprising a heterologous moiety, e.g., a J-chain-associated antigen binding domain. In certain embodiments the J-chain-associated antigen binding domain specifically binds to an immune effector cell, e.g., a CD8+ cytotoxic T cell or an NK cell. In certain embodiments the modified J-chain includes one or more heterologous moieties attached thereto, e.g., an immune stimulatory agent. In certain embodiments the J-chain can be mutated to affect, e.g., enhance, the serum half-life of the IgM or IgM-like antibody provided herein, as discussed elsewhere in this disclosure. In certain embodiments the J-chain can be mutated to affect glycosylation, as discussed elsewhere in this disclosure.

In some embodiments, the IgM or IgM-like antibody provided herein is hexameric and comprises six bivalent binding units. In some embodiments, each binding unit comprises two IgM heavy chain constant regions or multimerizing fragments or variants thereof.

An IgM heavy chain constant region can include one or more of a Cμ1 domain or fragment or variant thereof, a Cμ2 domain or fragment or variant thereof, a Cμ3 domain or fragment or variant thereof, a Cμ4 domain or fragment or variant thereof, and/or a μ tail piece (μtp) or fragment or variant thereof, provided that the constant region can serve a desired function in the IgM or IgM-like antibody, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a μ tailpiece (μtp) or fragment or variant thereof, or a combination of a Cμ4 domain and a μtp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM or IgM-like antibody comprise two light chains. In some embodiments, the binding units of the IgM or IgM-like antibody comprise two fragments of light chains. In some embodiments, the light chains are kappa light chains. In some embodiments, the light chains are lambda light chains. In some embodiments, the light chains are hybrid kappa and lambda light chains. In some embodiments, each binding unit comprises two immunoglobulin light chains each comprising a VL situated amino terminal to an immunoglobulin light chain constant region.

IgM Antibodies, IgM-Like Antibodies, and IgM-Derived Binding Molecules with Enhanced Serum Half-Life

Certain IgM-derived multimeric binding molecules, e.g., antibodies or antibody-like molecules provided herein can be modified to have enhanced serum half-life. Exemplary IgM heavy chain constant region mutations that can enhance serum half-life of an IgM-derived binding molecule are disclosed in U.S. Pat. No. 10,899,835, which is incorporated by reference herein in its entirety. For example, a variant IgM heavy chain constant region of an IgM-derived binding molecule as provided herein can include an amino acid substitution at an amino acid position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

IgM Antibodies, IgM-Like Antibodies, and IgM-Derived Binding Molecules with Reduced CDC Activity

Certain IgM-derived multimeric binding molecules, e.g., antibodies or antibody-like molecules as provided herein can be engineered to exhibit reduced complement-dependent cytotoxicity (CDC) activity to cells in the presence of complement, relative to a reference IgM antibody or IgM-like antibody with a corresponding reference human IgM constant region identical, except for the mutations conferring reduced CDC activity. These CDC mutations can be combined with any of the mutations to block N-linked glycosylation and/or to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in U.S. Patent Publication No. 2021/0147567, which is incorporated herein by reference in its entirety. Assays for measuring CDC are well known to those of ordinary skill in the art, and exemplary assays are described e.g., in US Patent Application Publication No. 2021-0147567, which is incorporated by reference herein in its entirety.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and/or P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2 with aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position L310 of SEQ ID NO: 1 or SEQ ID NO: 2 with aspartic acid.

### Glyco-Modified IgM Antibodies, IgM-Like Antibodies, and IgM-Derived Binding Molecules

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine(S) or threonine (T). The glycan is attached to the nitrogen atom of the asparagine residue. See, e.g., Drickamer K, Taylor ME (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., PCT Application Publication No. WO 2021/041250, which is incorporated herein by reference in its entirety.

### IgA Antibodies, IgA-Like Antibodies, and IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019).

In some embodiments, the multimeric binding molecules are dimeric and comprise two bivalent binding units or variants or fragments thereof. In some embodiments, the multimeric binding molecules are dimeric or tetrameric, comprising two or four bivalent binding units or variants or fragments thereof, respectively, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In certain embodiments, the multimeric binding molecule provided by this disclosure is a dimeric binding molecule that includes four IgA heavy chain constant regions, or multimerizing fragments thereof, each associated with an antigen-binding domain for a total of four antigen-binding domains. As provided herein, a dimeric IgA antibody, IgA-derived binding molecule, or IgA-like antibody includes two binding units and a J-chain, e.g., a modified J-chain comprising a scFv antibody fragment that binds to CD3, or IL-15 and/or the IL-15 receptor-α sushi domain fused thereto as described elsewhere herein. Each binding unit as provided comprises two IgA heavy chain constant regions or multimerizing fragments or variants thereof. In certain embodiments, at least three or all four antigen-binding domains of the multimeric binding molecule bind to the same target antigen. In certain embodiments, at least three or all four binding polypeptides of the multimeric binding molecule are identical.

A bivalent IgA-derived binding unit includes two IgA heavy chain constant regions, and a dimeric IgA-derived binding molecule includes two binding units. IgA contains the following heavy chain constant domains, Cα1 (or alternatively CA1 or CH1), a hinge region, Cα2 (or alternatively CA2 or CH2), and Cα3 (or alternatively CA3 or CH3), and a C-terminal “tailpiece.” Human IgA has two subtypes, IgA1 and IgA2. The human IgA1 constant region typically includes the amino acid sequence SEQ ID NO: 3 The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα2 domain extends from about amino acid 125 to about amino acid 219 of SEQ ID NO: 3, the human Cα3 domain extends from about amino acid 228 to about amino acid 330 of SEQ ID NO: 3, and the tailpiece extends from about amino acid 331 to about amino acid 352 of SEQ ID NO: 3. The human IgA2 constant region typically includes the amino acid sequence SEQ ID NO: 4. The human Cα1 domain extends from about amino acid 6 to about amino acid 98 of SEQ ID NO: 4; the human IgA2 hinge region extends from about amino acid 102 to about amino acid 111 of SEQ ID NO: 4, the human Cα2 domain extends from about amino acid 113 to about amino acid 206 of SEQ ID NO: 4, the human Cα3 domain extends from about amino acid 215 to about amino acid 317 of SEQ ID NO: 4, and the tailpiece extends from about amino acid 318 to about amino acid 340 of SEQ ID NO: 4.

Two IgA binding units can form a complex with two additional polypeptide chains, the J-chain (e.g., SEQ ID NO: 7) and the secretory component (precursor, SEQ ID NO: 5, mature, from about amino acid 19 to about amino acid 764 of SEQ ID NO: 5) to form a bivalent secretory IgA (sIgA)-derived binding molecule as provided herein. The assembly of two IgA binding units into a dimeric IgA-derived binding molecule is thought to involve the Cα3 and tailpiece domains. See, e.g., Braathen, R., et al., J. Biol. Chem. 277:42755-42762 (2002). Accordingly, a multimerizing dimeric IgA-derived binding molecule provided in this disclosure typically includes IgA constant regions that include at least the Cα3 and a tailpiece domains. Four IgA binding units can likewise form a tetramer complex with a J-chain. A sIgA antibody can also form as a higher order multimer, e.g., a tetramer.

An IgA heavy chain constant region can additionally include a Cα2 domain or a fragment thereof, an IgA hinge region or fragment thereof, a Cα1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy (α) chain constant domain (e.g., SEQ ID NO: 3 or SEQ ID NO: 4), or a variant, derivative, or analog thereof. In some embodiments, the IgA heavy chain constant regions or multimerizing fragments thereof are human IgA constant regions.

In certain embodiments each binding unit of a multimeric binding molecule as provided herein includes two IgA heavy chain constant regions or multimerizing fragments or variants thereof, each including at least an IgA Cα3 domain and an IgA tailpiece domain. In certain embodiments the IgA heavy chain constant regions can each further include an IgA Cα2 domain situated N-terminal to the IgA Cα3 and IgA tailpiece domains. For example, the IgA heavy chain constant regions can include amino acids 125 to 353 of SEQ ID NO: 3 or amino acids 113 to 340 of SEQ ID NO: 4. In certain embodiments the IgA heavy chain constant regions can each further include an IgA or IgG hinge region situated N-terminal to the IgA Cα2 domains. For example, the IgA heavy chain constant regions can include amino acids 102 to 353 of SEQ ID NO: 3 or amino acids 102 to 340 of SEQ ID NO: 4. In certain embodiments the IgA heavy chain constant regions can each further include an IgA Cα1 domain situated N-terminal to the IgA hinge region.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments light chains. In some embodiments, the light chains are kappa light chains. In some embodiments, the light chains are lambda light chains. In some embodiments the light chains are chimeric kappa-lambda light chains. In some embodiments, each binding unit comprises two immunoglobulin light chains each comprising a VL situated amino terminal to an immunoglobulin light chain constant region.

Modified and/or Variant J-Chains

In certain embodiments, the multimeric binding molecule, e.g., antibody or antibody-like molecule provided herein comprises a J-chain or functional fragment or variant thereof. In certain embodiments, the multimeric binding molecule provided herein is a pentameric IgM antibody or IgM antibody-like molecule and comprises a J-chain or functional fragment or variant thereof. In certain embodiments, the multimeric binding molecule provided herein is a dimeric IgA antibody or IgA antibody-like molecule and comprises a J-chain or functional fragment or variant thereof. In some embodiments, the multimeric binding molecule can comprise a naturally occurring J-chain, such as a mature human J-chain (e.g., SEQ ID NO: 7). In some embodiments, the multimeric binding molecule can comprise a functional fragment or functional variant of a naturally occurring J-chain.

In certain embodiments, the J-chain of a pentameric an IgM or IgM-like antibody or a dimeric IgA or IgA-like antibody as provided herein can be modified, e.g., by introduction of a heterologous moiety, or two or more heterologous moieties, e.g., polypeptides, without interfering with the ability of the IgM or IgM-like antibody or IgA or IgA-like antibody to assemble and bind to its binding target(s). See U.S. Pat. Nos. 9,951,134, 10,975,147, 10,400,038, and 10,618,978, and U.S. Patent Application Publication No. US-2019-0185570, each of which is incorporated herein by reference in its entirety. Accordingly, IgM or IgM-like antibodies or IgA or IgA-like antibodies as provided herein, including bispecific or multispecific IgM or IgM-like antibodies or IgA or IgA-like antibodies as described elsewhere herein, can include a modified J-chain or functional fragment or variant thereof that further includes a heterologous moiety, e.g., a heterologous polypeptide, introduced into the J-chain or fragment or variant thereof. In certain embodiments heterologous moiety can be a peptide or polypeptide fused in frame or chemically conjugated to the J-chain or fragment or variant thereof. For example, the heterologous polypeptide can be fused to the J-chain or functional fragment or variant thereof. In certain embodiments, the heterologous polypeptide is fused to the J-chain or functional fragment or variant thereof via a linker, e.g., a peptide linker consisting of least 5 amino acids, but typically no more than 25 amino acids. In certain embodiments, the peptide linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), or GGGGSGGGGSGGGGSGGGGSGGGGS (SEQ ID NO: 13). In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a stabilizing peptide that can increase the half-life of the IgM or IgM-like antibody, or a chemical moiety such as a polymer or a cytotoxin. In some embodiments, the heterologous moiety comprises a stabilizing peptide that can increase the half-life of the binding molecule, e.g., human serum albumin (HSA) or an HSA binding molecule.

In some embodiments, a modified J-chain includes a J-chain-associated antigen-binding domain, e.g., a polypeptide capable of specifically binding to a target antigen. In certain embodiments, a J-chain-associated antigen-binding domain can be an antibody, or an antigen-binding fragment thereof, as described elsewhere herein. In certain embodiments the J-chain-associated antigen-binding domain can be a single chain Fv (scFv) antigen-binding domain or a single-chain antigen-binding domain derived, e.g., from a camelid or condricthoid antibody. The J-chain-associated antigen-binding domain can be introduced into the J-chain at any location that allows the binding of the J-chain-associated antigen-binding domain to its binding target without interfering with J-chain function or the function of an associated IgM or IgA antibody or antibody-like molecule. Insertion locations include but are not limited to at or near the C-terminus, at or near the N-terminus or at an internal location that, based on the three-dimensional structure of the J-chain, is accessible. In certain embodiments, the J-chain-associated antigen-binding domain can be introduced into the mature human J-chain of SEQ ID NO: 7 between cysteine residues 92 and 101 of SEQ ID NO: 7. In a further embodiment, the J-chain-associated antigen-binding domain can be introduced into the human J-chain of SEQ ID NO: 7 at or near a glycosylation site. In a further embodiment, the J-chain-associated antigen-binding domain can be introduced into the human J-chain of SEQ ID NO: 7 within about 10 amino acid residues from the C-terminus, or within about 10 amino acids from the N-terminus. As described elsewhere herein, this disclosure provides a multimeric, bispecific binding molecule comprising a modified J-chain, where the modified J-chain comprises a J-chain-associated antigen binding domain that specifically binds to an immune effector cell, e.g., a T cell such as a CD4+ T cell or a CD8+ cytotoxic T cell or an NK cell.

In some embodiments, a modified J-chain can further include an immune stimulatory agent (ISA), e.g., cytokine, e.g., interleukin-2 (IL-2) or interleukin-15 (IL-15), or a receptor-binding fragment or variant thereof, which in certain embodiments can be associated, either via binding or covalent attachment, with part of its receptor, e.g., the sushi domain of IL-15 receptor-α. Such ISAs are described in detail in PCT Publication No. WO 2021/030688, which is incorporated herein by reference in its entirety.

In certain embodiments, the J-chain of an IgM antibody, IgM-like antibody, IgA antibody, IgA-like antibody, or IgM- or IgA-derived binding molecule as provided herein is a variant J-chain that comprises one or more amino acid substitutions that can alter, e.g., the serum half-life of an IgM antibody, IgM-like antibody, IgA antibody, IgA-like antibody, or IgM- or IgA-derived binding molecule provided herein. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

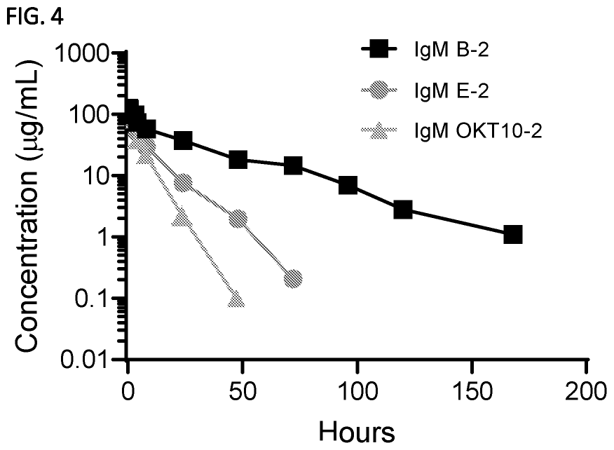

In some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., U.S. Pat. No. 10,899,835, which is incorporated herein by reference in its entirety. In certain embodiments, the variant J-chain can comprise an amino acid substitution at the amino acid position corresponding to amino acid Y102 of the mature wild-type human J-chain (SEQ ID NO: 7). By “an amino acid corresponding to amino acid Y102 of the mature wild-type human J-chain” is meant the amino acid in the sequence of the J-chain of any species which is homologous to Y102 in the human J-chain. See U.S. Pat. No. 10,899,835. The position corresponding to Y102 in SEQ ID NO: 7 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 7 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the mutant J-chain. IgM antibodies, IgM-like antibodies, and IgM-derived binding molecules comprising a mutation at the amino acid corresponding to Y102 of SEQ ID NO: 7 have an improved serum half-life when administered to an animal than a corresponding antibody, antibody-like molecule or binding molecule that is identical except for the substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine(S) or arginine (R). In a particular embodiment, the amino acid corresponding to Y102 of SEQ ID NO: 7 can be substituted with alanine. In a particular embodiment the J-chain or functional fragment or variant thereof is a variant human J-chain and comprises the amino acid sequence SEQ ID NO: 8, a J chain referred to herein as “J*”.

Wild-type J-chains typically include one N-linked glycosylation site. In certain embodiments, a variant J-chain or functional fragment thereof of a multimeric binding molecule as provided herein includes a mutation within the asparagine (N)-linked glycosylation motif N-X₁-S/T, e.g., starting at the amino acid position corresponding to amino acid 49 (motif N6) of the mature human J-chain (SEQ ID NO: 7) or J* (SEQ ID NO: 8), where N is asparagine, X₁ is any amino acid except proline, and S/T is serine or threonine, and where the mutation prevents glycosylation at that motif. As demonstrated in U.S. Pat. No. 10,899,835, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a binding molecule comprising a J-chain as provided herein can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J-chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine(S) or aspartic acid (D). In a particular embodiment, the position corresponding to N49 of SEQ ID NO: 7 or SEQ ID NO: 8 can be substituted with alanine (A). In another embodiment, the position corresponding to N49 of SEQ ID NO: 7 or SEQ ID NO: 8 can be substituted with aspartic acid (D). In some embodiments, the position corresponding to S51 of SEQ ID NO: 7 or SEQ ID NO: 8 is substituted with alanine (A) or glycine (G). In some embodiments, the position corresponding to S51 of SEQ ID NO: 7 or SEQ ID NO: 8 is substituted with alanine (A).

Multimeric Bispecific or Multispecific Anti-CD38 Binding Molecules with a Modified J-Chain that Binds to an Immune Effector Cell.

This disclosure provides a multimeric, bispecific or multispecific binding molecule for use in treating cancers, e.g., hematologic cancers, e.g., Multiple Myeloma (MM). In certain embodiments, the binding molecule is bispecific and targets CD38 on cancer cells with high avidity, while also targeting an immune effector cell, e.g., a CD4+ or CD8+ T cell or an NK cell via a single antigen-binding domain, thereby facilitating effector cell-mediated killing of the cancer cells while at the same time minimizing excessive release of cytokines. In certain embodiments the multimeric, bispecific, anti-CD38 binding molecule is an anti-CD38× anti-CD3 binding molecule.

Accordingly, the disclosure provides a multimeric, bispecific or multispecific binding molecule comprising two IgA or IgA-like or five IgM or IgM-like bivalent binding units and a modified J-chain, where the modified J-chain includes at least a wild-type J-chain or a functional fragment or variant thereof and a J-chain-associated antigen-binding domain that specifically binds to an immune effector cell. Each binding unit comprises two antibody heavy chains, each comprising an IgA, IgA-like, IgM, or IgM-like heavy chain constant region or multimerizing fragment thereof (as described elsewhere herein) and at least a heavy chain variable region (VH) portion of a binding unit-associated antigen-binding domain. At least three, at least four, at least five, at least six, at least seven, at least eight, at least nine, or all ten of the binding unit-associated antigen-binding domains specifically bind to CD38. A binding molecule as provided herein can induce immune effector cell-dependent killing of cells, e.g., cancer cells, expressing CD38.

In certain embodiments, the modified J-chain of the binding molecule provided herein includes a variant of a wild-type J-chain or fragment thereof, where the variant includes one or more single amino acid substitutions, deletions, or insertions relative to a wild-type J-chain that can affect serum half-life of the binding molecule; and wherein the binding molecule exhibits an increased serum half-life upon administration to an animal relative to a reference binding molecule that is identical except for the one or more single amino acid substitutions, deletions, or insertions in the J-chain, and is administered in the same way to the same animal species. For example, in certain embodiments the J-chain is a variant human J-chain that comprises the amino acid sequence SEQ ID NO: 8 (“J*”).

In certain embodiments, the J-chain-associated antigen-binding domain of the provided binding molecule comprises an antibody or fragment thereof. In certain embodiments the antibody fragment is a single chain Fv (scFv). The scFv can be fused or chemically conjugated to the J-chain or fragment or variant, e.g., J*. In certain embodiments, the scFv is fused to the J-chain via a peptide linker e.g., SEQ ID NOs: 9-13. As noted elsewhere in the disclosure, the scFv can be fused to J-chain or fragment or variant thereof in any way so long as the function of the J-chain, i.e., to assemble with IgM, IgM-like, IgA, or IgA-like binding units to form a dimer or a pentamer, is not affected. For example, the scFv can be fused to the N-terminus of the J-chain or fragment or variant thereof, the C-terminus of the J-chain or fragment or variant thereof, or to both the N-terminus and C-terminus of the J-chain or fragment or variant thereof.

The immune effector cell bound by the antigen binding domain of the modified J-chain can be any immune effector cell confers a beneficial effect when associated with a cancer cell targeted by CD38, for example mediating cell-based killing of the CD38+ cancer cell. In certain embodiments the immune effector cell can be, without limitation, a T cell, e.g., a CD4+ T cell, a CD8+ T cell, an NKT cell, or a γδ T cell, a B cell, a plasma cell, a macrophage, a dendritic cell, or a natural killer (NK) cell. In certain embodiments the immune effector cell is a T cell, e.g., a CD4+ or CD8+ T cell. In certain embodiments the immune effector cell is a CD8+ cytotoxic T cell. In certain embodiments the immune effector cell is an NK cell.

Where the immune effector cell is a T cell, for example a CD8+ T cell, the J-chain-associated antibody or fragment thereof, e.g., scFv, can specifically bind to the T cell surface antigen CD3, e.g., CD38. In certain embodiments the anti-CD3 scFv comprises a scFv heavy chain variable region (scFv VH) and a scFv light chain variable region (scFv VL), wherein the scFv VH comprises scFv VH complementarity-determining regions VHCDR1, VHCDR2, and VHCDR3 and the scFv VL comprises scFv VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49. In some embodiments, the VHCDR1, VHCDR2, VHCDR3, VLCDR1, VLCDR2, and VLCDR3 comprise, respectively, SEQ ID NO: 25, SEQ ID NO: 26, SEQ ID NO: 27, SEQ ID NO: 29, SEQ ID NO: 30, and SEQ ID NO: 31. In some embodiments, the scFv VH and scFv VL comprise an amino acid sequence at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively. In some embodiments, the scFv fragment comprises the scFv VH and scFv VL amino acid sequences SEQ ID NO: 24 and SEQ ID NO: 28, respectively.

In certain other embodiments, the immune effector cell is an NK cell, and the J-chain-associated antibody or fragment thereof, e.g., scFv, can specifically bind to CD16 or CD56. Many CD16 and CD56 scFv are known, such as those disclosed in U.S. Pat. Nos. 9,035,026, 9,701,750, 10,730,941, 11,001,633, McCall et al., 1999. Mol Immunol. 7:433-445.

A modified J-chain of a multimeric, bispecific, anti-CD38 binding molecule, e.g., an anti-CD38× anti-CD3 binding molecule as provided herein can be further modified to include additional heterologous moieties attached to the J-chain. Exemplary moieties are described, e.g., in U.S. Pat. No. 9,951,134, and in U.S. Patent Application Publication Nos. US 2019-0185570 and U.S. Pat. No. 10,618,978, and in PCT Publication No. WO2021/030688, all of which are incorporated herein by reference in their entireties. In certain embodiments, the modified J-chain of a multimeric, bispecific anti-CD38 binding molecule, e.g., an anti-CD38× anti-CD3 binding molecule as provided herein can further include an immune stimulatory agent (“ISA”) fused or chemically conjugated to the J-chain or fragment or variant thereof. For example, the ISA can include a cytokine or receptor-binding fragment or variant thereof. In a particular embodiment, a J-chain-associated ISA can include (a) an interleukin-15 (IL-15) protein or receptor-binding fragment or variant thereof (“I”), and (b) an interleukin-15 receptor-α (IL-15Rα) fragment comprising the sushi domain or a variant thereof capable of associating with I (“R”), wherein the J-chain or fragment or variant thereof and at least one of I and R, or both I and R, are associated as a fusion protein, and wherein I and R can associate to function as the ISA. In certain embodiments, the ISA can be fused to the J-chain via a peptide linker.

### Polynucleotides, Vectors, and Host Cells

The disclosure further provides a polynucleotide, e.g., an isolated, recombinant, and/or non-naturally occurring polynucleotide, that includes a nucleic acid sequence that encodes an antigen-binding domain as provided herein or a polypeptide subunit of an antibody or antibody-like molecule, e.g., a dimeric, hexameric, or pentameric antibody or antibody-like molecule as provided herein. By “polypeptide subunit” is meant a portion of an antibody or antibody-like molecule, binding unit, or antigen-binding domain that can be independently translated. Examples include, without limitation, an antibody variable domain, e.g., a VH or a VL, a J chain, including modified J-chains as provided herein, a secretory component, a single chain Fv, an antibody heavy chain, an antibody light chain, an antibody heavy chain constant region, an antibody light chain constant region, and/or any fragment, variant, or derivative thereof.

In certain embodiments, the polynucleotide comprising a nucleic acid sequence that encodes a polypeptide subunit of a binding molecule described herein. In some embodiments, the polynucleotide encodes a polypeptide subunit comprising a heavy chain constant region and at least an antibody VH portion of the binding domain of the binding molecule. In some embodiments, the polynucleotide encodes a polypeptide subunit comprising the heavy chain of the binding molecule. In some embodiments, the polynucleotide encodes a polypeptide subunit comprises a VH comprising HCDR1, HCDR2, and HCDR3 regions, wherein the HCDR1, HCDR2, and HCDR3 comprise, respectively, the amino acid sequences of SEQ ID NO: 65, SEQ ID NO: 66, and SEQ ID NO: 67; SEQ ID NO: 57, SEQ ID NO: 58, and SEQ ID NO: 59; SEQ ID NO: 73, SEQ ID NO: 74, and SEQ ID NO: 75; SEQ ID NO: 81, SEQ ID NO: 82, and SEQ ID NO: 83; SEQ ID NO: 89, SEQ ID NO: 90, and SEQ ID NO: 91; SEQ ID NO: 97, SEQ ID NO: 98, and SEQ ID NO: 99; SEQ ID NO: 105, SEQ ID NO: 106, and SEQ ID NO: 107; SEQ ID NO: 113, SEQ ID NO: 114, and SEQ ID NO: 115; SEQ ID NO: 121, SEQ ID NO: 122, and SEQ ID NO: 123; SEQ ID NO: 65, SEQ ID NO: 134, and SEQ ID NO: 67; SEQ ID NO: 65, SEQ ID NO: 182, and SEQ ID NO: 67; or SEQ ID NO: 65, SEQ ID NO: 135, and SEQ ID NO: 67.

In some embodiments, the polynucleotide encodes a polypeptide subunit comprising a light constant region and at least an antibody VL portion of the binding domain of the binding molecule. In some embodiments, the polynucleotide encodes a polypeptide subunit comprising the light chain of the binding molecule. In some embodiments, the polynucleotide encodes a polypeptide subunit comprises a VL comprising LCDR1, LCDR2, and LCDR3 regions, wherein the LCDR1, LCDR2, and LCDR3 comprise, respectively, the amino acid sequences of SEQ ID NO: 69, SEQ ID NO: 70, and SEQ ID NO: 71; SEQ ID NO: 61, SEQ ID NO: 62, and SEQ ID NO: 63; SEQ ID NO: 77, SEQ ID NO: 78, and SEQ ID NO: 79; SEQ ID NO: 85, SEQ ID NO: 86, and SEQ ID NO: 87; SEQ ID NO: 93, SEQ ID NO: 94, and SEQ ID NO: 95; SEQ ID NO: 101, SEQ ID NO: 102, and SEQ ID NO: 103; SEQ ID NO: 109, SEQ ID NO: 110, and SEQ ID NO: 111; SEQ ID NO: 117, SEQ ID NO: 118, and SEQ ID NO: 119; SEQ ID NO: 125, SEQ ID NO: 126, and SEQ ID NO: 127; SEQ ID NO: 143, SEQ ID NO: 94, and SEQ ID NO: 95; SEQ ID NO: 144, SEQ ID NO: 94, and SEQ ID NO: 95; SEQ ID NO: 145, SEQ ID NO: 94, and SEQ ID NO: 95; or SEQ ID NO: 146, SEQ ID NO: 94, and SEQ ID NO: 95.

In certain embodiments, the polypeptide subunit can include an IgM heavy chain constant region or IgM-like heavy chain constant region or multimerizing fragment thereof, or an IgA heavy chain constant region or IgA-like heavy chain constant region or multimerizing fragment thereof, which is associated with, e.g., fused to an antigen-binding domain or a subunit thereof, e.g., to the VH portion of an antigen-binding domain or the VL portion of an antigen binding domain, all as provided herein. In certain embodiments the polynucleotide can encode a polypeptide subunit that includes a human IgM heavy chain constant region, a human IgM-like heavy chain constant region, a human IgA heavy chain constant region, a human IgA-like heavy chain constant region, or multimerizing fragment thereof, e.g., SEQ ID NO: 1, SEQ ID NO: 2, SEQ ID NO: 3, or SEQ ID NO: 4, any of which is associated with, e.g., fused to an antigen-binding domain or subunit thereof, e.g., the C-terminal end of a VH.

To form the antigen-binding domains or the variable regions of antibodies that specifically bind to CD38, the provided polynucleotides can be inserted into expression vector templates, e.g., for a monomeric antibody, e.g., an IgG antibody, or for IgM and/or IgA structures, thereby creating monomeric antibodies comprising a single binding unit, or multimeric antibodies or multimerizing antibody-like molecule having at least two bivalent binding units. In brief, nucleic acid sequences encoding the heavy and light chain variable domain sequences can be synthesized or amplified from existing molecules and inserted into vectors in the proper orientation and in frame such that upon expression, the vector will yield a full length heavy or light chain. Vectors useful for these purposes are known in the art. Such vectors can also comprise enhancer and other sequences needed to achieve expression of the desired chains. Multiple vectors or single vectors can be used. These vectors are transfected into host cells and then the chains are expressed and purified. Upon expression the chains form fully functional multimeric binding molecules, as has been reported in the literature. The fully assembled multimeric binding molecules can then be purified by standard methods. The expression and purification processes can be performed at commercial scale, if needed.

The disclosure further provides a composition comprising two or more polynucleotides, where the two or more polynucleotides collectively can encode an antigen-binding domain or an antibody or antibody-like molecule, e.g., a monomeric, dimeric, hexameric, or pentameric antibody as described herein. In certain embodiments the composition can include a polynucleotide encoding an IgG, IgM, and/or IgA heavy chain or fragment thereof, e.g., a human IgG, IgM, or IgA heavy chain as described above, where the IgG, IgM, and/or IgA heavy chain comprises at least the provided VH of a CD38 antigen-binding domain as provided herein, and a polynucleotide encoding a light chain or fragment thereof, e.g., a human kappa or lambda light chain that comprises at least the provided VL of a CD38 antigen-binding domain as provided herein. A polynucleotide composition as provided can further include a polynucleotide encoding a J chain, e.g., a human J chain, or a fragment, variant, or derivative thereof. In certain embodiments the polynucleotides making up a composition as provided herein can be situated on two, three, or more separate vectors, e.g., expression vectors. Such vectors are provided by the disclosure. In certain embodiments, two or more of the polynucleotides making up a composition as provided herein can be situated on a single vector, e.g., an expression vector. Such a vector is provided by the disclosure.

In certain embodiments, this disclosure provides a composition comprising two, three, or more polynucleotides as provided herein, where the polynucleotides together can encode an anti-CD38 binding molecule, e.g., a multimeric, bispecific anti-CD38 binding molecule, e.g., an anti-CD38× anti-CD3 binding molecule as provided herein. In certain embodiments the polynucleotides can be situated on separate vectors. In certain embodiments two or more of the polynucleotides can be situated on the same vector. Such vectors are likewise provided by the disclosure.

The disclosure further provides a host cell, e.g., a prokaryotic or eukaryotic host cell, that includes a polynucleotide or two or more polynucleotides encoding an anti-CD38 binding molecule, e.g., a multimeric, bispecific, anti-CD38 binding molecule, e.g., an anti-CD38× anti-CD3 binding molecule as provided herein, or any subunit thereof, a polynucleotide composition as provided herein, or a vector or two, three, or more vectors that collectively encode the binding molecule as provided herein, or any subunit thereof.

In a related embodiment, the disclosure provides a method of producing a multimeric binding molecule as provided by this disclosure, where the method comprises culturing a host cell as provided herein and recovering the multimeric binding molecule.

### Methods of Use

The disclosure further provides a method of treating a disease or disorder, e.g., cancer or other malignancy, e.g., a hematologic cancer or malignancy, in a subject in need of treatment, comprising administering to the subject a therapeutically effective amount of an anti-CD38 antibody or antigen-binding fragment or derivative thereof, e.g., an anti-CD38× anti-CD3 antibody, as provided herein. By “therapeutically effective dose or amount” or “effective amount” is intended an amount of the binding molecule that when administered brings about a positive response, e.g., killing of tumor cells, in the subject.

In certain embodiments the cancer to be treated can be any cancer in which the malignant cells express or over-express CD38. For example, the cancer can be a hematological cancer, such as chronic lymphocytic leukemia (CLL), multiple myeloma (MM), Hodgkin's lymphoma (HL), diffuse large B-cell lymphoma (DLBCL), peripheral T-cell lymphoma (PTCL), and various solid tumors, including prostate cancer, non-small cell lung cancer (NSCLC), squamous cell carcinoma of the head and neck (SCCHN), ovarian cancer, and liver cancer. In some embodiments, the cancer is a hematological cancer. In some embodiments, the cancer is MM.

Effective doses of compositions for treatment of cancer vary depending upon many different factors, including means of administration, target site, physiological state of the subject, whether the subject is human or an animal, and other medications administered. Usually, the subject is a human, but non-human mammals including transgenic mammals can also be treated.

The subject to be treated can be any mammal in need of treatment, in certain embodiments, the subject is a human subject.

In its simplest form, a preparation to be administered to a subject is an anti-CD38 antibody or antigen-binding fragment or derivative thereof, e.g., an anti-CD38× anti-CD3 antibody as provided herein, administered in conventional dosage form, which can be combined with a pharmaceutical excipient, carrier or diluent as described elsewhere herein.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

### Pharmaceutical Compositions and Administration Methods

The components of compositions comprising an anti-CD38 antibody or antigen-binding fragment or derivative thereof, e.g., an anti-CD38× anti-CD3 antibody as provided herein, may differ depending on the route of administration. Exemplary routes of administration include intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, for intratumoral, intravenous, or intraarterial injection or drip. A suitable pharmaceutical composition can comprise a buffer (e.g., acetate, phosphate, or citrate buffer), a surfactant (e.g., polysorbate), optionally a stabilizer agent (e.g., human albumin), etc.

As discussed herein, an anti-CD38 antibody or antibody-like molecule, e.g., a multimeric anti-CD38× anti-CD3 antibody as provided herein can be administered in a pharmaceutically effective amount for the treatment of a subject in need thereof. In this regard, it will be appreciated that the disclosed antibodies or antigen-binding fragments or derivatives thereof can be formulated to facilitate administration and promote stability of the active agent. Pharmaceutical compositions accordingly can comprise a pharmaceutically acceptable, non-toxic, sterile carrier such as physiological saline, non-toxic buffers, preservatives, and the like. A pharmaceutically effective amount of an antibody or antigen-binding fragment or derivative thereof as provided herein means an amount sufficient to achieve effective binding to a target and to achieve a therapeutic benefit. Suitable formulations are described in Remington's Pharmaceutical Sciences, e.g., 21^{st} Edition (Lippincott Williams & Wilkins) (2005).

Certain pharmaceutical compositions provided herein can be orally administered in an acceptable dosage form including, e.g., capsules, tablets, aqueous suspensions, or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of an anti-CD38 antibody or antibody-like molecule, e.g., a multimeric anti-CD38× anti-CD3 antibody disclosed herein that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic response).

In keeping with the scope of the present disclosure, an anti-CD38 antibody or antibody-like molecule, e.g., a multimeric anti-CD38× anti-CD3 antibody as provided herein can be administered to a subject in need of therapy in an amount sufficient to produce a therapeutic effect. An anti-CD38 antibody or antibody-like molecule, e.g., an anti-CD38× anti-CD3 antibody, as provided herein can be administered to the subject in a conventional dosage form prepared by combining the antibody or antibody-like molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of an anti-CD38 antibody or antibody-like molecule, e.g., a multimeric anti-CD38× anti-CD3 antibody as provided herein in the manufacture of a medicament for treating cancer or other malignancy. The disclosure also provides for an anti-CD38 antibody or antibody-like molecule, e.g., an anti-CD38× anti-CD3 antibody, as provided herein for use in treating cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Generation of CD38 Binders

An antibody discovery campaign to generate CD38 binding antibodies was done using a commercial vendor (Antibody Solutions). Three mouse strains Balb/C, NZB/W, and CD1 were immunized with human and cynomolgus monkey (cyno) CD38 extracellular domain proteins from ACROBiosystems. Two different adjuvants were used for the immunizations: alhydrogel/muramyl dipeptide (ALD/MDP) and Sigma Adjuvant System®/RIBI (SAS/RIBI). A total of six hybridoma libraries were generated. Hybridoma supernatants were screened to identify binding of anti-CD38 antibodies to recombinant human CD38 and recombinant cyno CD38 using an enzyme-linked immunosorbent assay (ELISA). In addition, supernatants were screened to identify anti-CD38 antibodies that bind cells expressing human CD38. 48 clones that bind both human and cyno CD38 were expanded and sequenced. Following sequencing, nine clones comprising unique sets of CDR sequences were identified. Sequences of the anti-CD38 murine hybridoma clones are shown in Table 2.

TABLE 2
Sequences of Hybridoma Clones
Clone A

| SEQ ID | VH | | | | |
|---|---|---|---|---|---|
| 56 | QVQLQQSGAELVKPGASVKISCKASGYTFSNYWIEWVKQRPGLGLEWIGDILPGSGTTNYNEKEKGKAT | | | | |
| | FTADTSSNTAYMQLSSLTSEDSAVYYCAGYGNYLWFTYWGQGTLVTVSA | | | | |
| SEQ ID | HCDR1 Sequence | SEQ ID | HCDR2 Sequence | SEQ ID | HCDR3 Sequence |
| 57 | GYTFSNYWIE | 58 | DILPGSGTTNYNEKEKG | 59 | YGNYLWFTY |
| SEQ ID | VL | | | | |
| 60 | DIQMNQSPSSLSASLGDTITITCHASQNINVWLSWYQQKPGNIPKLLIYKASNLHTGVPSRFSGSGSGT | | | | |
| | GFTLTISSLQPEDIATYYCQQGQSYPYTFGGGTKLEIK | | | | |
| SEQ ID | LCDR1 Sequence | SEQ ID | LCDR2 Sequence | SEQ ID | LCDR3 Sequence |
| 61 | HASQNINVWLS | 62 | KASNLHT | 63 | QQGQSYPYT |

Clone B

| SEQ ID | VH | | | | |
|---|---|---|---|---|---|
| 64 | QAYLQQSGAEVVRSGASVKMSCKAAGYTFTSYNMHWVKQTPGQGLEWIGYIYPGNGGTNYNQKEKGKAT | | | | |
| | LTADTSSTTAYMQISSLTSEDSAVYFCARGASMITTGAWFAYWGQGTLVTVSA | | | | |
| SEQ ID | HCDR1 Sequence | SEQ ID | HCDR2 Sequence | SEQ ID | HCDR3 Sequence |
| 65 | GYTFTSYNMH | 66 | YIYPGNGGTNYNQKFKG | 67 | GASMITTGAWFAY |
| SEQ ID | VL | | | | |
| 68 | NIVLTQSPASLAVSLGQRATISCRASESLDTYGNSEMYWYQQKPGQPPKLLIYLASSLESGVPARESGS | | | | |
| | GSRTDFTLTIDPVEADDAATYYCHQNHEDPWTFGGGTKLEIK | | | | |
| SEQ ID | LCDR1 Sequence | SEQ ID | LCDR2 Sequence | SEQ ID | LCDR3 Sequence |
| 69 | RASESLDTYGNSFMY | 70 | LASSLES | 71 | HQNHEDPWT |

Clone C

| SEQ ID | VH | | | | |
|---|---|---|---|---|---|
| 72 | EVQLQQSGTVLARPGASVKMSCKASGYTFTRYWMHWVKQRPGQDLEWIGGFYPGNSDTRYNQKEKDKAK | | | | |
| | LTAVTSTSTAYMELSSLTNEDSAVYYCTRGDRHDEGFAYWGQGTLVTVSA | | | | |
| SEQ ID | HCDR1 Sequence | SEQ ID | HCDR2 Sequence | SEQ ID | HCDR3 Sequence |
| 73 | GYTFTRYWMH | 74 | GFYPGNSDTRYNQKFKD | 75 | GDRHDEGFAY |
| SEQ ID | VL | | | | |
| 76 | DIQMTQSPASLSASVGETVTITCRASGNIHNYLAWYQQKQGKSPQLLVYNAKTLPDGVPSRESGSGSGT | | | | |
| | QYSLKINSLQPEDFGTYYCQNIWINYTFGGGTKLEIK | | | | |
| SEQ ID | LCDR1 Sequence | SEQ ID | LCDR2 Sequence | SEQ ID | LCDR3 Sequence |
| 77 | RASGNIHNYLA | 78 | NAKTLPD | 79 | QNIWTNYT |

Clone D

| SEQ ID | VH | | | | |
|---|---|---|---|---|---|
| 80 | QIQLVQSGPELKKPGETVKISCKASGYTFTNYGMNWVKQAPGEDLKWMGWLNTNTGEPTYAEEFKGRFA | | | | |
| | FSLETSASTAYLQINNLRNEDTATYFCARWYYGSSDNSYAMDYWGQGTSVTVSS | | | | |
| SEQ ID | HCDR1 Sequence | SEQ ID | HCDR2 Sequence | SEQ ID | HCDR3 Sequence |
| 81 | GYTFTNYGMIN | 82 | WLNTNTGEPTYAEEFKG | 83 | WYYGSSDNSYAMDY |
| SEQ ID | VL | | | | |
| 84 | ETIVTQFPASLSMAIGEKVTIRCITSTDIDDDMNWYQQRPGEPPKLLISEGNTLRPGVPSRESSSGYGT | | | | |
| | DFVFTFENMLSEDVADYYCLQSDSLPYTVGGGTRLEIK | | | | |
| SEQ ID | LCDR1 Sequence | SEQ ID | LCDR2 Sequence | SEQ ID | LCDR3 Sequence |
| 85 | ITSTDIDDDMN | 86 | EGNTLRP | 87 | LOSDSLPYT |

Clone E

| SEQ ID | VH | | | | |
|---|---|---|---|---|---|
| 88 | QVQLQQSGAELVRPGTSVKMSCKAAGYTFTNHWIEWVKQRPGHGLEWIGDIFPGGGYTDYNEKFKGKAT | | | | |
| | LTADTSSSTAYMQLSSLTSEDSAIYYCARGRDLAMDYWGQGTSVTVSS | | | | |
| SEQ ID | HCDR1 Sequence | SEQ ID | HCDR2 Sequence | SEQ ID | HCDR3 Sequence |
| 89 | GYTFTNHWIE | 90 | DIFPGGGYTDYNEKFKG | 91 | GRDLAMDY |
| SEQ ID | VL | | | | |
| 92 | DIQMNQSPSSLSASLGDTITITCHASQNINVWLGWYQQKPGNIPKLLIYKASNLHTGVPSRFSGSGSGT | | | | |
| | GFTLTISSLQPEDIATYYCQQGQSYPYTFGGGTKLEIK | | | | |
| SEQ ID | LCDR1 Sequence | SEQ ID | LCDR2 Sequence | SEQ ID | LCDR3 Sequence |
| 93 | HASQNINVWLG | 94 | KASNLHT | 95 | QQGOSYPYT |

Clone F

| SEQ ID | VH | | | | |
|---|---|---|---|---|---|
| 96 | EVQLQQSGAEFVRPGASVKLSCTASGENIKDSLMHWVKQRPEQGLEWIGWIDPEDDKTKYAPKFQDEAT | | | | |
| | LTADTSSNTAYLHLSSLTSEDTAIYYCARRELRGFAYWGQGTLVTVSA | | | | |
| SEQ ID | HCDR1 Sequence | SEQ ID | HCDR2 Sequence | SEQ ID | HCDR3 Sequence |
| 97 | GFNIKDSLMH | 98 | WIDPEDDKTKYAPKFQD | 99 | RFLRGFAY |
| SEQ ID | VL | | | | |
| 100 | DIVMTQSPSSLSVSAGEKVTMSCKSSQSLLNSGNQKNYLAWYQQKPGQPPKLLIYGASTRESGVPDRFT | | | | |
| | GSGSGTDFTLTISSVQAEDLAVYYCQNDHSYPYTFGGGTKLEIK | | | | |
| SEQ ID | LCDR1 Sequence | SEQ ID | LCDR2 Sequence | SEQ ID | LCDR3 Sequence |
| 101 | KSSQSLLNSGNQKNYLA | 102 | GASTRES | 103 | QNDHSYPYT |

Clone G

| SEQ ID | VH | | | | |
|---|---|---|---|---|---|
| 104 | QVQLQQPGPELVKPVASVKISCKTSDYAFSSSWMNWVKQRPGKGLEWIGWIYPGDGDTNYNGKFKGKAT | | | | |
| | LTADKSSSTAYMQLSSLTSEDSAVYFCARGRWGMDNWGQGTSVTVSS | | | | |
| SEQ ID | HCDR1 Sequence | SEQ ID | HCDR2 Sequence | SEQ ID | HCDR3 Sequence |
| 105 | DYAFSSSWMN | 106 | WIYPGDGDTNYNGKFKG | 107 | GRWGMIDN |
| SEQ ID | VL | | | | |
| 108 | DIVMTQSHKEMSTSVGDRVSITCKASQNVVTAVAWYQQKPGQSPKLLIYWASSRHTGVPDRFTGSGSGT | | | | |
| | DFTLTISNVQSEDLADYFCQQYSNYPFTFGSGTKLEIK | | | | |
| SEQ ID | LCDR1 Sequence | SEQ ID | LCDR2 Sequence | SEQ ID | LCDR3 Sequence |
| 109 | KASQNVVTAVA | 110 | WASSRHT | 111 | QQYSNYPFT |

Clone H

| SEQ ID | VH | | | | |
|---|---|---|---|---|---|
| 112 | QVPLHQSGPELIKPGASLKISCKASDSAENNSWMNWVRQRPGKGLEWIGWIYPGDDDTKYNGKEMGKAT | | | | |
| | LTADKSSGTAYMQLSSLTSEDSAVYFCARGRWGMDYWGQGTSVTVSS | | | | |
| SEQ ID | HCDR1 Sequence | SEQ ID | HCDR2 Sequence | SEQ ID | HCDR3 Sequence |
| 113 | DSAFNNSWMN | 114 | WIYPGDDDTKYNGKEMG | 115 | GRWGMDY |
| SEQ ID | VL | | | | |
| 116 | DIVMTQFHNEMSTSVGDRVNINCKASQNVVTAVAWYQQKPGQSPKLLIYWASTRHTGVPDRFTGSGSGT | | | | |
| | DFTLTISNVQSEDLADYFCQQYSNYPFTFGSGTKLEIK | | | | |
| SEQ ID | LCDR1 Sequence | SEQ ID | LCDR2 Sequence | SEQ ID | LCDR3 Sequence |
| 117 | KASQNVVTAVA | 118 | WASTRHT | 119 | QQYSNYPFT |

Clone I

| SEQ ID | VH | | | | |
|---|---|---|---|---|---|
| 120 | QVPLQQSGPELVKPGASVKISCKASDSAENNSWTIWVRQRPGKGLEWIGWIYPGDDDTKYNGKFMGKAT | | | | |
| | LTADKSSGTAYMQLSSLISEDSAVYFCARGRWGMDYWGQGTSVTVSS | | | | |
| SEQ ID | HCDR1 Sequence | SEQ ID | HCDR2 Sequence | SEQ ID | HCDR3 Sequence |
| 121 | DSAENNSWTI | 122 | WIYPGDDDTKYNGKEMG | 123 | GRWGMIDY |
| SEQ ID | VL | | | | |
| 124 | DIVMTQSRKEMSTSVGDRVSISCKASQNVVTAVAWYQQKPGQSPKLLIYWASTRHTGVPDRFTGSGSGT | | | | |
| | DFTLTISNVQSEDLADYFCQQYSNYPFTEGSGTELEIK | | | | |
| SEQ ID | LCDR1 Sequence | SEQ ID | LCDR2 Sequence | SEQ ID | LCDR3 Sequence |
| 125 | KASQNVVTAVA | 126 | WASTRHT | 127 | QQYSNYPFT |

### Example 2: Generation of Chimeric CD38 Binders

Chimeric IgM and IgG constructs were generated from anti-CD38 murine hybridoma clones in Example 1 using standard cloning protocols. In brief for IgM constructs, heavy chain variable regions (VH) and light chain variable regions (VL) of eight anti-CD38 murine hybridoma clones were cloned into an IgM format containing a modified J-chain comprising a CD3-binding single-chain variable fragment (scFv). The IgM antibodies are described in Table 3. The resulting constructs were expressed in Expi293 cells and purified according to the methods described in Keyt et al., Antibodies: 9:53, doi: 10.3390/antib9040053 (2020). Chimeric IgM antibodies were successfully expressed and assembled as pentamers with a J-chain for clones A, B, C, E, F, and H.

TABLE 3
Chimeric CD38 × CD3 IgM Antibodies

| Antibody Name | VH | VL | J chain with scFv |
|---|---|---|---|
| IgM A-1 | SEQ ID NO: 56 | SEQ ID NO: 60 | SEQ ID NO: 166 |
| IgM B-1 | SEQ ID NO: 64 | SEQ ID NO: 68 | SEQ ID NO: 166 |
| IgM C-1 | SEQ ID NO: 72 | SEQ ID NO: 76 | SEQ ID NO: 166 |
| IgM E-1 | SEQ ID NO: 88 | SEQ ID NO: 92 | SEQ ID NO: 166 |
| IgM F-1 | SEQ ID NO: 96 | SEQ ID NO: 100 | SEQ ID NO: 166 |
| IgM H-1 | SEQ ID NO: 112 | SEQ ID NO: 116 | SEQ ID NO: 166 |

Chimeric human IgG1 CD38 binders were generated by cloning the entire murine VH and VL regions of the clones in Example 1 into human IgG1 format according to standard cloning protocols. Human IgG constructs were synthesized, expressed, and purified through commercial vendors. The subset of chimeric IgG antibodies used in the examples below are described in Table 4.

TABLE 4
Chimeric IgG Antibodies

| Antibody Name | VH | VL |
|---|---|---|
| IgG B-1 | SEQ ID NO: 64 | SEQ ID NO: 68 |
| IgG E-1 | SEQ ID NO: 88 | SEQ ID NO: 92 |

### Example 3: Binding Specificity of Chimeric CD38×CD3 IgM Antibodies

Binding specificities of the chimeric CD38×CD3 IgM antibodies generated in Example 2 for human and cynomolgus (cyno) CD38 were measured in ELISA assays as follows. 96-well white polystyrene ELISA plates (Pierce 15042) were coated with recombinant human or cyno CD38 extracellular domain proteins from ACROBiosystems and incubated overnight at 4° C. Plates were then washed five times with 0.05% phosphate buffered saline (PBS)-polysorbate sold under the trademark TWEEN® (PBS-TWEEN®) and blocked with 2% bovine serum albumin (BSA)-PBS. After blocking, 100 μL of serial dilutions of chimeric CD38×CD3 IgM antibodies, standards, and controls were added to each well of the plates and incubated at room temperature for 2 hours. The plates were then washed 10 times with 0.05% PBS-TWEEN® and incubated with horseradish peroxidase (HRP)-conjugated mouse anti-human Kappa secondary antibody (SouthernBiotech, clone SB81a) for 30 minutes. After 10 final washes using 0.05% PBS-TWEEN®, the plates were read using SUPERSIGNAL™ chemiluminescent substrate (ThermoFisher, 37070). Luminescent data was collected on an ENVISION® plate reader (PERKINELMER®) and analyzed with GRAPHPAD PRISM® using a 4-parameter logistic model.

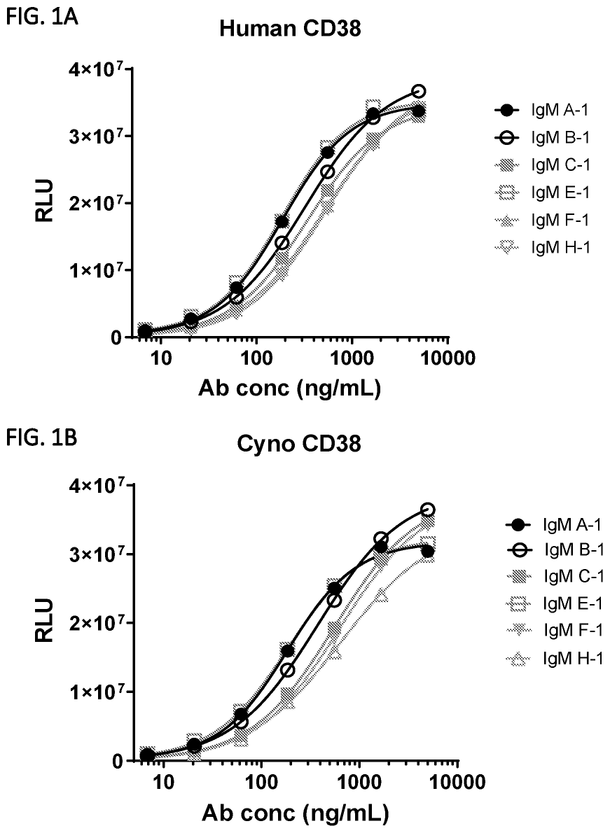

FIGS. 1A-1B show binding of chimeric CD38×CD3 IgM antibodies to human CD38 (FIG. 1A) and cyno CD38 (FIG. 1B). Data is presented in relative luminescence units (RLU) for the binding of chimeric CD38×CD3 IgM antibodies (IgM A-1, IgM B-1, IgM C-1, IgM E-1, IgM F-1, and IgM H-1).

All chimeric CD38×CD3 IgM antibodies bound to human and cyno CD38 in a dose-response manner with similar affinities.

In addition, binding to cell surface human CD38 was assessed using the myeloma cell line, NCI-H929 (H929), and flow cytometry analysis. In brief, H929 cells were stained with serial dilutions of chimeric CD38×CD3 IgM antibodies for 30 minutes at 4° C. Cells were washed twice and stained for 30 minutes at 4° C. with ALEXA FLUOR® 488 (AF488)-conjugated mouse anti-human Kappa secondary antibody (SouthernBiotech, clone SB81a). Cells were then washed twice, resuspended in fluorescence-activated cell sorting (FACS) stain buffer, and measured using flow cytometry.

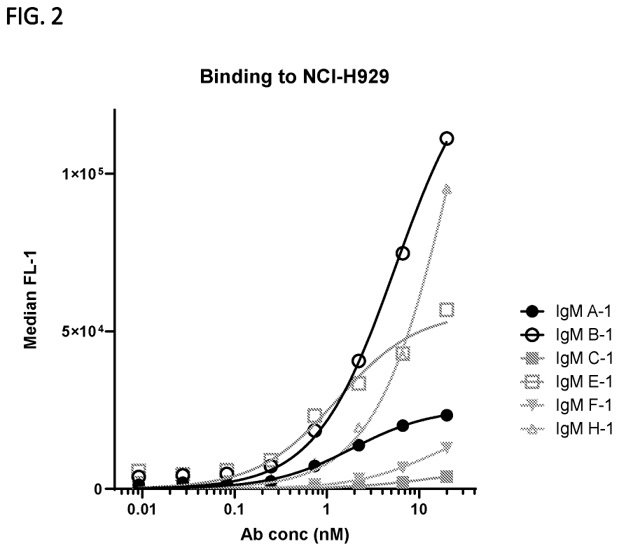

FIG. 2 shows binding of chimeric CD38×CD3 IgM antibodies to NCI-H929 cells measured by flow cytometry. Data is presented as median fluorescence intensity (FL-1 parameter for detection of AF488) for binding of chimeric CD38×CD3 IgM antibodies (IgM A-1, IgM B-1, IgM C-1, IgM E-1, IgM F-1, and IgM H-1).

Chimeric CD38×CD3 IgM antibodies IgM B-1, IgM E-1, and IgM H-1 had particularly good activity in binding assays.

### Example 4: Testing Chimeric CD38×CD3 IgM Antibodies in Functional Assays

T cell-dependent cellular cytotoxicity (TDCC) activities of chimeric CD38×CD3 IgM antibodies were tested in an in vitro co-culture assay. In brief, peripheral blood mononuclear cells (PBMCs) or CD3+ pan T cells were co-cultured with luciferase-tagged H929 and OPM2 myeloma cell lines at different effector to target ratios (1:1, 5:1 and 20:1). Cell cytotoxicity was read at 24 hours, 48 hours, 72 hours and/or 96 hours using luciferase assay substrate from PROMEGA® and converted to a maximum percentage of cells killed (Max Killing %). Representative data are shown in Tables 5-8 below, along with measured EC₅₀ values.

TABLE 5
Functional Properties of Chimeric CD38 × CD3
IgM Antibodies on Co-Culture of CD3+ T
Cells and Luciferase-Tagged H929 Cells (1:1)

| | 24 hours | | 48 hours | |
|---|---|---|---|---|
| | Max | EC50 | Max | EC50 |
| Antibody | Killing % | (pM) | Killing % | (pM) |
| IgM A-1 | 26 | 143 | 100 | 60 |
| IgM B-1 | 37 | 184 | 100 | 34 |
| IgM C-1 | 51 | 200 | 100 | 69 |
| IgM E-1 | 32 | 59 | 100 | 34 |
| IgM F-1 | >20 | >2500 | 100 | 1882 |
| IgM H-1 | >30 | >2500 | 100 | 333 |

TABLE 6
Functional Properties of Chimeric CD38 × CD3
IgM Antibodies on Co-Culture of PBMC and
Luciferase-Tagged H929 Cells (1:1)

| | 24 hours | | 48 hours | |
|---|---|---|---|---|
| | Max | EC50 | Max | EC50 |
| Antibody | Killing % | (pM) | Killing % | (pM) |
| IgM A-1 | 36 | 69 | 93 | 40 |
| IgM B-1 | 42 | 115 | 97 | 33 |
| IgM C-1 | 51 | 113 | 98 | 45 |
| IgM E-1 | 42 | 26 | 99 | 24 |
| IgM F-1 | 42 | 1867 | 100 | 1058 |
| IgM H-1 | 31 | 496 | 90 | 192 |

TABLE 7
Functional Properties of Chimeric CD38 × CD3
IgM Antibodies on Co-Culture of CD3+ T Cells and
Luciferase-Tagged OPM2 Cells (1:1)

| | Max | EC50 | Max | EC50 |
|---|---|---|---|---|
| Antibody | Killing % | (pM) | Killing % | (pM) |
| | 24 hours | | 48 hours | |
| IgM A-1 | 11 | 12 | 19 | 2.2 |
| IgM B-1 | 10 | 25 | 20 | 2.2 |
| IgM C-1 | 42 | 91 | 80 | 145 |
| IgM E-1 | >10 | >2500 | >20 | >2500 |
| IgM F-1 | 21 | 2265 | 23 | 514 |
| IgM H-1 | 30 | 1397 | >30 | >2500 |
| | 72 hours | | 96 hours | |
| IgM A-1 | 23 | 7.4 | 24 | 5.4 |
| IgM B-1 | 32 | 50 | 24 | 1.8 |
| IgM C-1 | 100 | 156 | 100 | 156 |
| IgM E-1 | >25 | >2500 | 48 | 696 |
| IgM F-1 | 38 | 533 | 56 | 1608 |
| IgM H-1 | >30 | >2500 | >50 | >2500 |

TABLE 8
Functional Properties of Chimeric CD38 × CD3
IgM Antibodies on Co-Culture of PBMC and
Luciferase-Tagged OPM2 Cells (1:1)

| | Max | EC50 | Max | EC50 |
|---|---|---|---|---|
| Antibody | Killing % | (pM) | Killing % | (pM) |
| | 24 hours | | 48 hours | |
| IgM A-1 | 15 | 8.1 | 26 | 4.3 |
| IgM B-1 | 15 | 7.2 | 25 | 3.8 |
| IgM C-1 | 30 | 23 | 74 | 28 |
| IgM E-1 | 7 | 8 | 18 | 4 |
| IgM F-1 | >20 | >2500 | 30 | 651 |
| IgM H-1 | 11 | 1603 | 35 | 1302 |
| | 72 hours | | 96 hours | |
| IgM A-1 | 33 | 4.2 | 32 | 6.8 |
| IgM B-1 | 23 | 2.3 | 29 | 3.4 |
| IgM C-1 | 100 | 30 | 100 | 19 |
| IgM E-1 | 23 | 5.7 | 26 | 7 |
| IgM F-1 | >35 | >2500 | >35 | >2500 |
| IgM H-1 | >32 | >2500 | >32 | >2500 |

Chimeric CD38×CD3 IgM antibodies IgM B-1 and IgM E-1 had particularly good activity in TDCC assays.

### Example 5: Humanization of CD38 Antibodies

Clones B and E were chosen for humanization based on the activity of the corresponding chimeric CD38×CD3 IgM antibodies in binding and TDCC assays. Additionally, known anti-CD38 antibodies OKT10 (VH-SEQ ID NO: 162, VL-SEQ ID NO: 163) and HB7 (VH-SEQ ID NO: 164, VL-SEQ ID NO: 165) were humanized. Human antibodies from the RCSB protein data bank (available on World Wide Web at rcsb.org) were chosen as framework acceptors: 4KQ3 for Clone B; 5115 for Clone E; 3MOD, 5FUZ, or 5K59 for OKT10; and 5HYS, 5SX4, or 4R7D for HB7. Complementarity determining regions (CDRs) of the murine hybridoma clones were directly grafted to the human framework acceptors. Some sequences were further designed to remove potential deamidation sites or other alterations. The resulting sequences for the clones are shown in Table 9 and for OKT10 and HB7 are shown in Table 10. The humanized VH and VL sequences were then cloned into human IgG and IgM formats according to standard cloning protocols. A subset was used for additional experiments and are described in Table 11.

TABLE 9
Humanized Sequences of Hybridoma Clones
Humanized Clone B

| SEQ ID | BVH1 | | | | |
|---|---|---|---|---|---|
| 128 | QVQLVQSGAEVKKPGSSVKVSCKASGYTFTSYNMHWVRQAPGQGLEWMGYIYPGNGGTNYNQKFKGRVT | | | | |
| | ITADESTSTAYMELSSLRSEDTAVYYCARGASMITTGAWFAYWGQGTLVTVSS | | | | |
| SEQ ID | HCDR1 Sequence | SEQ ID | HCDR2 Sequence | SEQ ID | HCDR3 Sequence |
| 65 | GYTFTSYNMH | 66 | YIYPGNGGTNYNQKEKG | 67 | GASMITTGAWFAY |
| SEQ ID | BVH2 | | | | |
| 129 | QVQLVQSGAEVKKPGSSVKVSCKASGYTFTSYNMHWVRQAPGQGLEWMGYIYPGNGGTNYNQKEKGRVT | | | | |
| | ITADTSTSTAYMELSSLRSEDTAVYYCARGASMITTGAWFAYWGQGTLVTVSS | | | | |
| SEQ ID | HCDR1 Sequence | SEQ ID | HCDR2 Sequence | SEQ ID | HCDR3 Sequence |
| 65 | GYTFTSYNMH | 66 | YIYPGNGGTNYNQKFKG | 67 | GASMITTGAWFAY |
| SEQ ID | BVH3 | | | | |
| 130 | QVQLVQSGAEVKKPGSSVKVSCKASGYTFTSYNMHWVRQAPGQGLEWMGYIYPGSGGTNYNQKFKGRVT | | | | |
| | ITADESTSTAYMELSSLRSEDTAVYYCARGASMITTGAWFAYWGQGTLVTVSS | | | | |
| SEQ ID | HCDR1 Sequence | SEQ ID | HCDR2 Sequence | SEQ ID | HCDR3 Sequence |
| 65 | GYTFTSYNMH | 134 | YIYPGSGGTNYNQKFKG | 67 | GASMITTGAWFAY |
| SEQ ID | BVH4 | | | | |
| 131 | QVQLVQSGAEVKKPGSSVKVSCKASGYTFTSYNMHWVRQAPGQGLEWMGYIYPGNGGTSYNQKFKGRVT | | | | |
| | ITADESTSTAYMELSSLRSEDTAVYYCARGASMITTGAWFAYWGQGTLVTVSS | | | | |
| SEQ ID | HCDR1 Sequence | SEQ ID | HCDR2 Sequence | SEQ ID | HCDR3 Sequence |
| 65 | GYTFTSYNMH | 182 | YIYPGNGGTSYNQKFKG | 67 | GASMITTGAWFAY |
| SEQ ID | BVH5 | | | | |
| 132 | QVQLVQSGAEVKKPGSSVKVSCKASGYTFTSYNMHWVRQAPGQGLEWMGYIYPGSGGTSYNQKFKGRVT | | | | |
| | ITADESTSTAYMELSSLRSEDTAVYYCARGASMITTGAWFAYWGQGTLVTVSS | | | | |
| SEQ ID | HCDR1 Sequence | SEQ ID | HCDR2 Sequence | SEQ ID | HCDR3 Sequence |
| 65 | GYTFTSYNMH | 135 | YIYPGSGGTSYNQKFKG | 67 | GASMITTGAWFAY |
| SEQ ID | BVL1 | | | | |
| 133 | DIQMTQSPSSVSASVGDRVTITCRASESLDTYGNSFMYWYQQKPGKAPKLLIYLASSLESGVPSRESGS | | | | |
| | GSGTDFTLTISSLQPEDFATYYCHQNHEDPWTFGQGTKVEIK | | | | |
| SEQ ID | LCDR1 Sequence | SEQ ID | LCDR2 Sequence | SEQ ID | LCDR3 Sequence |
| 69 | RASESLDTYGNSEMY | 70 | LASSLES | 71 | HONHEDPWT |

Humanized Clone E

| SEQ ID | EVH1 | | | | |
|---|---|---|---|---|---|
| 136 | QVQLVQSGAEVKKPGSSVKVSCKASGYTFTNHWIEWVRQAPGQGLEWMGDIFPGGGYTDYNEKFKGRVT | | | | |
| | ITADESTSTAYMELSSLRSEDTAVYYCARGRDLAMDYWGQGTLVTVSS | | | | |
| SEQ ID | HCDR1 Sequence | SEQ ID | HCDR2 Sequence | SEQ ID | HCDR3 Sequence |
| 89 | GYTFTNHWIE | 90 | DIFPGGGYTDYNEKFKG | 91 | GRDLAMDY |
| SEQ ID | EVH2 | | | | |
| 137 | QVQLVQSGAEVKKPGSSVKVSCKASGYTFTNHWIEWVRQAPGQGLEWIGDIFPGGGYTDYNEKEKGRAT | | | | |
| | ITADESTSTAYMELSSLRSEDTAVYYCARGRDLAMDYWGQGTLVTVSS | | | | |
| SEQ ID | HCDR1 Sequence | SEQ ID | HCDR2 Sequence | SEQ ID | HCDR3 Sequence |
| 89 | GYTFTNHWIE | 90 | DIFPGGGYTDYNEKFKG | 91 | GRDLAMDY |
| SEQ ID | EVL1 | | | | |
| 138 | DIQMTQSPSSLSASVGDRVTITCHASQNINVWLGWYQQKPGKAPKLLIYKASNLHTGVPSRFSGSGSGT | | | | |
| | DFTLTISSLQPEDFATYYCQQGQSYPYTFGQGTKVEIK | | | | |
| SEQ ID | LCDR1 Sequence | SEQ ID | LCDR2 Sequence | SEQ ID | LCDR3 Sequence |
| 93 | HASQNINVWLG | 94 | KASNLHT | 95 | QQGQSYPYT |
| SEQ ID | EVL2 | | | | |
| 139 | DIQMTQSPSSLSASVGDRVTITCRASQNINVWLGWYQQKPGKAPKLLIYKASNLHTGVPSRFSGSGSGT | | | | |
| | DFTLTISSLQPEDFATYYCQQGQSYPYTFGQGTKVEIK | | | | |
| SEQ ID | LCDR1 Sequence | SEQ ID | LCDR2 Sequence | SEQ ID | LCDR3 Sequence |
| 143 | RASQNINVWLG | 94 | KASNLHT | 95 | QQGQSYPYT |
| SEQ ID | EVL3 | | | | |
| 140 | DIQMTQSPSSLSASVGDRVTITCSASQNINVWLGWYQQKPGKAPKLLIYKASNLHTGVPSRFSGSGSGT | | | | |
| | DFTLTISSLQPEDFATYYCQQGQSYPYTFGQGTKVEIK | | | | |
| SEQ ID | LCDR1 Sequence | SEQ ID | LCDR2 Sequence | SEQ ID | LCDR3 Sequence |
| 144 | SASQNINVWLG | 94 | KASNLHT | 95 | QQGQSYPYT |
| SEQ ID | EVL4 | | | | |
| 141 | DIQMTQSPSSLSASVGDRVTITCGASQNINVWLGWYQQKPGKAPKLLIYKASNLHTGVPSRESGSGSGT | | | | |
| | DFTLTISSLQPEDFATYYCQQGQSYPYTFGQGTKVEIK | | | | |
| SEQ ID | LCDR1 Sequence | SEQ ID | LCDR2 Sequence | SEQ ID | LCDR3 Sequence |
| 145 | GASQNINVWLG | 94 | KASNLHT | 95 | QQGQSYPYT |
| SEQ ID | EVL5 | | | | |
| 142 | DIQMTQSPSSLSASVGDRVTITCEASQNINVWLGWYQQKPGKAPKLLIYKASNLHTGVPSRFSGSGSGT | | | | |
| | DFTLTISSLQPEDFATYYCQQGQSYPYTFGQGTKVEIK | | | | |
| SEQ ID | LCDR1 Sequence | SEQ ID | LCDR2 Sequence | SEQ ID | LCDR3 Sequence |
| 146 | EASQNINVWLG | 94 | KASNLHT | 95 | QQGQSYPYT |

TABLE 10
Other Humanized CD38 Binders
Parental OKT10

| | |
|---|---|
| SEQ ID | Parental OKT10 VH |
| 178 | EVKLQESGGGLVQPGGSLKLSCAASGFDFSRSWMNWVRQAPGKGLEWIGEINPDSSTINYTTSLKDKFI |
| | ISRDNAKNTLYLQMTKVRSEDTALYYCARYGNWFPYWGQGTLVTVSA |
| SEQ ID | Parental OKT10 VL |
| 179 | DIVMTQSPKIMPTSVGDRVSVTCKASQNVDTNVAWYQQKPGQSPKALIYSASYRYSGVPDRFTGSGSGT |
| | DFTLTITNVQSEDLAEYFCQQYDSYPLTFGAGTKLDLK |

Humanized OKT10

| | |
|---|---|
| SEQ ID | OKT10VH1 |
| 147 | QITLKESGPTLVKPTQTLTLTCTASGFDFSRSWMNWIRQPPGKALEWLAEINPDSSTINYTTSLKDRFT |
| | ISRDISKNQVVLTMTNMDPVDTATYYCARYGNWFPYWGQGTLVTVSS |
| SEQ ID | OKT10VH2 |
| 148 | EVQLLESGGGLVQPGGSLRLSCAASGFDFSRSWMNWVRQAPGKGLEWVSEINPDSSTINYATSLKDRFT |
| | ISRDNSKNTLYLQMNSLRAEDTAVYYCARYGNWFPYWGQGTLVTVSS |
| SEQ ID | OKT10VH3 |
| 149 | QLQLQESGPGLVKPSETLSLTCTASGFDFSRSWMNWIRQPPGKGLEWIGEINPDSSTINYATSLKDRFT |
| | ISRDTSKNQLSLKLSSVTAADTAVYYCARYGNWFPYWGQGTLVTVSS |
| SEQ ID | OKT10VL1 |
| 150 | AIQLTQSPSSLSASVGDRVTITCKASQNVDTNVAWYQQKPGKAPKALIYSASYRYSGVPSRFSGSGSGT |
| | DFTLTISSLQPEDFATYYCQQYDSYPLTFGQGTKVEIK |
| SEQ ID | OKT10VL2 |
| 151 | DIQMTQSPSSLSASVGDRVTITCKASQNVDTNVAWYQQKPGKAPKALIYSASYRYSGVPSRFSGSGSGT |
| | DETLTISSLQPEDFATYYCQQYDSYPLTFGQGTKVEIK |
| SEQ ID | OKT10VL3 |
| 152 | DIQMTQSPSSLSASVGDRVTITCRASQNVDTNVAWYQQKPGKAPKALIYSASYRYSGVPSRFSGSGSGT |
| | DFTLTISSLQPEDFATYYCQQYDSYPLTFGQGTKVEIK |
| SEQ ID | OKT10VL4 |
| 153 | DIQMTQSPSSLSASVGDRVTITCSASQNVDINVAWYQQKPGKAPKALIYSASYRYSGVPSRFSGSGSGT |
| | DFTLTISSLQPEDFATYYCQQYDSYPLTFGQGTKVEIK |
| SEQ ID | OKT10VL5 |
| 154 | DIQMTQSPSSLSASVGDRVTITCGASQNVDINVAWYQQKPGKAPKALIYSASYRYSGVPSRFSGSGSGT |
| | DETLTISSLQPEDFATYYCQQYDSYPLTFGQGTKVEIK |
| SEQ ID | OKT10VL6 |
| 155 | DIQMTQSPSSLSASVGDRVTITCEASQNVDTNVAWYQQKPGKAPKALIYSASYRYSGVPSRFSGSGSGT |
| | DFTLTISSLQPEDFATYYCQQYDSYPLTFGQGTKVEIK |

Parental HB7

| | |
|---|---|
| SEQ ID | Parental HB7 VH |
| 180 | TLVQLKQSGPSLVQPSQRLSITCTVSGFSLISYGVHWVRQSPGKGLEWLGVIWRGGSTDYNAAFMSRLS |
| | ITKDNSKSQVFFKMNSLQADDTAIYFCAKTLITTGYAMDYWGQGTSVTVSS |
| SEQ ID | Parental HB7 VL |
| 181 | IQMTQSSSSFSVSLGDRVTITCKASEDIYNRLAWYQQKPGNAPRLLISGATSLETGVPSRFSGSGSGKD |
| | YTLSITSLQTEDVATYYCQQYWSTPTFGGGTKLEIK |

Humanized HB7

| | |
|---|---|
| SEQ ID | HB7VH1 |
| 156 | EVQLVESGGGLVQPGGSLRLSCAVSGFSLISYGVHWVRQAPGKGLEWLGVIWRGGSTDYNAAFMSRLTI |
| | SKDNSKNTVYLQMNSLRAEDTAVYYCAKTLITTGYAMDYWGQGTLVTVSS |
| SEQ ID | HB7VH2 |
| 157 | QVQLQESGPGLVKPSETLSLTCTVSGFSLISYGVHWVRQPPGKGLEWLGVIWRGGSTDYNAAFMSRLTI |
| | SKDISKNQVSLKLSSVTAADTAVYYCAKTLITTGYAMDYWGQGTLVTVSS |
| SEQ ID | HB7VH3 |
| 158 | QVQLQESGPGLVKPSDTLSLTCAVSGFSLISYGVHWVRQPPGKGLEWLGVIWRGGSTDYNAAFMSRLTM |
| | SKDTSKNQVSLKLSSVTAVDTAVYYCAKTLITTGYAMDYWGQGTLVTVSS |
| SEQ ID | HB7VL1 |
| 159 | DIQMTQSPSSLSASVGDRVTITCKASEDIYNRLAWYQQKPGKAPKLLISGATSLETGVPSRFSGSGSGT |
| | DYTLTISSLQPEDFATYYCQQYWSTPTEGQGTKVEIK |
| SEQ ID | HB7VL2 |
| 160 | DIQMTQSPSSLSASVGDRVTITCKASEDIYNRLAWYQQKPGKAPKLLISGATSLETGVPSRFSGSGSGT |
| | DYTFTISSLQPEDIATYYCQQYWSTPTFGQGTKVEIK |
| SEQ ID | HB7VL3 |
| 161 | EIVLTQSPDFQSVTPKEKVTITCKASEDIYNRLAWYQQKPDQSPKLLISGATSLETGVPSRFSGSGSGT |
| | DYTLTINSLEAEDAATYYCQQYWSTPTFGQGTKVEIK |

TABLE 11
IgM and IgG Antibodies

| Antibody | | | | |
|---|---|---|---|---|
| Name | Format | VH | VL | J chain with scFv |
| IgG OKT10-1 | IgG | SEQ ID NO: 178 | SEQ ID NO: 179 | N/A |
| IgG OKT10-2 | IgG | SEQ ID NO: 148 | SEQ ID NO: 151 | N/A |
| IgM OKT10-2 | IgM | SEQ ID NO: 148 | SEQ ID NO: 151 | SEQ ID NO: 167 |
| IgM OKT10-4 | IgM | SEQ ID NO: 148 | SEQ ID NO: 151 | SEQ ID NO: 169 |
| IgG B-2 | IgG | SEQ ID NO: 128 | SEQ ID NO: 133 | N/A |
| IgM B-2 | IgM | SEQ ID NO: 128 | SEQ ID NO: 133 | SEQ ID NO: 167 |
| IgM B-3 | IgM | SEQ ID NO: 128 | SEQ ID NO: 133 | SEQ ID NO: 168 |
| IgM B-4 | IgM | SEQ ID NO: 128 | SEQ ID NO: 133 | SEQ ID NO: 169 |
| IgM B-5 | IgM | SEQ ID NO: 128 | SEQ ID NO: 133 | SEQ ID NO: 170 |
| IgG E-2 | IgG | SEQ ID NO: 136 | SEQ ID NO: 138 | N/A |
| IgM E-2 | IgM | SEQ ID NO: 136 | SEQ ID NO: 138 | SEQ ID NO: 167 |
| IgM E-3 | IgM | SEQ ID NO: 136 | SEQ ID NO: 138 | SEQ ID NO: 168 |
| IgM E-4 | IgM | SEQ ID NO: 136 | SEQ ID NO: 138 | SEQ ID NO: 169 |
| IgM E-5 | IgM | SEQ ID NO: 136 | SEQ ID NO: 138 | SEQ ID NO: 170 |

### Example 6: Biolayer Interferometry Affinity Measurement Assay of Humanized IgG Antibodies

Binding affinities of the chimeric IgG and humanized IgG variants described in Example 5 to recombinant human CD38 protein (ACROBiosystems, Cat #CD8-H5224) were determined by biolayer interferometry (BLI) on an OCTET® 384 system (Sartorius/Forte Biosciences) using anti-human IgG FC biosensors (Sartorius, Part #18-5004) and standard protocols. Samples and buffer were applied in 384-well plates. After an initial baseline calibration for 60 seconds, sensors were loaded with 20 nM of IgG antibodies. The biosensors were dipped into buffer to reach a baseline value and incubated for 600 seconds with 2-fold serial diluted human CD38 protein starting at 100 nM for association, followed by 900 seconds in buffer for dissociation. Affinities were analyzed by ForteBio Data Analysis software 9.0 using 1:1 global fit model, and the resulting binding properties are shown below in Table 12.

TABLE 12
Binding Affinities of Chimeric IgG and Humanized IgG Variants
with Recombinant Human CD38 Protein

| Antibody | K_{D} (M) | Kₒₙ (1/Ms) | K_{dis} (1/s) |
|---|---|---|---|
| IgG OKT10-1 | 5.97 × 10⁻⁹ | 3.44 × 10⁵ | 2.06 × 10⁻³ |
| IgG OKT10-2 | 2.83 × 10⁻⁸ | 4 × 10⁵ | 1.13 × 10⁻² |
| IgG B-1 | 5.26 × 10⁻¹⁰ | 1.39 × 10⁵ | 7.32 × 10⁻⁵ |
| IgG B-2 | 8 × 10⁻¹⁰ | 2.81 × 10⁵ | 2.25 × 10⁻⁴ |
| IgG E-1 | 1.99 × 10⁻⁹ | 5.87 × 10⁵ | 1.17 × 10⁻³ |
| IgG E-2 | 7.62 × 10⁻⁹ | 4.96 × 10⁵ | 3.78 × 10⁻³ |

Humanized IgG variants have similar binding properties compared to their chimeric IgG counterparts.

### Example 7: Humanized CD38×CD3 IgMs with Various CD3 Binding Domains

Bispecific IgMs with different CD38 and CD3 binding domains were tested in a T cell redirecting cytotoxicity assay using CD3+ pan T cells and luciferase (Luc)-tagged H929 or OPM2 myeloma cell lines, at an effector to target ratio of 10:1 or 1:1. Cell cytotoxicity was evaluated as described in Example 4 at 48 hours (H929 cells) or 72 hours (OPM2 cells) using luciferase assay substrate from PROMEGA®. Results are shown in Table 13 (H929 cells) and Table 14 (OPM2 cells).

TABLE 13
Cytotoxicity of Humanized CD38 × CD3 IgMs on H929 Cells

| | CD3+ T Cells: | | CD3+ T Cells: | |
|---|---|---|---|---|
| | H929-Luc (1:1) | | H929-Luc (10:1) | |
| | Max | EC50 | Max | EC50 |
| Antibody | Killing % | (pM) | Killing % | (pM) |
| IgM B-2 | 98 | 60 | 99 | 45 |
| IgM B-3 | 92 | 101 | 98 | 90 |
| IgM B-4 | 100 | 38 | 100 | 57 |
| IgM B-5 | 100 | 58 | 100 | 71 |
| IgM E-2 | 99 | 25 | 99 | 13 |
| IgM E-3 | 98 | 35 | 99 | 25 |
| IgM E-4 | 100 | 25 | 100 | 18 |
| IgM E-5 | ~100 | 28 | 100 | 23 |

TABLE 14
Cytotoxicity of Humanized CD38 × CD3 IgMs on OPM2 Cells

| | CD3+ T Cells: | | CD3+ T Cells: | |
|---|---|---|---|---|
| | OPM2-Luc (1:1) | | OPM2-Luc (10:1) | |
| | Max | EC50 | Max | EC50 |
| Antibody | Killing % | (pM) | Killing % | (pM) |
| IgM B-2 | 51 | 0.66 | 86 | 7 |
| IgM B-3 | 39 | 0.56 | 84 | 36 |
| IgM B-4 | 64 | 2.1 | ~100 | 12 |
| IgM B-5 | 94 | 16 | ~100 | 6.8 |
| IgM E-2 | 83 | 9.8 | 98 | 4.4 |
| IgM E-3 | 52 | 17 | ~100 | 12 |
| IgM E-4 | 88 | 4.7 | 100 | 3.4 |
| IgM E-5 | 100 | 12 | 100 | 2.3 |

These data show that the humanized CD38×CD3 IgM antibodies achieved close to complete killing of H929 and OPM2 cells with similar EC50 values.

### Example 8: Specificity of Humanized CD38×CD3 IgM Antibodies

The specificities of humanized CD38×CD3 IgM antibodies were measured in ELISA assays as follows. 96-well white polystyrene ELISA plates (PIERCE™, 15042) were coated with 100 μL per well of 1 μg/mL recombinant human CD38 (ACROBiosystems, CD8-H5224), mouse CD38 (ACROBiosystems, CD8-M5223), pig CD38 (BPS Bioscience, 101019), dog CD38 (BPS Bioscience, Cat #100955), rabbit CD38 (SinoBiological, 65003-T08H) or human CD157 protein (SinoBiological, 10060-H08H) overnight at 4° C. Plates were then washed with 0.05% PBS-polysorbate sold under the trademark TWEEN® (PBS-TWEEN®) and blocked with 2% BSA-PBS. After blocking, 100 μL of serial dilutions of humanized CD38×CD3 IgM antibodies and controls were added to the wells and incubated at room temperature for 2 hours. The plates were then washed and incubated with HRP-conjugated mouse anti-human Kappa antibody (SouthernBiotech, 9230-05. 1:6000 diluted in 2% BSA-PBS) for 30 minutes. After final washes, the plates were read using SUPERSIGNAL™ chemiluminescent substrate (ThermoFisher, 37070). Luminescent data were collected on an ENVISION® plate reader (PERKINELMER®) and analyzed with GRAPHPAD PRISM® using a 4-parameter logistic model.

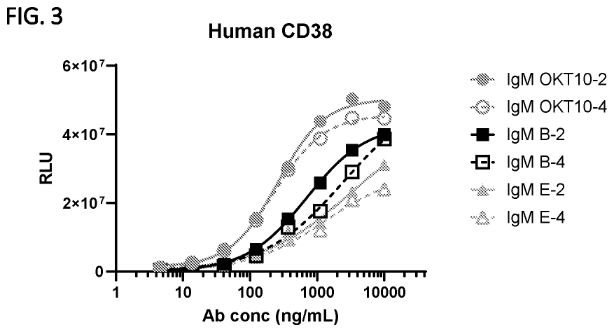

FIG. 3 shows binding of humanized CD38×CD3 IgM antibodies to human CD38 at different antibody concentrations measured by ELISA. Data is presented in relative luminescence units (RLU) for humanized CD38×CD3 IgM antibodies (IgM B-2, IgM B-4, IgM E-2, IgM E-4, IgM OKT10-2, and IgM OKT10-4). These antibodies did not bind mouse CD38 or human CD157.

In addition, a subset of CD38×CD3 IgM antibodies (IgM B-2, IgM E-2, and IgM OKT10-2) were assessed for binding to human CD38, pig CD38, dog CD38, or rabbit CD38. Binding was similar to that seen in FIG. 3 for human CD38, but virtually no binding was detected for pig CD38, dog CD38, or rabbit CD38 (data not shown).

### Example 9: Pharmacokinetics of CD38×CD3 IgM Antibodies

Pharmacokinetic parameters were measured for various CD38×CD3 IgM antibodies in an in vivo mouse model as follows. Balb/c mice were injected with 5 mg/kg of IgM B-2, IgM E-2, and IgM OKT10-2 via intravenous infusion. Blood samples were collected at 10 or 12 time points total for each antibody, with 2 mice per time point. Each mouse was bled once through the facial vein (100 μL) and then another time by terminal cardiac puncture (max obtainable, ˜500 μL). A standard ELISA assay was used to measure the serum concentration of each antibody in the blood at each time point. Quality metrics were verified on all ELISAs, and PK parameters, including t_{1/2}, clearance (CL), area under the concentration curve (AUC), and maximum concentration (Cmax) were derived using standard curve fitting techniques (Win Non Lin, Phoenix Software). A plot of concentrations over time is shown in FIG. 4. PK parameters are presented in Table 15.

TABLE 15
PK parameters

| | t1/2 | Cmax | AUC | Cl |
|---|---|---|---|---|
| Antibody | (hrs) | (μg/mL) | (day*μg/mL) | (mL/day/kg) |
| IgM B-2 | 27.3 | 127 | 125 | 40 |
| IgM E-2 | 9.0 | 110 | 37 | 133 |
| IgM OKT10-2 | 5.3 | 95 | 26 | 192 |

### Example 10: CD38×CD3 IgM Shows Similar TDCC Potency as CD38×CD3 IgG with Lower Cytokine Release

T cell-dependent cellular cytotoxicity (TDCC) activity of the humanized CD38×CD3 IgM antibody, IgM B-2, was tested in an in vitro co-culture assay as explained in Example 4. Three IgG-based molecules were generated for comparison: CD38 IgG Y comprising SEQ ID NOs: 174 and 175, CD38 IgG Z comprising SEQ ID NOs: 176 and 177, and CD38×CD3 IgG comprising SEQ ID NOs 171-173 and tested in the assay, which also detects antibody-dependent cellular cytotoxicity (i.e., the primary mechanism of CD38 IgG Y and CD38 IgG Z). The three comparator IgG molecules were synthesized, expressed, and purified through commercial vendors. CD38 IgG Y and CD38 IgG Z were generated using standard protocols. CD38×CD3 IgG was generated using methods described in Moore, et al., Methods, 2019, 154:38-50.

Supernatants from the assays were collected at various time points and TNF-α and IFN-γ levels in the supernatants were measured using BD Cytometric Bead Assay Human Th1/Th2 Cytokine Kit II (BD 551809) according to manufacturer's protocol. The results were then analyzed with GraphPad Prism.

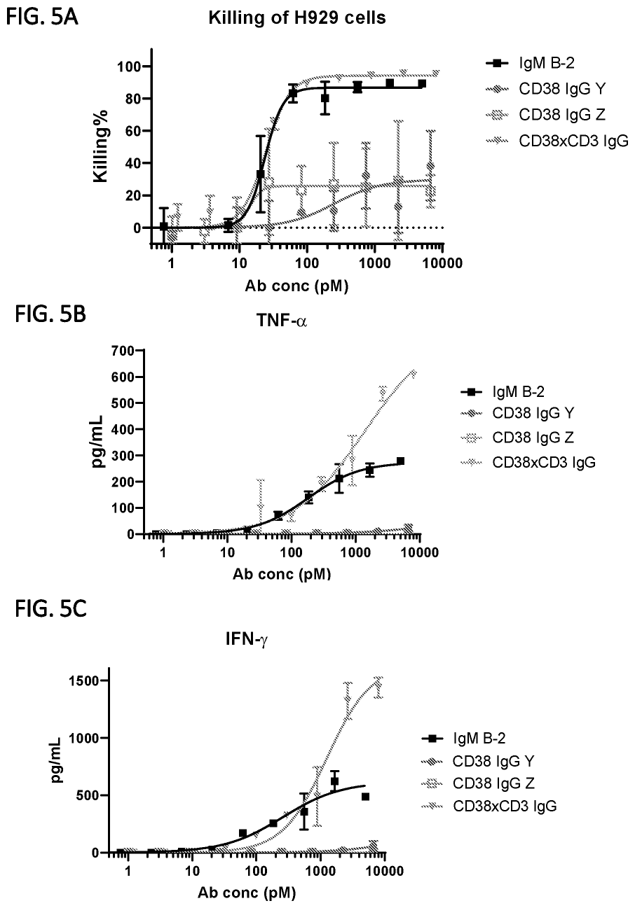

The resulting cytotoxicity curves for CD38 IgG Y, CD38 IgG Z, CD38×CD3 IgG, and IgM B-2 are shown in FIGS. 5A-5C, and maximum killing percentage, TNF-α and IFN-γ and killing EC50 values are shown in Table 16. FIG. 5A shows the dose response curve for killing of H929 cells (Killing %) with increasing concentration of CD38 IgG Y, CD38 IgG Z, CD38×CD3 IgG, and IgM B-2. FIG. 5B shows TNF-α levels that resulted following treatment with increasing concentration of CD38 IgG Y, CD38 IgG Z, CD38×CD3 IgG, and IgM B-2. FIG. 5C shows IFN-γ levels that resulted following treatment with increasing concentration of CD38 IgG Y, CD38 IgG Z, CD38×CD3 IgG, and IgM B-2.

These data show that CD38×CD3 IgG and IgM B-2 had similar levels of H929 cell killing, but IgM B-2 resulted in lower cytokine release than CD38×CD3 IgG.

TABLE 16
Functional Properties of CD38 × CD3 IgM on Co-Culture
of PBMCs and Luciferase-Tagged H929 Cells

| | EC₅₀ for | Maximum % | Maximum | Maximum |
|---|---|---|---|---|
| | Killing of | Killing of | TNF-α | IFN-γ |
| | H929 Cells | H929 | levels | levels |
| Antibody | (pM) | cells | (pg/mL) | (pg/mL) |
| IgM B-2 | 24 | 87 | 275 | 620 |
| CD38 IgG Y | 254 | 30 | <29 | 56 |
| CD38 IgG Z | 14 | 26 | <20 | <20 |
| CD38 × CD3 IgG | 25 | 94 | 606 | 1440 |

### Example 11: CD38×CD3 IgM Induces Greater CD8+ T Cell Proliferation and Reduces Highly Immunosuppressive CD38hi Regulatory T Cells

The effect of the CD38×CD3 IgM, IgM B-2, on CD8+ T cell and CD38hi regulatory T cell proliferation was tested as explained in Example 4. A co-culture of H929 and PBMC from three different donors were used, along with an extended incubation for 7 days with 5 nM CD38×CD3 IgG or IgM B-2 treatment.

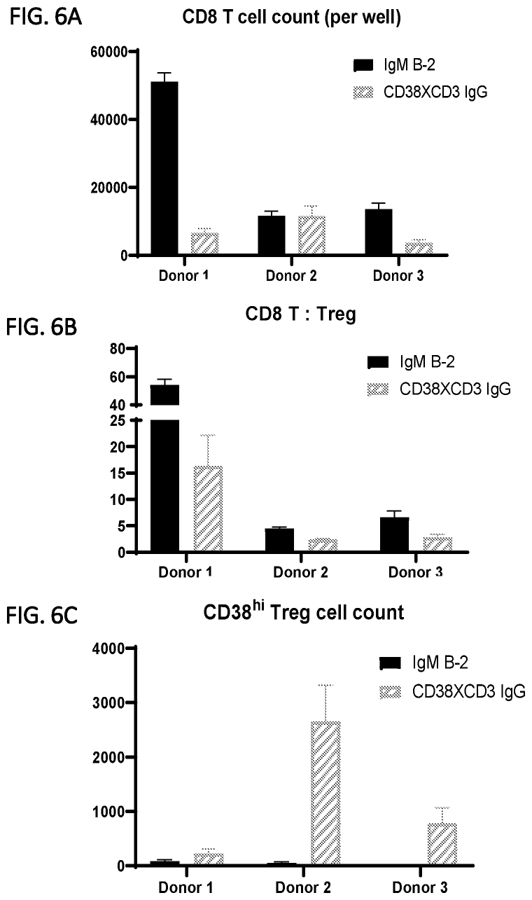

FIG. 6A shows CD8+ T cell count for each PBMC donor, following CD38×CD3 IgG or IgM B-2 treatment. FIG. 6B shows the ratio of CD8+ T cell count to regulatory T cell count (Treg) for each PBMC donor, following CD38×CD3 IgG or IgM B-2 treatment. FIG. 6C shows CD38hi regulatory T cell count for each PBMC donor, following CD38×CD3 IgG or IgM B-2 treatment.

These data show that IgM B-2 induced greater CD8+ T cell proliferation than CD38×CD3 IgG and reduced highly immunosuppressive CD38hi regulatory T cells more than CD38×CD3 IgG.

### Example 12: CD38×CD3 IgM Inhibits In Vitro Human Multiple Myeloma Colony Formation

To test the effect of the CD38×CD3 IgM, IgM B-2, on the growth of multiple myeloma in vitro, a colony formation assay was used. Frozen bone marrow mononuclear cells from four distinct multiple myeloma (MM) donors and two normal marrow donors were thawed and resuspended at 0.5×10⁶ cells/mL in a liquid-based medium containing 10% PHA conditioned medium (MM marrow) or cytokines (normal marrow) and plated into wells of a 24 well plate containing the test antibodies or solvent controls. The cells were incubated in liquid culture for 120 hours. Following the incubation, the cells within each well were dispersed carefully by pipetting. Four hundred microliters of cells (and medium) were removed from each well and added to 4.0 mL of methylcellulose containing 10% PHA conditioned medium (MM patients) or cytokines (normal donors). The tubes of methylcellulose were vortexed to ensure equal distribution of cells throughout the matrix. Triplicate cultures in 35 mm dishes were set up for each condition. The replicate dishes were placed at 37° C., 5% CO₂ for a total of 14-16 days, after which the resultant colonies were evaluated and enumerated based on morphology.

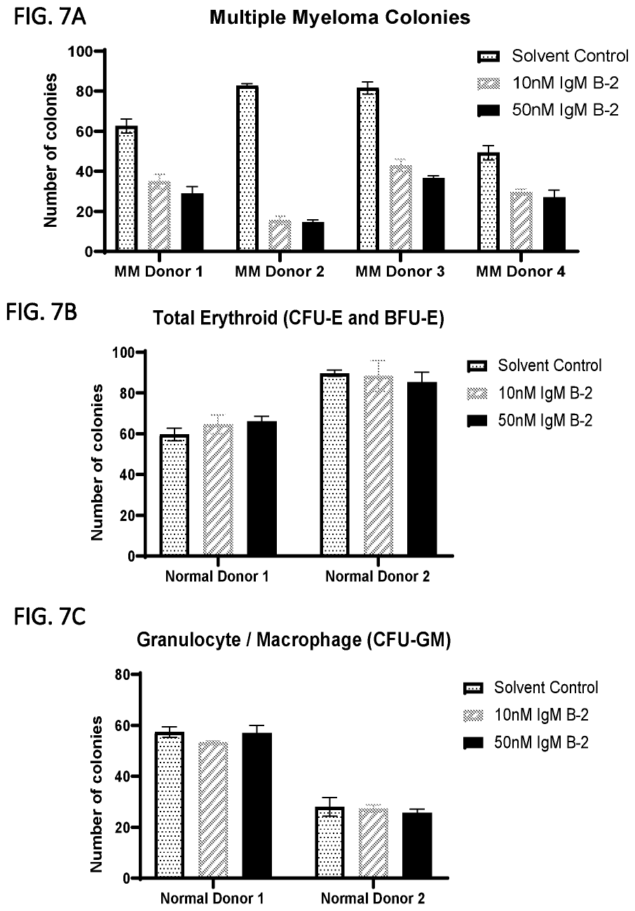

FIG. 7A shows in vitro colony formation of multiple myeloma cells from four different donors, following treatment with a solvent control, 10 nM IgM B-2, or 50 nM IgM B-2.

FIG. 7B shows in vitro colony formation of erythroid cells from two different normal bone marrow donors, following treatment with a solvent control, 10 nM IgM B-2, or 50 nM IgM B-2. Total erythroid cells were measured at the CFU-E (colony forming unit-erythroid) and BFU-E (erythroid burst-forming unit) stages of development.

FIG. 7C shows in vitro colony formation of granulocyte-macrophage progenitors (CFU-GM) from two different normal bone marrow donors, following treatment with a solvent control, 10 nM IgM B-2 or 50 nM IgM B-2.

These data show that IgM B-2 inhibited multiple myeloma colony formation but did not inhibit normal erythroid or granulocyte/macrophage colony formation.

### Example 12: CD38×CD3 IgM Exhibits Reduced Fratricide Compared to CD38×CD3 IgG In Vitro

Designing cancer immunotherapies remains a challenge because many targetable antigens, such as CD38, are shared by T cells and tumor cells. This shared expression of antigens can cause T cells to undergo self-killing or “fratricide” and reduce the effect of the immunotherapy on tumor cells. To investigate the effect of fratricide from treatment with the CD38×CD3 IgM, IgM B-2, the following experiments were performed.

T cell fratricide potential was tested in vitro using activated/expanded CD3+ pan T cells that have higher CD38 expression levels. Naïve T cells were activated and expanded using T cell activation/expansion kit from Miltenyi Biotec (Cat #130-091-441). Activated T cells were then treated in vitro with 5 nM CD38×CD3 IgG or IgM B-2. T cell fratricide was evaluated at 72h by flow cytometry analysis to quantitate live CD4+ and CD8+ T cells. Number of live cells per well was then normalized to the untreated control wells.

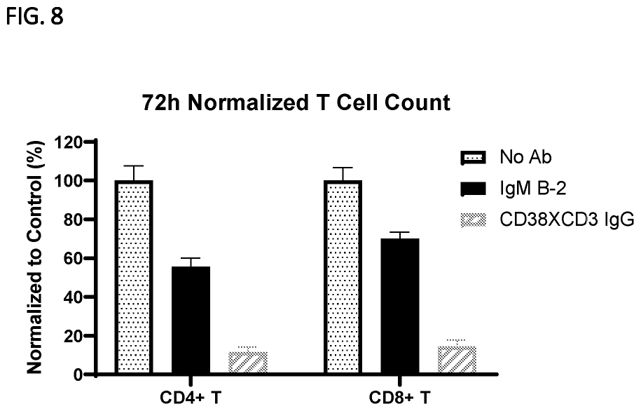

FIG. 8 shows IgM B-2 exhibits less T cell fratricide than CD38×CD3 IgG for both CD4+ and CD8+ T cells.

### Example 13: CD38×CD3 IgM Inhibits Disseminated Tumor Growth In Vivo in a Dose-Dependent Manner

Two million luciferase-tagged NCI-H929 (B lymphocytes isolated by malignant effusion from a 62-year-old female plasmacytoma multiple myeloma patient) cells were injected iv. to female NSG mice between 7-8 weeks old from the Jackson Laboratory. The day of tumor inoculation was considered as Do of the study. 20 million in vitro expanded human pan T cells were then injected iv. at 6 days post tumor implant. 24 hours post T cell implant, animals received vehicle, CD38×CD3 IgG or IgM B-2 treatment every 3 days at different dose levels. Tumor growth was monitored every week by bioluminescence imaging. Prior to imaging, the animals were injected intraperitoneally with 150 μg/kg of D-luciferin (Promega). Fifteen minutes later the animals were imaged using SonoVol STRATA imager and the imaging was done on D₆, D₁₂, D₂₀, D₂₆, and D₃₃.

Survival blood samples were taken at 5 days after 1^{st} dose of antibody treatment by retro-orbital bleed for flow analysis. Some animals were sacrificed to harvest bone marrow and spleen samples for flow analysis as well. T cells in the blood, bone marrow and spleen samples were identified with lineage markers (CD3, CD4, CD8) and the cell counts quantitated using spiked-in counting beads.

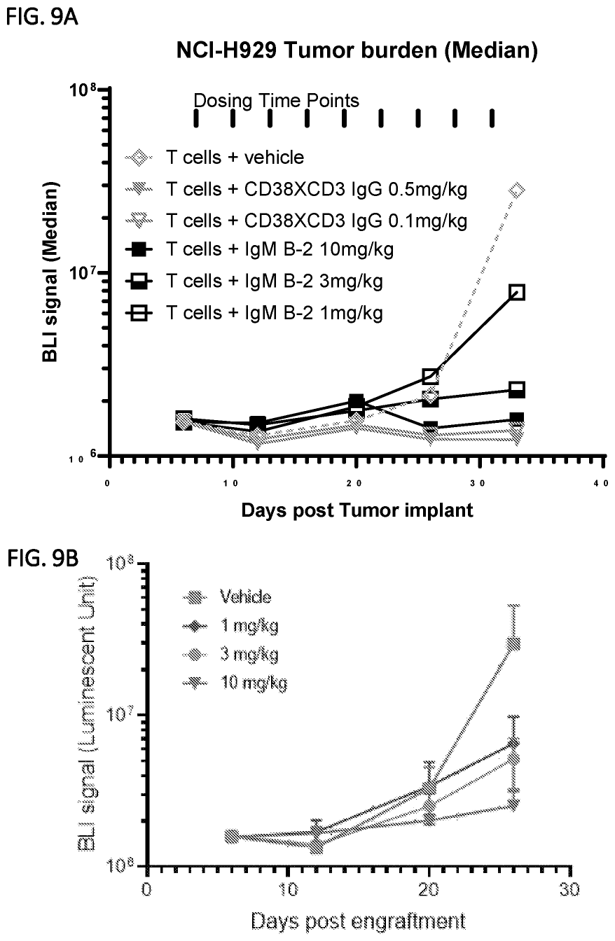

FIG. 9A shows the median BLI signal (tumor burden) over time in animals dosed with vehicle, CD38×CD3 IgG, or IgM B-2.

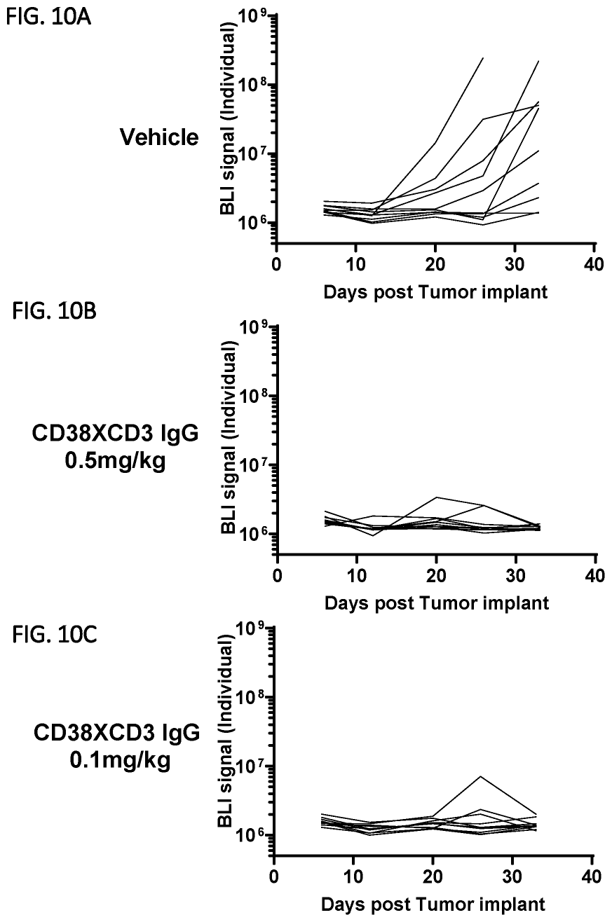

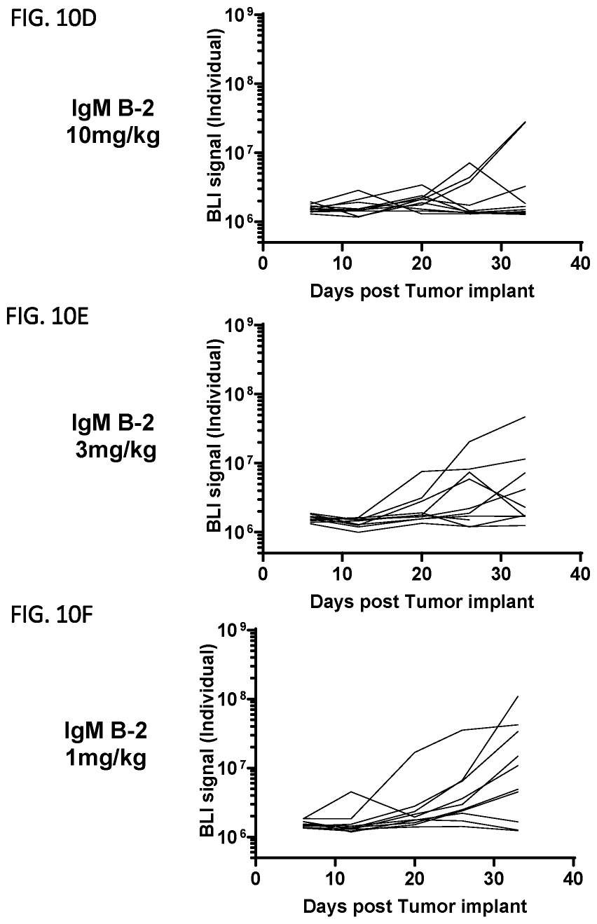

FIG. 10A-10F shows the BLI signal (tumor burden) over time for each of the animals treated with vehicle (FIG. 10A), 0.5 mg/kg CD38×CD3 IgG (FIG. 10B), 0.1 mg/kg CD38×CD3 IgG (FIG. 10C), 10 mg/kg IgM B-2 (FIG. 10D), 3 mg/kg IgM B-2 (FIG. 10E), or 1 mg/kg IgM B-2 (FIG. 10F).

These data show that IgM B-2 treatment resulted in a dose-dependent tumor inhibition effect.

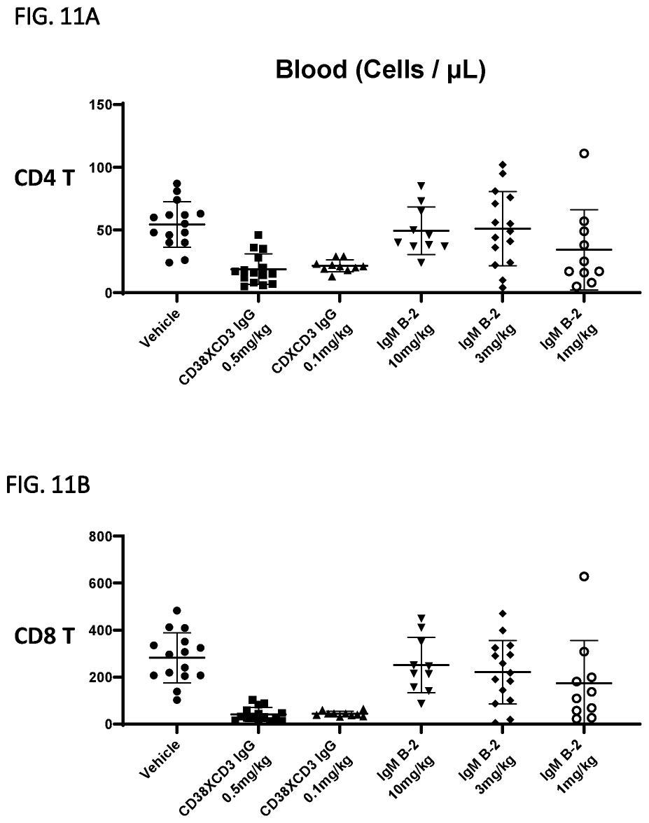

FIG. 11A-11B shows the number of CD4+ T cells (FIG. 11A) and CD8+ T cells (FIG. 11B) per microliter of blood in mice treated with vehicle, 0.5 or 0.1 mg/kg CD38×CD3 IgG, or 10, 3, or 1 mg/kg IgM B-2.

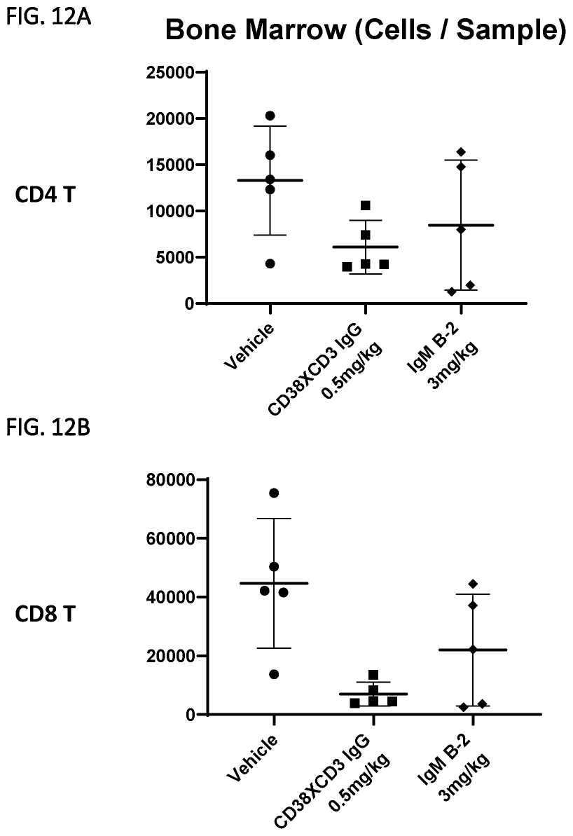

FIG. 12A-12B shows the number of CD4+ T cells (FIG. 12A) and CD8+ T cells (FIG. 12B) per bone marrow sample in mice treated with vehicle, 0.5 mg/kg CD38×CD3 IgG, or 3 mg/kg IgM B-2.

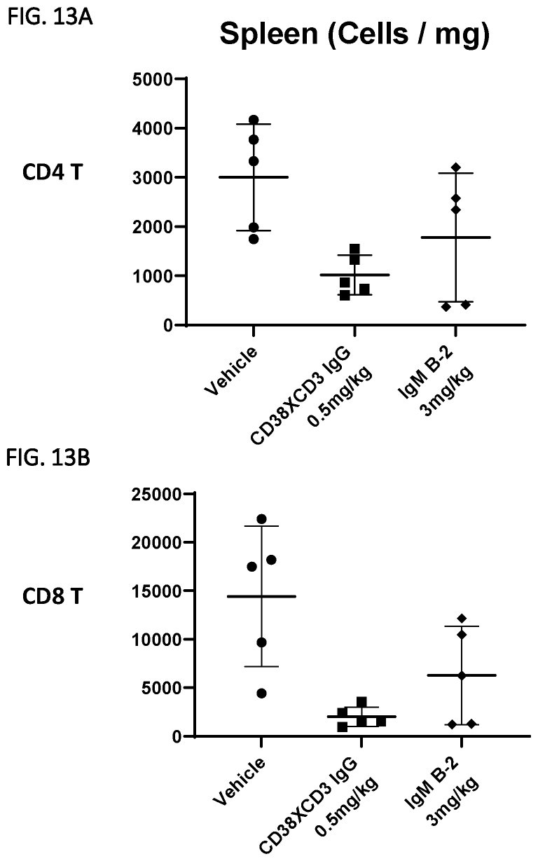

FIG. 13A-13B shows the number of CD4+ T cells (FIG. 13A) and CD8+ T cells (FIG. 13B) per mg of spleen sample in mice treated with vehicle, 0.5 mg/kg CD38×CD3 IgG, or 3 mg/kg IgM B-2.

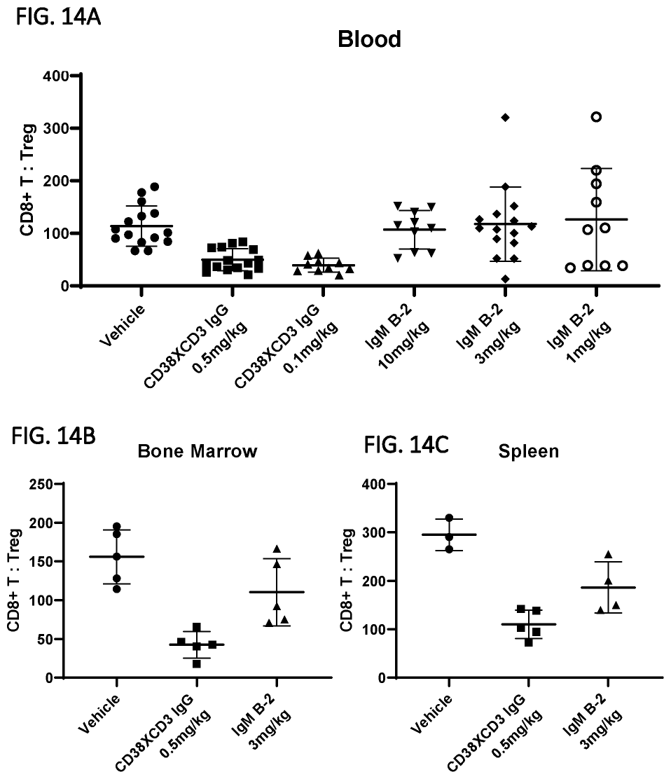

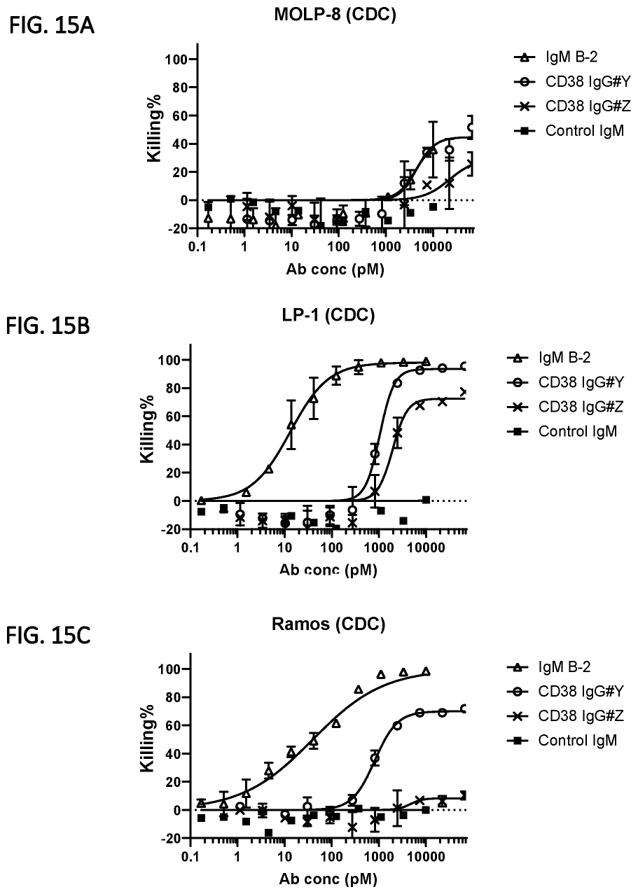

FIG. 14A shows the ratio of CD8+ T cells to regulatory T cells (Treg) (CD8+T: Treg) in peripheral blood of mice treated with vehicle, 0.1 or 0.5 mg/kg CD38×CD3 IgG or 1, 3, or 10 mg/kg IgM B-2.

FIG. 14B shows the ratio of CD8+ T cells to regulatory T cells (Treg) (CD8+T: Treg) in bone marrow of mice treated with vehicle, 0.5 mg/kg CD38×CD3 IgG or 3 mg/kg IgM B-2.

FIG. 14C shows the ratio of CD8+ T cells to regulatory T cells (Treg) (CD8+T: Treg) in spleen of mice treated with vehicle, 0.5 mg/kg CD38×CD3 IgG, or 3 mg/kg IgM B-2.

These data show that IgM B-2 treatment had reduced T cell fratricide compared to CD38×CD3 IgG, as IgM B-2-treated mice showed higher T cell counts in peripheral blood, bone marrow and spleen.

This study was repeated with four cohorts of ten mice each, but with only seven doses (administered on D₇, D₁₀, D₁₃, D₁₆, D₁₉, D₂₂, and D₂₅) of IGM B-2 instead of nine. The mice were light imaged on a Living Image 4.7.1 (PERKIN ELMER, Waltham, Massachusetts). Otherwise, the trial conditions were identical to those described above. FIG. 9B shows the tumor growth during the course of this study out to D₂₆, the last day on which all of the mice were still alive. Dose-dependent tumor regression was seen in the treatment groups, but none were statistically significant (p>0.009) compared to the vehicle control group.

### Example 14: Complement-Dependent Cytotoxicity Activity of CD38×CD3 IgM

Complement-dependent cytotoxicity (CDC) was measured in a plate-based assay. Briefly, various cell lines exhibiting various sensitivities to CDC, including MOLP-8 (multiple myeloma, DSMZ Cat #ACC 569), LP-1 (multiple myeloma, DSMZ Cat #ACC 41), Ramos (Burkitt's lymphoma, ATCC Cat #CRL-1596), Raji (Burkitt's lymphoma, ATCC Cat #CCL-86), RPMI-8226 (multiple myeloma, ATCC Cat #CCL-155), NCI-H929 (multiple myeloma, ATCC Cat #CRL-9068), OPM-2 (multiple myeloma, DSMZ Cat #ACC 50), and MOLM-13 (acute myeloid leukemia, DSMZ Cat #ACC 554), were washed and resuspended at a cell density of 1.0×10⁶ cells/mL in assay medium (RPMI 1640 medium with 10% heat inactivated FBS) and 10 μL/well was aliquoted to the wells of a 384 well plate (Thermo, catalog #164610). Purified antibodies IgM B-2, CD38 IgG #Y, and CD38 IgG #Z, and an IgM isotype control antibody were diluted to 90 or 30 μg/mL and then 3-fold serially diluted in medium and 10 μL/well was added to the cells in the 384 well plate. Normal human serum complement (NHS; Quidel, catalog #A113) was diluted to 30% in medium and 10 μL/well was added to the cell-antibody mixture in the 384 well plate. The plate was incubated for 4 h at 37° C. in a humidified 5% CO₂ incubator. Cell viability was determined by addition of 15 μL/well CellTiter-Glo reagent (Promega, catalog #G7572), which produces a luminescent signal in direct proportion to the amount of ATP produced by metabolically active cells. After gentle mixing on a plate shaker (2 min, 200 rpm) followed by incubation for 10 min at ambient temperature, luminescent data were collected on an En Vision plate reader (Perkin-Elmer) and analyzed with GraphPad Prism using a 4-parameter logistic model.

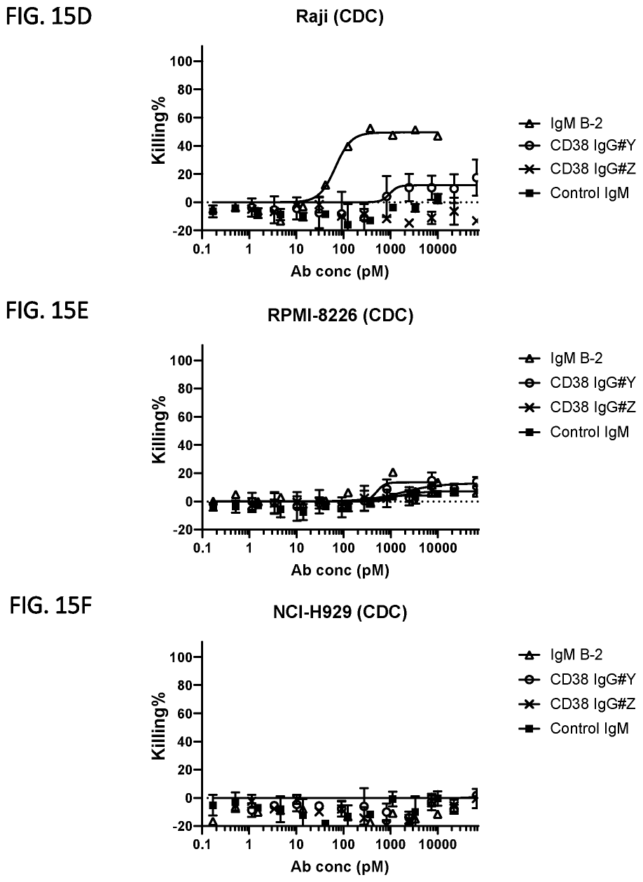

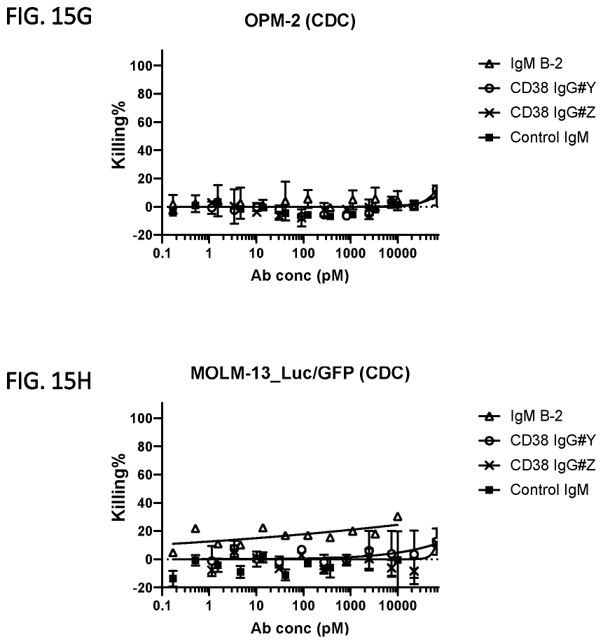

The results are shown in FIGS. 15A-15H. These data show that IgM B-2 exhibited superior CDC killing in MM cell lines that are sensitive to CDC.

### Example 15: CD38×CD3 IgM Exhibits Reduced Immune Effector Cell Killing Compared to CD38×CD3 IgG In Vitro

Primary NK cells (donor ID WO70522201466) and primary monocytes (donor ID WO70522201313) were treated in vitro with serial dilutions of CD38×CD3 IgG or IgM B-2. An irrelevant IgM (AB852) was included as a negative control. Immune effector cell viability was evaluated at 72h by flow cytometry analysis to quantitate live immune effector cells. Number of live cells per well was then normalized to untreated control wells.

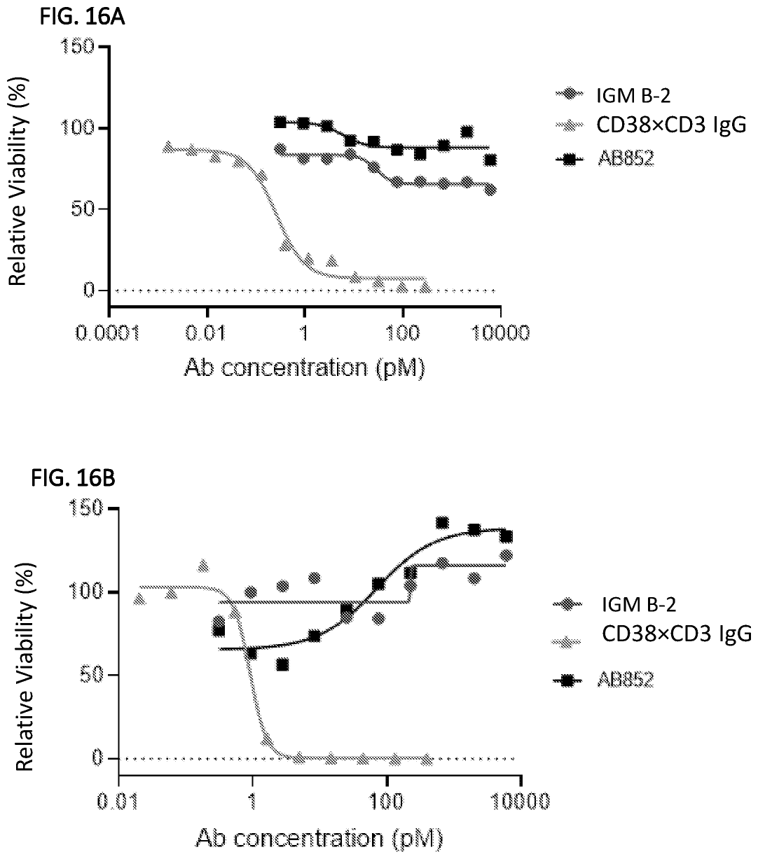

FIG. 16A shows that IgM B-2 kills fewer NK cells than does CD38×CD3 IgG. FIG. 16B shows that IgM B-2 kills fewer monocytes than does CD38×CD3 IgG.

### Example 15: Anti-Tumor Effect of IgM B-2 in a Disseminated NCI-H929-Luc-Gfp-Puro Tumor Model in NSG-MHC I/II DKO Mice Humanized with PBMCs

The anti-tumor effect of IgM B-2 was evaluated in a humanized CD38-positive NCI-H929-luc-gfp-Puro model using NSG-MHC I/II DKO mice. NSG-MHC I/II DKO mice are deficient in MHC class I and class II expression. When engrafted with human donor PBMCs, these mice have delayed and mild onset of graft vs. host disease (GVHD) and a longer period of induction of effector phase of T cell responses in comparison to regular NSG mice.

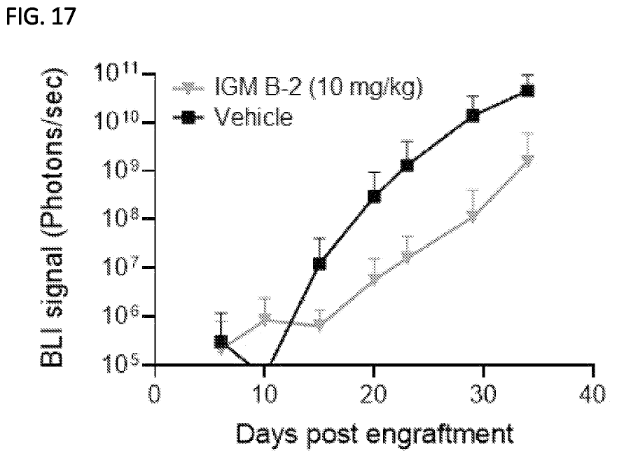

The day of tumor inoculation was considered as Do of the study. On D₋₁₄, each mouse was engrafted intravenously with 10 million healthy human PBMCs (purchased from IQ BIOSCIENCES). On D₃, cohorts of 8 mice each were treated with either vehicle or IgM B-2, administered intravenously for 11 doses, at 10 mg/kg with three times a week dosing of the antibody (FIG. 17) for 15 weeks. The control cohort was dosed with vehicle alone for only 10 weeks. The animals on the study reached tumor endpoint of 2000 mm³. Prior to imaging, the animals were injected intraperitoneally with 150 μg/kg of D-luciferin (Promega). Fifteen minutes later the animals were imaged using SonoVol STRATA imager and the imaging was done on D₆, D₁₀, D₁₅, D₂₀, D₂₄, D₂₉, and D₃₄.

FIG. 17 shows the effect of treatment with IGM B-2 in this disseminated NCI-H929-luc-gfp-Puro tumor model in NSG-MHC I/II DKO mice humanized with PBMCs. Error bars represent mean±SEM of 9 animals per group. Statistical analyses (Kruskal-Wallis tests with Dunn's test for multiple comparisons) represent comparisons to vehicle control group. Each vertical line indicates a day of antibody dosing. Tumor regression in the 10 mg/kg treatment group was not statistically significant (p=0.45) in comparison to the vehicle group.

### Example 16: Anti-Tumor Effect of IgM B-2 in a Multiple Myeloma Model in NSG-MHC I/II DKO Mice Humanized with PBMCs

The anti-tumor effect of IGM B-2 was evaluated in a CD38-positive subcutaneous multiple myeloma xenograft model in PBMC humanized NSG-MHCI/II DKO mice. Five million NCI-H929-gfp-luc-puro cells were implanted subcutaneously on the right flank of NSG-MHC I/II DKO mice. The cells were implanted at a volume of 100 μL/mouse in PBS. The day of tumor inoculation was considered as Do of the study. On D-14, each mouse was engrafted intravenously with 10 million healthy human PBMCs (purchased from IQ BIOSCIENCES). The animals were dosed three times per week with either vehicle or IGM B-2 administered intravenously for 15 doses at 1, 3, 10, and 30 mg/kg.

Tumors were measured using calipers and the tumor volume calculated using the formula, TV=(L×W²)/2, where L is the length of the tumor (the longest tumor dimension), and W is the width of the tumor. Body weight (BW) and tumors were measured twice weekly and the animals on study were monitored daily for signs of morbidity and mortality. Dosing, blood collection, tumor, and BW measurements were conducted in a laminar flow cabinet. Individual animals were euthanized when they reached the study endpoint-tumor volume of 2000 mm³ and body condition score (BCS) score of 2 or lower. Animals experiencing body weight loss (BWL) of >10% were administered lactated ringers solution (LRS), provided with hydrogel cups and softened food pellets. They were monitored daily and euthanized if the BWL was >20% relative to their BW at the start of the study (the first day of tumor measurement).

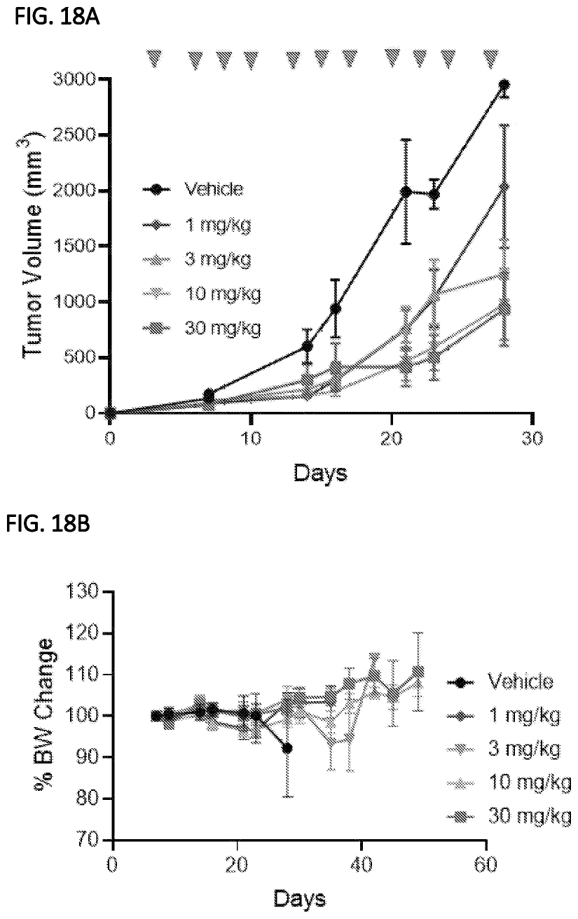

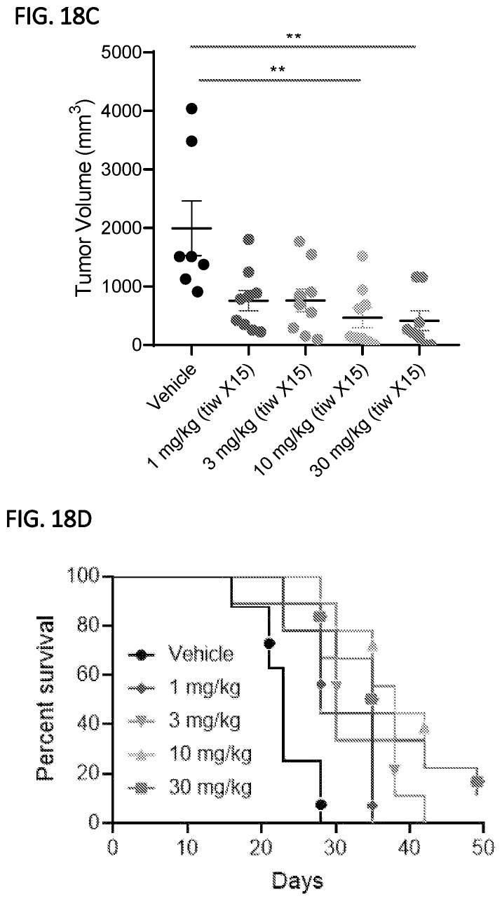

Dose-dependent tumor regression was seen in all the treatment groups (FIG. 18A). None of the animals exhibited any body weight loss (FIG. 18B). The animals in the vehicle group reached tumor endpoint of 2000 mm³ by D₂₈, but the animals in the treatment groups were monitored for survival and the study was terminated on D₄₉. At study termination, there was one tumor free mouse in each of the 30 mg/kg and 10 mg/kg groups. IGM B-2 treatment at 30 mg/kg and 10 mg/kg extended the median overall survival by 12 days and 13.5 days respectively, when compared to the vehicle treated group (FIG. 18C).

Comparison of tumor volumes on D₂₁ of the study, yielded a statistically significant p value for the 30 mg/kg group (p=0.0049) and 10 mg/kg group (p=0.0045) in comparison to the vehicle group (FIG. 18D). Percent tumor growth inhibition (% TGI) was calculated on D₂₁. The 30 mg/kg group exhibited the largest % TGI of 81.9%. In comparison 10, 3, and 1 mg/kg exhibited a % TGI of 80%, 63.8%, and 63.6%, respectively.

### Example 17: Anti-Tumor Effect of IgM B-2 in Humanized CD38-Positive Raji Xenograft Tumor Model

The Raji line of lymphoblast-like cells was established from a Burkitt's lymphoma. Three million Raji cells were implanted subcutaneously on the right flank of each of 5 cohorts of NSG-MHC I/II DKO mice (9 mice/cohort). The cells were implanted at a volume of 100 μL/mouse in 1×PBS. The day of tumor inoculation was considered as Do of the study.

On D₋₁₄, each mouse was engrafted intravenously with 10 million healthy human PBMCs (purchased from IQ BIOSCIENCES). On D₃, the animals were dosed intravenously with either vehicle or IgM B-2 at 30, 10, 3, and 1 mg/kg, three times a week for 15 doses. A control cohort was dosed with vehicle alone three times a week for only 10 weeks. The animals on the study reached tumor endpoint of 2000 mm³.

Tumors were measured using calipers and the tumor volume calculated using the formula, TV=(L×W2)/2. BW and tumors were measured twice weekly and the animals on study were monitored daily for signs of morbidity and mortality. Dosing, blood collection, tumor, and BW measurements were conducted in a laminar flow cabinet. Individual animals were euthanized when they reached the study endpoint: tumor volume of 2000 mm³ and BCS score of 2 or lower. Animals experiencing BWL of >10% were administered lactated LRS, provided with hydrogel cups and softened food pellets. They were monitored daily and euthanized if the BWL was >20% relative to their BW at the start of the study (the first day of tumor measurement).

Kruskal-Wallis tests with Dunn's test for multiple comparisons were used to compare tumor volume between treatment groups. The Kaplan-Meier method was used to evaluate survival curves and Log-rank (Mantel-Cox) tests were used to compare treatment groups. Statistical analyses were performed using GraphPad Prism 8 software. For all the statistical tests performed, a two-tailed value of P<0.05 was considered statistically significant.

Tumor growth inhibition was calculated as (1−(Tₙ−T₀)/(Cn−C₀))×100, where Tₙ=mean tumor volume of the treatment group on day n, T₀=mean tumor volume of the treatment group on day 7, Cₙ=mean tumor volume of the control group on day n, and C₀=mean tumor volume of the control group on day 7.

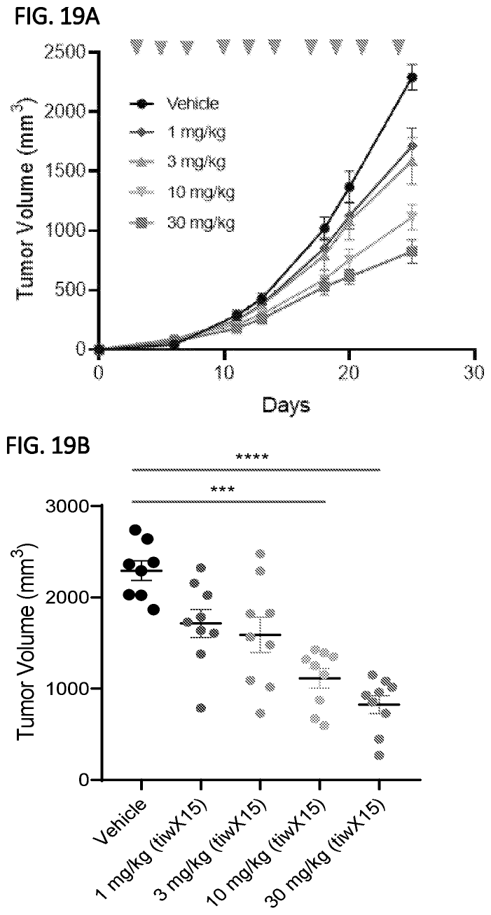

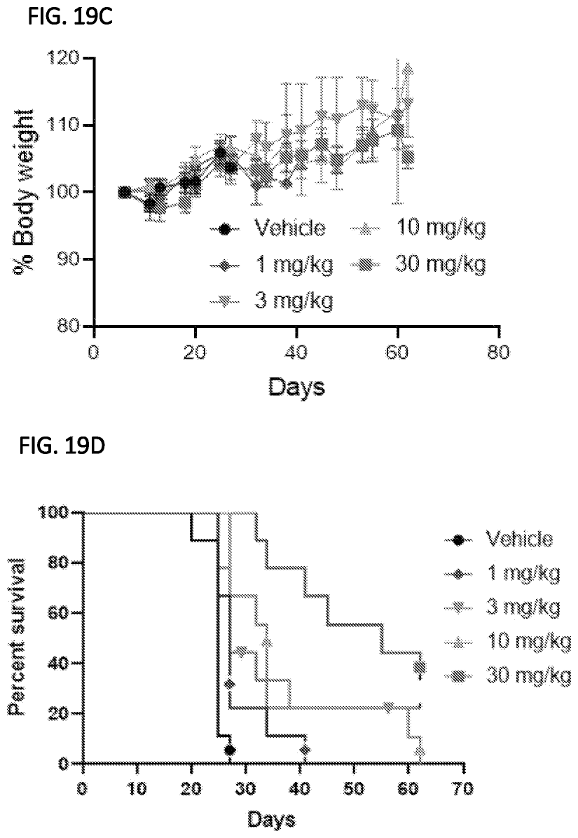

Dose-dependent tumor regression was seen in all the treatment groups (FIG. 19A). Comparison of tumor volumes on D₂₅ yielded a statistically significant p value for the 30 mg/kg group (p<0.0001) and 10 mg/kg group (p=0.0008) in comparison to the vehicle group (FIG. 19B). Percent tumor growth inhibition (% TGI) was calculated on D₂₅, when 8/9 mice in the vehicle group were alive. The highest dose group of 30 mg/kg group exhibited the largest % TGI of 66.4%. In comparison 10, 3 and 1 mg/kg exhibited a % TGI of 54.5%, 33.4% and 26.8%, respectively.

None of the animals exhibited any body weight loss (FIG. 19C). The animals in the vehicle group reached tumor endpoint of 2000 mm³ by D₂₇, but the animals in the treatment groups were monitored for survival and the study was terminated on D₆₃. At study termination, there was one tumor free mouse in the 30 mg/kg and two tumor free mice in the 3 mg/kg group. IGM B-2 treatment at 30 mg/kg extended the median overall survival by 18 days (FIG. 19D) in comparison to the vehicle treatment group.

TABLE 17
Sequences of exemplary CD3 binders
US5834597A

| SEQ ID | VH | | | | |
|---|---|---|---|---|---|
| 16 | QVQLVQSGAEVKKPGASVKVSCKASGYTFISYTMHWVRQAPGQGLEWMGYINPRSGYTHYNQKLKDKATLTADKSA | | | | |
| | STAYMELSSLRSEDTAVYYCARSAYYDYDGFAYWGQGTLVTVSS | | | | |
| SEQ ID | HCDR1 Sequence | SEQ ID | HCDR2 Sequence | SEQ ID | HCDR3 Sequence |
| 17 | SYTMH | 18 | YINPRSGYTHYNQKLKD | 19 | SAYYDYDGFAY |
| SEQ ID | VL | | | | |
| 20 | DIQMTQSPSSLSASVGDRVTITCSASSSVSYMNWYQQKPGKAPKRLIYDTSKLASGVPSRFSGSGSGTDETLTISS | | | | |
| | LQPEDFATYYCQQWSSNPPTFGGGTKVEIK | | | | |
| SEQ ID | LCDR1 Sequence | SEQ ID | LCDR2 Sequence | SEQ ID | LCDR3 Sequence |
| 21 | SASSSVSYMN | 22 | DTSKLAS | 23 | QQWSSNPPT |

WO2018208864

| SEQ ID | VH | | | | |
|---|---|---|---|---|---|
| 24 | EVQLLESGGGLVQPGGSLRLSCAASGETEDTYAMNWVRQAPGKGLEWVARIRSKYNNYATYYADSVKDRFTISRDD | | | | |
| | SKSTLYLOMESLRAEDTAVYYCVRHANFGAGYVSWFAHWGQGTLVTVSS | | | | |
| SEQ ID | HCDR1 Sequence | SEQ ID | HCDR2 Sequence | SEQ ID | HCDR3 Sequence |
| 25 | TYAMN | 26 | RIRSKYNNYATYYADSVKD | 27 | HANFGAGYVSWFAH |
| SEQ ID | VL | | | | |
| 28 | QTVVTQEPSLSVSPGGTVTLTCGSSTGAVTTSNYANWVQQTPGQAPRGLIGGTDKRAPGVPDRESGSLLGDKAALT | | | | |
| | ITGAQAEDEADYYCALWYSNHWVEGGGTKLTVL | | | | |
| SEQ ID | LCDR1 Sequence | SEQ ID | LCDR2 Sequence | SEQ ID | LCDR3 Sequence |
| 29 | GSSTGAVTTSNYAN | 30 | GTDKRAP | 31 | ALWYSNHWV |

WO2018208864

| SEQ ID | VH | | | | |
|---|---|---|---|---|---|
| 24 | EVQLLESGGGLVQPGGSLRLSCAASGETEDTYAMNWVRQAPGKGLEWVARIRSKYNNYATYYADSVKDRFTISRDD | | | | |
| | SKSTLYLQMESLRAEDTAVYYCVRHANFGAGYVSWFAHWGQGTLVTVSS | | | | |
| SEQ ID | HCDR1 Sequence | SEQ ID | HCDR2 Sequence | SEQ ID | HCDR3 Sequence |
| 25 | TYAMN | 26 | RIRSKYNNYATYYADSVKD | 27 | HANFGAGYVSWFAH |
| SEQ ID | VL | | | | |
| 32 | QTVVTQEPSLSVSPGGTVTLTCGSSTGAVTTSNYANWVQQTPGQAPRGLIGGTDKRAPGVPDRESGSLLGDKAALT | | | | |
| | ITGAQAEDEADYYCALWYSDLWVEGGGTKLTVL | | | | |
| SEQ ID | LCDR1 Sequence | SEQ ID | LCDR2 Sequence | SEQ ID | LCDR3 Sequence |
| 29 | GSSTGAVTTSNYAN | 30 | GTDKRAP | 33 | ALWYSDLWV |

WO2018208864

| SEQ ID | VH | | | | |
|---|---|---|---|---|---|
| 34 | QVQLVQSGAEVKKPGASVKVSCKASGENIKDYYMHWVRQAPGQRLEWMGWIDLENANTIYDAKFQGRVTITRDTSA | | | | |
| | STAYMELSSLRSEDTAVYYCARDAYGRYFYDVWGQGTLVTVSS | | | | |
| SEQ ID | HCDR1 Sequence | SEQ ID | HCDR2 Sequence | SEQ ID | HCDR3 Sequence |
| 35 | DYYMH | 36 | WIDLENANTIYDAKFQG | 37 | DAYGRYFYDV |
| SEQ ID | VL | | | | |
| 38 | DIVMTQSPDSLAVSLGERATINCKSSQSLLNARTGKNYLAWYQQKPGQPPKLLIYWASTRESGVPDRFSGSGSGTD | | | | |
| | FTLTISSLQAEDVAVYYCKQSYSRRTFGGGTKVEIK | | | | |
| SEQ ID | LCDR1 Sequence | SEQ ID | LCDR2 Sequence | SEQ ID | LCDR3 Sequence |
| 39 | KSSQSLINARTGKNY | 40 | WASTRES | 41 | KQSYSRRT |
| | LA | | | | |

WO2018208864

| SEQ ID | HCDR1 Sequence | SEQ ID | HCDR2 Sequence | SEQ ID | HCDR3 Sequence |
|---|---|---|---|---|---|
| 35 | DYYMH | 43 | WIDLENANTVYDAKFQG | 37 | DAYGRYFYDV |
| SEQ ID | VL | | | | |
| 44 | DIVMIQSPDSLAVSLGERATINCKSSQSLLNARTGKNYLAWYQQKPGQPPKLLIYWASTRESGVPDRFSGSGSGTD | | | | |
| | FTLTISSLQAEDVAVYYCKQSYFRRTFGGGTKVEIK | | | | |
| SEQ ID | LCDR1 Sequence | SEQ ID | LCDR2 Sequence | SEQ ID | LCDR3 Sequence |
| 39 | KSSQSLINARTGKNYLA | 40 | WASTRES | 45 | KQSYFRRT |

WO2018208864

| SEQ ID | VH | | | | |
|---|---|---|---|---|---|
| 46 | QVQLVQSGAEVKKPGASVKVSCKASGFNIKDYYMHWVRQAPGQRLEWIGWIDLENANTVYDAKFQGRVTITRDTSA | | | | |
| | STAYMELSSLRSEDTAVYYCARDAYGQYFYDVWGQGTLVTVSS | | | | |
| SEQ ID | HCDR1 Sequence | SEQ ID | HCDR2 Sequence | SEQ ID | HCDR3 Sequence |
| 35 | DYYMH | 43 | WIDLENANTVYDAKFQG | 47 | DAYGQYFYDV |
| SEQ ID | VL | | | | |
| 48 | DIVMIQSPDSLAVSLGERATINCKSSQSLLNARTGKNYLAWYQQKPGQPPKLLIYWASTRESGVPDRFSGSGSGTD | | | | |
| | FTLTISSLQAEDVAVYYCTQSYFRRTFGGGTKVEIK | | | | |
| SEQ ID | LCDR1 Sequence | SEQ ID | LCDR2 Sequence | SEQ ID | LCDR3 Sequence |
| 39 | KSSQSLINARTGKNY | 40 | WASTRES | 49 | TQSYFRRT |
| | LA | | | | |

WO2015095392

| SEQ ID | VH | | | | |
|---|---|---|---|---|---|
| 50 | EVQLVESGGGLVQPKGSLKLSCAASGFTENTYAMNWVRQAPGKGLEWVARIRSKYNNYATYYADSVKDRFTISRDD | | | | |
| | SQSILYLQMNNLKTEDTAMYYCVRHGNFGNSYVSWFAYWGQGTLVTVSS | | | | |
| SEQ ID | HCDR1 Sequence | SEQ ID | HCDR2 Sequence | SEQ ID | HCDR3 Sequence |
| 25 | TYAMN | 26 | RIRSKYNNYATYYADSVKD | 51 | HGNFGNSYVSWFAY |
| | VL | | | | |
| SEQ ID | QAVVTQESALTTSPGETVTLTCRSSTGAVTTSNYANWVQEKPDHLFTGLIGGINKRAPGVPARFSGSLIGDKAALT | | | | |
| 52 | ITGAQTEDEATYFCALWYSNLWVFGGGTKLTVL | | | | |
| SEQ ID | LCDR1 Sequence | SEQ ID | LCDR2 Sequence | SEQ ID | LCDR3 Sequence |
| 53 | RSSTGAVTTSNYAN | 54 | GTNKRAP | 55 | ALWYSNLWV |

TABLE 18
Other sequences in disclosure

| SEQ | Nickname | |
|---|---|---|
| ID | (source) | Sequence |
| 1 | Human IgM | GSASAPTLFPLVSCENSPSDTSSVAVGCLAQDFLPDSITFSWKYKNNSDISSTR |
| | Constant | GFPSVLRGGKYAATSQVLLPSKDVMQGTDEHVVCKVQHPNGNKEKNVPLPVIAE |
| | region IMGT | LPPKVSVFVPPRDGFFGNPRKSKLICQATGFSPRQIQVSWLREGKQVGSGVTTD |
| | allele IGHM*03 | QVQAEAKESGPTTYKVTSTLTIKESDWLSQSMFTCRVDHRGLTFQQNASSMCVP |
| | (GenBank: | DQDTAIRVFAIPPSFASIFLTKSTKLTCLVTDLTTYDSVTISWTRQNGEAVKTH |
| | pir\|S37768\|) | TNISESHPNATFSAVGEASICEDDWNSGERFTCTVTHTDLPSPLKQTISRPKGV |
| | | ALHRPDVYLLPPAREQLNLRESATITCLVTGFSPADVFVQWMQRGQPLSPEKYV |
| | | TSAPMPEPQAPGRYFAHSILTVSEEEWNTGETYTCVVAHEALPNRVTERTVDKS |
| | | TGKPTLYNVSLVMSDTAGTCY |
| 2 | Human IgM | GSASAPTLFPLVSCENSPSDTSSVAVGCLAQDFLPDSITFSWKYKNNSDISSTR |
| | Constant | GFPSVLRGGKYAATSQVLLPSKDVMQGTDEHVVCKVQHPNGNKEKNVPLPVIAE |
| | region IMGT | LPPKVSVFVPPRDGFFGNPRKSKLICQATGESPRQIQVSWLREGKQVGSGVTTD |
| | allele IGHM*04 | QVQAEAKESGPTTYKVTSTLTIKESDWLGQSMFTCRVDHRGLTFQQNASSMCVP |
| | (GenBank: | DQDTAIRVFAIPPSFASIFLTKSTKLTCLVTDLTTYDSVTISWTRQNGEAVKTH |
| | sp\|P01871.4\|) | TNISESHPNATFSAVGEASICEDDWNSGERFTCTVTHTDLPSPLKQTISRPKGV |
| | | ALHRPDVYLLPPAREQLNLRESATITCLVTGFSPADVFVQWMQRGQPLSPEKYV |
| | | TSAPMPEPQAPGRYFAHSILTVSEEEWNTGETYTCVVAHEALPNRVTERTVDKS |
| | | TGKPTLYNVSLVMSDTAGTCY |
| 3 | Human IgA1 | ASPTSPKVFPLSLCSTQPDGNVVIACLVQGFFPQEPLSVTWSESGQGVTARNFP |
| | heavy chain | PSQDASGDLYTTSSQLTLPATQCLAGKSVTCHVKHYTNPSQDVTVPCPVPSTPP |
| | constant | TPSPSTPPTPSPSCCHPRLSLHRPALEDLLLGSEANLTCTLTGLRDASGVTFTW |
| | region, e.g., | TPSSGKSAVQGPPERDLCGCYSVSSVLPGCAEPWNHGKTFTCTAAYPESKTPLT |
| | amino acids | ATLSKSGNTFRPEVHLLPPPSEELALNELVTLTCLARGFSPKDVLVRWLQGSQE |
| | 144 to 496 of | LPREKYLTWASRQEPSQGTTTFAVTSILRVAAEDWKKGDTFSCMVGHEALPLAF |
| | GenBank | TQKTIDRLAGKPTHVNVSVVMAEVDGTCY |
| | AIC59035.1 | |
| 4 | Human IgA2 | ASPTSPKVFPLSLDSTPQDGNVVVACLVQGFFPQEPLSVTWSESGQNVTARNFP |
| | heavy chain | PSQDASGDLYTTSSQLTLPATQCPDGKSVTCHVKHYTNSSQDVTVPCRVPPPPP |
| | constant | CCHPRLSLHRPALEDLLLGSEANLTCTLTGLRDASGATFTWTPSSGKSAVQGPP |
| | region, e.g., | ERDLCGCYSVSSVLPGCAQPWNHGETFTCTAAHPELKTPLTANITKSGNTFRPE |
| | amino acids 1 | VHLLPPPSEELALNELVTLTCLARGESPKDVLVRWLQGSQELPREKYLTWASRQ |
| | to 340 of | EPSQGTTTYAVTSILRVAAEDWKKGETFSCMVGHEALPLAFTQKTIDRMAGKPT |
| | GenBank | HINVSVVMAEADGTCY |
| | P01877.4 | |
| 5 | Precursor | MLLFVLTCLLAVEPAISTKSPIFGPEEVNSVEGNSVSITCYYPPTSVNRHTRKY |
| | Human | WCRQGARGGCITLISSEGYVSSKYAGRANLTNFPENGTFVVNIAQLSQDDSGRY |
| | Secretory | KCGLGINSRGLSFDVSLEVSQGPGLLNDTKVYTVDLGRTVTINCPFKTENAQKR |
| | Component | KSLYKQIGLYPVLVIDSSGYVNPNYTGRIRLDIQGTGQLLFSVVINQLRLSDAG |
| | | QYLCQAGDDSNSNKKNADLQVLKPEPELVYEDLRGSVTFHCALGPEVANVAKFL |
| | | CRQSSGENCDVVVNTLGKRAPAFEGRILLNPQDKDGSFSVVITGLRKEDAGRYL |
| | | CGAHSDGQLQEGSPIQAWQLFVNEESTIPRSPTVVKGVAGGSVAVLCPYNRKES |
| | | KSIKYWCLWEGAQNGRCPLLVDSEGWVKAQYEGRLSLLEEPGNGTFTVILNQLT |
| | | SRDAGFYWCLTNGDTLWRTTVEIKIIEGEPNLKVPGNVTAVLGETLKVPCHFPC |
| | | KFSSYEKYWCKWNNTGCQALPSQDEGPSKAFVNCDENSRLVSLTLNLVTRADEG |
| | | WYWCGVKQGHFYGETAAVYVAVEERKAAGSRDVSLAKADAAPDEKVLDSGFREI |
| | | ENKAIQDPRLFAEEKAVADTRDQADGSRASVDSGSSEEQGGSSRALVSTLVPLG |
| | | LVLAVGAVAVGVARARHRKNVDRVSIRSYRTDISMSDFENSREFGANDNMGASS |
| | | ITQETSLGGKEEFVATTESTTETKEPKKAKRSSKEEAEMAYKDFLLQSSTVAAE |
| | | AQDGPQEA |
| 6 | Precursor | MKNHLLFWGVLAVFIKAVHVKAQEDERIVLVDNKCKCARITSRIIRSSEDPNED |
| | Human J Chain | IVERNIRIIVPLNNRENISDPTSPLRTRFVYHLSDLCKKCDPTEVELDNQIVTA |
| | | TQSNICDEDSATETCYTYDRNKCYTAVVPLVYGGETKMVETALTPDACYPD |
| 7 | Mature Human J | QEDERIVLVDNKCKCARITSRIIRSSEDPNEDIVERNIRIIVPLNNRENISDPT |
| | Chain | SPLRTRFVYHLSDLCKKCDPTEVELDNQIVTATQSNICDEDSATETCYTYDRNK |
| | | CYTAVVPLVYGGETKMVETALTPDACYPD |
| 8 | J Chain Y102A | QEDERIVLVDNKCKCARITSRIIRSSEDPNEDIVERNIRIIVPLNNRENISDPT |
| | mutation | SPLRTRFVYHLSDLCKKCDPTEVELDNQIVTATQSNICDEDSATETCATYDRNK |
| | | CYTAVVPLVYGGETKMVETALTPDACYPD |
| 9 | “5” Peptide | GGGGS |
| | linker | |
| 10 | “10” Peptide | GGGGSGGGGS |
| | linker | |
| 11 | “15” Peptide | GGGGSGGGGSGGGGS |
| | linker | |
| 12 | “20” Peptide | GGGGSGGGGSGGGGSGGGGS |
| | linker | |
| 13 | “25” Peptide | GGGGSGGGGSGGGGSGGGGSGGGGS |
| | Linker | |
| 14 | human CD38 | MANCEFSPVSGDKPCCRLSRRAQLCLGVSILVLILVVVLAVVVPRWRQQWSGPG |
| | (GenBank: | TTKRFPETVLARCVKYTEIHPEMRHVDCQSVWDAFKGAFISKHPCNITEEDYQP |
| | BAA18966.1) | LMKLGTQTVPCNKILLWSRIKDLAHQFTQVQRDMFTLEDTLLGYLADDLTWCGE |
| | | FNTSKINYQSCPDWRKDCSNNPVSVFWKTVSRRFAEAACDVVHVMLNGSRSKIF |
| | | DKNSTFGSVEVHNLQPEKVQTLEAWVIHGGREDSRDLCQDPTIKELESIISKRN |
| | | IQFSCKNIYRPDKFLQCVKNPEDSSCTSEI |
| 15 | cyno CD38 | MANCEFSPVSGDKPCCRLSRRAQVCLGVCLLVLLILVVVVAVVLPRWRQQWSGS |
| | (GenBank: | GTTSRFPETVLARCVKYTEVHPEMRHVDCQSVWDAFKGAFISKYPCNITEEDYQ |
| | AAT36330.1) | PLVKLGTQTVPCNKTLLWSRIKDLAHQFTQVQRDMFTLEDMLLGYLADDLTWCG |
| | | EFNTFEINYQSCPDWRKDCSNNPVSVFWKTVSRRFAETACGVVHVMLNGSRSKI |
| | | FDKNSTFGSVEVHNLQPEKVQALEAWVIHGGREDSRDLCQDPTIKELESIISKR |
| | | NIRFFCKNIYRPDKFLQCVKNPEDSSCLSGI |
| 162 | OKT10 VH | EVKLQESGGGLVQPGGSLKLSCAASGFDFSRSWMNWVRQAPGKGLEWIGEINPD |
| | NCBI: | SSTINYTTSLKDKFIISRDNAKNTLYLQMTKVRSEDTALYYCARYGNWFPYWGQ |
| | ABA42888.1 | GTLVTVSA |
| 163 | OKT10 VL | DIVMTQSPKIMPTSVGDRVSVTCKASQNVDTNVAWYQQKPGQSPKALIYSASYR |
| | NCBI: | YSGVPDRFTGSGSGTDFTLTITNVQSEDLAEYFCQQYDSYPLTFGAGTKLDLK |
| | ABA42887.1 | |
| 164 | HB7 VH | TLVQLKQSGPSLVQPSQRLSITCTVSGFSLISYGVHWVRQSPGKGLEWLGVIWR |
| | PDB: 3RAJ | GGSTDYNAAFMSRLSITKDNSKSQVFFKMNSLQADDTAIYFCAKTLITTGYAMD |
| | | YWGQGTSVTVSS |
| 165 | HB7 VL | IQMTQSSSSFSVSLGDRVTITCKASEDIYNRLAWYQQKPGNAPRLLISGATSLE |
| | PDB: 3RAJ | TGVPSRFSGSGSGKDYTLSITSLQTEDVATYYCQQYWSTPTFGGGTKLEIK |
| 166 | SJ* | EVQLVESGGGLVQPKGSLKLSCAASGFTENTYAMNWVRQAPGKGLEWVARIRSK |
| | | YNNYATYYADSVKDRFTISRDDSQSILYLQMNNLKTEDTAMYYCVRHGNFGNSY |
| | | VSWFAYWGQGTLVTVSSGGGGSGGGGSGGGGSQAVVTQESALTTSPGETVTLTC |
| | | RSSTGAVTTSNYANWVQEKPDHLFTGLIGGTNKRAPGVPARFSGSLIGDKAALT |
| | | ITGAQTEDEAIYFCALWYSNLWVFGGGTKLTVLGGGGSGGGGSGGGGSQEDERI |
| | | VLVDNKCKCARITSRIIRSSEDPNEDIVERNIRIIVPLNNRENISDPTSPLRTR |
| | | FVYHLSDLCKKCDPTEVELDNQIVTATQSNICDEDSATETCATYDRNKCYTAVV |
| | | PLVYGGETKMVETALTPDACYPD |
| 167 | 1J* | EVQLLESGGGLVQPGGSLRLSCAASGFTFDTYAMNWVRQAPGKGLEWVARIRSK |
| | | YNNYATYYADSVKDRFTISRDDSKSTLYLQMESLRAEDTAVYYCVRHANFGAGY |
| | | VSWFAHWGQGTLVTVSSGGGGSGGGGSGGGGSQTVVTQEPSLSVSPGGTVTLTC |
| | | GSSTGAVTTSNYANWVQQTPGQAPRGLIGGTDKRAPGVPDRESGSLLGDKAALT |
| | | ITGAQAEDEADYYCALWYSNHWVFGGGTKLTVLGGGGSGGGGSGGGGSQEDERI |
| | | VLVDNKCKCARITSRIIRSSEDPNEDIVERNIRIIVPLNNRENISDPTSPLRTR |
| | | FVYHLSDLCKKCDPTEVELDNQIVTATQSNICDEDSATETCATYDRNKCYTAVV |
| | | PLVYGGETKMVETALTPDACYP |
| 168 | 2J* | EVQLLESGGGLVQPGGSLRLSCAASGFTFDTYAMNWVRQAPGKGLEWVARIRSK |
| | | YNNYATYYADSVKDRFTISRDDSKSTLYLQMESLRAEDTAVYYCVRHANFGAGY |
| | | VSWFAHWGQGTLVTVSSGGGGSGGGGSGGGGSQTVVTQEPSLSVSPGGTVTLTC |
| | | GSSTGAVTTSNYANWVQQTPGQAPRGLIGGTDKRAPGVPDRESGSLLGDKAALT |
| | | ITGAQAEDEADYYCALWYSDLWVFGGGTKLTVLGGGGSGGGGSGGGGSQEDERI |
| | | VLVDNKCKCARITSRIIRSSEDPNEDIVERNIRIIVPLNNRENISDPTSPLRTR |
| | | FVYHLSDLCKKCDPTEVELDNQIVTATQSNICDEDSATETCATYDRNKCYTAVV |
| | | PLVYGGETKMVETALTPDACYPD |
| 169 | 3J* | QVQLVQSGAEVKKPGASVKVSCKASGFNIKDYYMHWVRQAPGQRLEWIGWIDLE |
| | | NANTVYDAKFQGRVTITRDTSASTAYMELSSLRSEDTAVYYCARDAYGRYFYDV |
| | | WGQGTLVTVSSGGGGSGGGGSGGGGSDIVMTQSPDSLAVSLGERATINCKSSQS |
| | | LLNARTGKNYLAWYQQKPGQPPKLLIYWASTRESGVPDRESGSGSGTDFTLTIS |
| | | SLQAEDVAVYYCKQSYFRRTFGGGTKVEIKGGGGSGGGGSGGGGSQEDERIVLV |
| | | DNKCKCARITSRIIRSSEDPNEDIVERNIRIIVPLNNRENISDPTSPLRTRFVY |
| | | HLSDLCKKCDPTEVELDNQIVTATQSNICDEDSATETCATYDRNKCYTAVVPLV |
| | | YGGETKMVETALTPDACYPD |
| 170 | 4J* | QVQLVQSGAEVKKPGASVKVSCKASGFNIKDYYMHWVRQAPGQRLEWIGWIDLE |
| | | NANTVYDAKFQGRVTITRDTSASTAYMELSSLRSEDTAVYYCARDAYGQYFYDV |
| | | WGQGTLVTVSSGGGGSGGGGSGGGGSDIVMTQSPDSLAVSLGERATINCKSSQS |
| | | LLNARTGKNYLAWYQQKPGQPPKLLIYWASTRESGVPDRFSGSGSGTDFTLTIS |
| | | SLQAEDVAVYYCTQSYFRRTFGGGTKVEIKGGGGSGGGGSGGGGSQEDERIVLV |
| | | DNKCKCARITSRIIRSSEDPNEDIVERNIRIIVPLNNRENISDPTSPLRTRFVY |
| | | HLSDLCKKCDPTEVELDNQIVTATQSNICDEDSATETCATYDRNKCYTAVVPLV |
| | | YGGETKMVETALTPDACYPD |
| 171 | CD38 × CD3 IgG | EVQLVESGGGLVQPGGSLRLSCAASGFTESTYAMNWVRQAPGKGLEWVGRIRSK |
| | scFv-Fc chain | ANNYATYYADSVKGRFTISRDDSKNTLYLQMNSLRAEDTAVYYCVRHGNFGDSY |
| | US20180305465 | VSWFAYWGQGTLVTVSSGKPGSGKPGSGKPGSGKPGSQAVVTQEPSLTVSPGGT |
| | | VTLTCGSSTGAVTTSNYANWVQQKPGKSPRGLIGGTNKRAPGVPARFSGSLLGG |
| | | KAALTISGAQPEDEADYYCALWYSNHWVFGGGTKLTVLEPKSSDKTHTCPPCPA |
| | | PPVAGPSVFLFPPKPKDTLMISRTPEVTCVVVDVKHEDPEVKENWYVDGVEVHN |
| | | AKTKPREEQYNSTYRVVSVLTVLHQDWLNGKEYKCKVSNKALPAPIEKTISKAK |
| | | GQPREPQVYTLPPSREQMTKNQVKLTCLVKGFYPSDIAVEWESNGQPENNYKTT |
| | | PPVLDSDGSFFLYSKLTVDKSRWQQGNVFSCSVMHEALHNHYTQKSLSLSPGK |
| 172 | CD38 × CD3 IgG | DIVMTQSPSSLSASVGDRVTITCRASQNVDTWVAWYQQKPGQSPKALIYSASYR |
| | light chain | YSGVPDRFTGSGSGTDFTLTISSLQPEDFATYFCQQYDSYPLTFGGGTKLEIKR |
| | US20180305465 | TVAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSENRGEC |
| 173 | CD38 × CD3 IgG | EVQLVESGGGLVQPGGSLRLSCAASGFDFSRSWMNWVRQAPGKGLEWVSEINPD |
| | heavy chain | SSTINYATSVKGRFTISRDNSKNTLYLQMNSLRAEDTAVYYCARYGNWFPYWGQ |
| | US20180305465 | GTLVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSDTKVDKKVEP |
| | | KSCDKTHTCPPCPAPPVAGPSVFLFPPKPKDTLMISRTPEVTCVVVDVKHEDPE |
| | | VKFNWYVDGVEVHNAKTKPREEEYNSTYRVVSVLTVLHQDWLNGKEYKCKVSNK |
| | | ALPAPIEKTISKAKGQPREPQVYTLPPSREEMTKNQVSLTCDVSGFYPSDIAVE |
| | | WESDGQPENNYKTTPPVLDSDGSFFLYSKLTVDKSRWEQGDVFSCSVMHEALHN |
| | | HYTQKSLSLSPGK |
| 174 | CD38 IgG #Y | EVQLLESGGGLVQPGGSLRLSCAVSGFTENSFAMSWVRQAPGKGLEWVSAISGS |
| | Heavy Chain | GGGTYYADSVKGRFTISRDNSKNTLYLQMNSLRAEDTAVYFCAKDKILWFGEPV |
| | | FDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKV |
| | | DKRVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKGFY |
| | | PSDIAVEWESNGQPENNYKTTPPVLDSDGSFFLYSKLTVDKSRWQQGNVFSCSV |
| | | MHEALHNHYTQKSLSLSPGK |
| 175 | CD38 IgG #Y | EIVLTQSPATLSLSPGERATLSCRASQSVSSYLAWYQQKPGQAPRLLIYDASNR |
| | Light Chain US | ATGIPARFSGSGSGTDFTLTISSLEPEDFAVYYCQQRSNWPPTFGQGTKVEIKR |
| | 2020/0362049 | TVAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSENRGEC |
| 176 | CD38 IgG #Z | QVQLVQSGAEVAKPGTSVKLSCKASGYTFTDYWMQWVKQRPGQGLEWIGTIYPG |
| | Heavy Chain | DGDTGYAQKFQGKATLTADKSSKTVYMHLSSLASEDSAVYYCARGDYYGSNSLD |
| | US2021/0188996 | YWGQGTSVTVSSASTKGPSVFPLAPSSKSTSGGTAALGCLVKDYFPEPVTVSWN |
| | A1 | SGALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDVS |
| | | HEDPEVKFNWYVDGVEVHNAKTKPREEQYNSTYRVVSVLTVLHQDWLNGKEYKC |
| | | KVSNKALPAPIEKTISKAKGQPREPQVYTLPPSRDELTKNQVSLTCLVKGFYPS |
| | | DIAVEWESNGQPENNYKTTPPVLDSDGSFFLYSKLTVDKSRWQQGNVFSCSVMH |
| | | EALHNHYTQKSLSLSPGK |
| 177 | CD38 IgG #Z | DIVMTQSHLSMSTSLGDPVSITCKASQDVSTVVAWYQQKPGQSPRRLIYSASYR |
| | Light Chain | YIGVPDRFTGSGAGTDFTFTISSVQAEDLAVYYCQQHYSPPYTFGGGTKLEIKR |
| | US2021/0188996 | TVAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSENRGEC |

What is claimed is:

1. An antibody or antigen-binding fragment or derivative thereof comprising an antigen-binding domain that specifically binds to CD38, wherein the antigen-binding domain comprises a heavy chain variable region (VH) and light chain variable region (VL), wherein the VH and VL comprise six immunoglobulin complementarity determining regions HCDR1, HCDR2, HCDR3, LCDR1, LCDR2, and LCDR3,
wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 182, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95.

2. The antibody or antigen-binding fragment or derivative thereof of claim 1, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

3. The antibody or antigen-binding fragment or derivative thereof of claim 1 or claim 2, wherein the VH and VL comprise, respectively, an amino acid sequence at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142.

4. The antibody or antigen-binding fragment or derivative thereof of claim 3, wherein the VH and VL comprise, respectively, an amino acid sequence at least 80%, 85%, 90%, 95%, or 100% identical to the amino acid sequence of SEQ ID NO: 128 and SEQ ID NO: 133.

5. An antibody or antigen-binding fragment or derivative thereof comprising an antigen-binding domain that specifically binds to CD38, wherein the antigen-binding domain comprises a heavy chain variable region (VH) and light chain variable region (VL), wherein the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161.

6. The antibody or antigen-binding fragment or derivative thereof of any one of claims 1 to 5, which is a multimeric antibody comprising two, four, five, or six bivalent binding units and four, eight, ten, or twelve antigen-binding domains, wherein at least three, four, five, six, seven, eight, nine, ten, eleven, or twelve antigen-binding domains specifically bind to CD38;
wherein each binding unit comprises two heavy chains each comprising an IgA or IgM constant region or a multimerizing fragment or variant thereof, and wherein at least three, four, five, six, seven, eight, nine, ten, eleven, or twelve heavy chain constant regions or fragments or variants thereof is/are associated with a copy of the VH.

7. The antibody or antigen-binding fragment or derivative thereof of any one of claims 1 to 5, which is an Fv fragment, a single-chain Fv fragment (scFv), or a disulfide-linked Fv fragment (sdFv).

8. The antibody or antigen-binding fragment or derivative thereof any one of claims 1 to 5, comprising a complete antibody, an Fab fragment, an Fab′ fragment, or an F(ab′)2 fragment.

9. The antibody or antigen-binding fragment or derivative thereof of claim 8, which comprises a bivalent binding unit comprising two antigen-binding domains, wherein at least one antigen-binding domain specifically binds to CD38, wherein the binding unit comprises two heavy chains each comprising a heavy chain constant region or fragment or variant thereof, and wherein at least one heavy chain constant region or fragment or variant thereof is associated with a copy of the VH.

10. The antibody or antigen-binding fragment or derivative thereof of claim 9, wherein the heavy chains comprise IgG heavy chain constant regions or fragments or variants thereof fused to the C-terminus of the VH.

11. The antibody or antigen-binding fragment or derivative thereof of claim 10, wherein the binding unit further comprises two light chains each comprising a light chain constant region or fragment or variant thereof fused to the C-terminus of the VL.

12. A multimeric antibody comprising two, four, five, or six bivalent binding units, wherein each binding unit comprises two IgM or IgA heavy chain constant regions or multimerizing fragments or variants thereof, each associated with an antigen-binding domain, wherein at least three, four, five, six, seven, eight, nine, ten, eleven, or twelve of the antigen-binding domains are a CD38 antigen binding domain comprising a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71; SEQ ID NO: 57, SEQ ID NO: 58, SEQ ID NO: 59, SEQ ID NO: 61, SEQ ID NO: 62, and SEQ ID NO: 63; SEQ ID NO: 73, SEQ ID NO: 74, SEQ ID NO: 75, SEQ ID NO: 77, SEQ ID NO: 78, and SEQ ID NO: 79; SEQ ID NO: 81, SEQ ID NO: 82, SEQ ID NO: 83, SEQ ID NO: 85, SEQ ID NO: 86, and SEQ ID NO: 87; SEQ ID NO: 89, SEQ ID NO: 90, SEQ ID NO: 91, SEQ ID NO: 93, SEQ ID NO: 94, and SEQ ID NO: 95; SEQ ID NO: 97, SEQ ID NO: 98, SEQ ID NO: 99, SEQ ID NO: 101, SEQ ID NO: 102, and SEQ ID NO: 103; SEQ ID NO: 105, SEQ ID NO: 106, SEQ ID NO: 107, SEQ ID NO: 109, SEQ ID NO: 110, and SEQ ID NO: 111; SEQ ID NO: 113, SEQ ID NO: 114, SEQ ID NO: 115, SEQ ID NO: 117, SEQ ID NO: 118, and SEQ ID NO: 119; SEQ ID NO: 121, SEQ ID NO: 122, SEQ ID NO: 123, SEQ ID NO: 125, SEQ ID NO: 126, and SEQ ID NO: 127; SEQ ID NO: 65, SEQ ID NO: 134, SEQ ID NO: 67, SEQ ID NO: 69, SEQ ID NO: 70, and SEQ ID NO: 71; SEQ ID NO: 65, SEQ ID NO: 182, SEQ ID NO: 67, SEQ ID NO: 69, SEQ ID NO: 70, and SEQ ID NO: 71; SEQ ID NO: 65, SEQ ID NO: 135, SEQ ID NO: 67, SEQ ID NO: 69, SEQ ID NO: 70, and SEQ ID NO: 71; SEQ ID NO: 89, SEQ ID NO: 90, SEQ ID NO: 91, SEQ ID NO: 143, SEQ ID NO: 94, and SEQ ID NO: 95; SEQ ID NO: 89, SEQ ID NO: 90, SEQ ID NO: 91, SEQ ID NO: 144, SEQ ID NO: 94, and SEQ ID NO: 95; SEQ ID NO: 89, SEQ ID NO: 90, SEQ ID NO: 91, SEQ ID NO: 145, SEQ ID NO: 94, and SEQ ID NO: 95; or SEQ ID NO: 89, SEQ ID NO: 90, SEQ ID NO: 91, SEQ ID NO: 146, SEQ ID NO: 94, and SEQ ID NO: 95.

13. The multimeric antibody of claim 12, wherein the multimeric antibody is more potent than an equivalent amount of a bivalent IgG antibody comprising the VH and VL of the multimeric antibody.

14. The multimeric antibody of claim 13, which can direct complement-directed cytotoxicity (CDC) of a CD38-expressing cell at a higher potency than an equivalent amount of a bivalent IgG antibody comprising the VH and VL of the multimeric antibody.

15. The multimeric antibody of any one of claims 12 to 14, wherein the HCDR1, HCDR2, HCDR3, LCDR1, LCDR2, and LCDR3 comprise, respectively, the amino acid sequences of SEQ ID NO: 65, SEQ ID NO: 66, SEQ ID NO: 67, SEQ ID NO: 69, SEQ ID NO: 70, and SEQ ID NO: 71.

16. The multimeric antibody of any one of claims 12 to 14, wherein the VH and VL comprise, respectively, an amino acid sequence at least 80%, 85%, 90%, 95%, or 100% identical to the amino acid sequence of SEQ ID NO: 128 and SEQ ID NO: 133; SEQ ID NO: 56 and SEQ ID NO: 60; SEQ ID NO: 64 and SEQ ID NO: 68; SEQ ID NO: 72 and SEQ ID NO: 76; SEQ ID NO: 80 and SEQ ID NO: 84; SEQ ID NO: 88 and SEQ ID NO: 92; SEQ ID NO: 96 and SEQ ID NO: 100; SEQ ID NO: 104 and SEQ ID NO: 108; SEQ ID NO: 112 and SEQ ID NO: 116; SEQ ID NO: 120 and SEQ ID NO: 124; SEQ ID NO: 128 and SEQ ID NO: 68; SEQ ID NO: 129 and SEQ ID NO: 68; SEQ ID NO: 129 and SEQ ID NO: 133; SEQ ID NO: 130 and SEQ ID NO: 68; SEQ ID NO: 130 and SEQ ID NO: 133; SEQ ID NO: 131 and SEQ ID NO: 68; SEQ ID NO: 131 and SEQ ID NO: 133; SEQ ID NO: 132 and SEQ ID NO: 68; SEQ ID NO: 132 and SEQ ID NO: 133; SEQ ID NO: 64 and SEQ ID NO: 133; SEQ ID NO: 136 and SEQ ID NO: 86; SEQ ID NO: 136 and SEQ ID NO: 138; SEQ ID NO: 136 and SEQ ID NO: 139; SEQ ID NO: 136 and SEQ ID NO: 140; SEQ ID NO: 136 and SEQ ID NO: 141; SEQ ID NO: 136 and SEQ ID NO: 142; SEQ ID NO: 137 and SEQ ID NO: 86; SEQ ID NO: 137 and SEQ ID NO: 138; SEQ ID NO: 137 and SEQ ID NO: 139; SEQ ID NO: 137 and SEQ ID NO: 140; SEQ ID NO: 137 and SEQ ID NO: 141; SEQ ID NO: 137 and SEQ ID NO: 142; SEQ ID NO: 88 and SEQ ID NO: 138; SEQ ID NO: 88 and SEQ ID NO: 139; SEQ ID NO: 88 and SEQ ID NO: 140; SEQ ID NO: 88 and SEQ ID NO: 141; or SEQ ID NO: 88 and SEQ ID NO: 142.

17. The multimeric antibody of claim 16, wherein the VH and VL comprise, respectively, an amino acid sequence at least 80%, 85%, 90%, 95%, or 100% identical to the amino acid sequence of SEQ ID NO: 128 and SEQ ID NO: 133.

18. A multimeric antibody comprising two, four, five, or six bivalent binding units, wherein each binding unit comprises two IgM or IgA heavy chain constant regions or multimerizing fragments or variants thereof, each associated with an antigen-binding domain, wherein at least three, four, five, six, seven, eight, nine, ten, eleven, or twelve of the antigen-binding domains are a CD38 antigen binding domain comprising a heavy chain variable region (VH) and a light chain variable region (VL), wherein the VH and VL comprise, respectively, the amino acid sequence of SEQ ID NO: 147 and SEQ ID NO: 150; SEQ ID NO: 147 and SEQ ID NO: 151; SEQ ID NO: 147 and SEQ ID NO: 152; SEQ ID NO: 147 and SEQ ID NO: 153; SEQ ID NO: 147 and SEQ ID NO: 154; SEQ ID NO: 147 and SEQ ID NO: 155; SEQ ID NO: 148 and SEQ ID NO: 150; SEQ ID NO: 148 and SEQ ID NO: 151; SEQ ID NO: 148 and SEQ ID NO: 152; SEQ ID NO: 148 and SEQ ID NO: 153; SEQ ID NO: 148 and SEQ ID NO: 154; SEQ ID NO: 148 and SEQ ID NO: 155; SEQ ID NO: 149 and SEQ ID NO: 150; SEQ ID NO: 149 and SEQ ID NO: 151; SEQ ID NO: 149 and SEQ ID NO: 152; SEQ ID NO: 149 and SEQ ID NO: 153; SEQ ID NO: 149 and SEQ ID NO: 154; SEQ ID NO: 149 and SEQ ID NO: 155; SEQ ID NO: 156 and SEQ ID NO: 159; SEQ ID NO: 156 and SEQ ID NO: 160; SEQ ID NO: 156 and SEQ ID NO: 161; SEQ ID NO: 157 and SEQ ID NO: 159; SEQ ID NO: 157 and SEQ ID NO: 160; SEQ ID NO: 157 and SEQ ID NO: 161; SEQ ID NO: 158 and SEQ ID NO: 159; SEQ ID NO: 158 and SEQ ID NO: 160; or SEQ ID NO: 158 and SEQ ID NO: 161.

19. The multimeric antibody of claim 18, wherein the multimeric antibody is more potent than an equivalent amount of a bivalent IgG antibody comprising the VH and VL of the multimeric antibody.

20. The multimeric antibody of claim 19, which can direct complement-directed cytotoxicity (CDC) of a CD38-expressing cell at a higher potency than an equivalent amount of a bivalent IgG antibody comprising the VH and VL of the multimeric antibody.

21. The multimeric antibody of any one of claims 12 to 20, which is dimeric or tetrameric and comprises two or four bivalent IgA or IgA-like binding units and a J chain or fragment or variant thereof, wherein each binding unit comprises two IgA heavy chain constant regions or multimerizing fragments or variants thereof each comprise a Cα3 domain and an α-tail piece (αtp) domain.

22. The multimeric antibody of claim 21, wherein the IgA heavy chain constant regions or multimerizing fragments or variants thereof each further comprise a Cα1 domain, a Cα2 domain, an IgA hinge region, or any combination thereof.

23. The multimeric antibody of claim 21 or claim 22, wherein the IgA heavy chain constant regions or multimerizing fragments or variants thereof are human IgA constant regions.

24. The multimeric antibody of any one of claims 21 to 23, wherein the J-chain is a mature human J-chain comprising the amino acid sequence SEQ ID NO: 7 or a functional fragment or variant thereof.

25. The multimeric antibody of any one of claims 12 to 19, which is hexameric or pentameric and comprises five or six bivalent IgM or IgM-like binding units, wherein each binding unit comprises two IgM heavy chain constant regions or multimerizing fragments or variants thereof each comprise a Cμ4 and a μ-tail piece (μtp) domain.

26. The multimeric antibody of claim 25, wherein the IgM heavy chain constant regions or fragments or variants thereof each further comprise a Cμ1 domain, a Cμ2 domain, a Cμ3 domain, or any combination thereof.

27. The multimeric antibody of claim 25 or claim 26, wherein the IgM heavy chain constant regions or multimerizing fragments or variants thereof are human IgM constant regions.

28. The multimeric antibody of claim 27, wherein each IgM heavy chain constant region comprises the amino acid sequence SEQ ID NO: 1, SEQ ID NO: 2, or a multimerizing variant or fragment thereof.

29. The multimeric antibody of claim 27 or claim 28, wherein each IgM heavy chain constant region is a variant human IgM constant region comprising one or more single amino acid substitutions, deletions, or insertions relative to SEQ ID NO: 1 or SEQ ID NO: 2, and wherein the multimeric antibody has reduced CDC activity relative to a multimeric antibody where each IgM heavy chain constant region comprises the amino acid sequence SEQ ID NO: 1 or SEQ ID NO: 2.

30. The multimeric antibody of claim 29, wherein each variant human IgM constant region comprises an amino acid substitution corresponding to position L310 of SEQ ID NO: 1 or SEQ ID NO: 2, an amino acid substitution corresponding to position P311 of SEQ ID NO: 1 or SEQ ID NO: 2, an amino acid substitution corresponding to position P313 of SEQ ID NO: 1 or SEQ ID NO: 2, an amino acid substitution corresponding to position K315 of SEQ ID NO: 1 or SEQ ID NO: 2, or any combination thereof.

31. The multimeric antibody of any one of claims 27 to 30, wherein each IgM heavy chain constant region is a variant human IgM constant region comprising one or more single amino acid substitutions, deletions, or insertions relative to SEQ ID NO: 1 or SEQ ID NO: 2, and wherein the multimeric antibody exhibits increased serum half-life upon administration to a subject animal relative to a multimeric antibody where each IgM heavy chain constant region comprises the amino acid sequence SEQ ID NO: 1 or SEQ ID NO: 2, which is administered in the same way to the same animal species.

32. The multimeric antibody of claim 31, wherein the variant IgM heavy chain constant regions comprise half-life altering amino acid substitutions at one or more amino acid positions corresponding to amino acid E345, S401, E402, or E403 of SEQ ID NO: 1 or SEQ ID NO: 2.

33. The multimeric antibody of any one of claims 25 to 32 which is pentameric, and further comprises a J chain, or functional fragment thereof, or functional variant thereof.

34. The multimeric antibody of any one of claim 33, wherein the J-chain is a mature human J-chain comprising the amino acid sequence SEQ ID NO: 7 or a functional fragment or variant thereof.

35. The multimeric antibody of claim 33, wherein the J-chain is a variant J-chain or functional fragment thereof comprising one or more single amino acid substitutions, deletions, or insertions relative to a reference J-chain identical to the variant J-chain except for the one or more single amino acid substitutions, deletions, or insertions, and wherein the variant J-chain can affect serum half-life of the multimeric antibody; and
wherein the multimeric antibody exhibits an increased serum half-life upon administration to a subject animal relative to a reference multimeric antibody that is identical except for the one or more single amino acid substitutions, deletions, or insertions in the variant J-chain, and is administered in the same way to the same animal species.

36. The multimeric antibody of claim 35, wherein the variant J-chain comprises an amino acid substitution at an amino acid position corresponding to amino acid Y102 of the wild-type mature human J-chain of SEQ ID NO: 7.

37. The multimeric antibody of claim 36, wherein the amino acid corresponding to Y102 of SEQ ID NO: 7 is substituted with alanine (A).

38. The multimeric antibody of claim 37, wherein the J-chain comprises the amino acid sequence SEQ ID NO: 8.

39. The multimeric antibody of any one of claims 24 or 33 to 38, wherein the J-chain or functional fragment or thereof is a modified J-chain further comprising a heterologous moiety, wherein the heterologous moiety is fused or conjugated to the J-chain or functional fragment or variant thereof.

40. The multimeric antibody of claim 39, wherein the heterologous moiety is a polypeptide fused to the J-chain or functional fragment or variant thereof.

41. The multimeric antibody of claim 40, wherein the heterologous polypeptide is fused to the J-chain or functional fragment or functional variant thereof via a peptide linker comprising at least 5 amino acids, but no more than 25 amino acids.

42. The multimeric antibody of claim 40 or claim 41, wherein the heterologous polypeptide is fused to the N-terminus of the J-chain or functional fragment or variant thereof, to the C-terminus of the J-chain or functional fragment or variant thereof, or to both the N-terminus and C-terminus of the J-chain or functional fragment or variant thereof, wherein the heterologous polypeptides fused to both the N-terminus and C-terminus can be the same or different.

43. The multimeric antibody of any one of claims 40 to 42, wherein the heterologous polypeptide comprises a scFv fragment.

44. The multimeric antibody of claim 43, wherein the heterologous scFv fragment binds to CD3.

45. The multimeric antibody of claim 44, wherein the scFv fragment comprises a scFv heavy chain variable region (scFv VH) and a scFv light chain variable region (scFv VL), wherein the scFv VH comprises scFv VH complementarity-determining regions VHCDR1, VHCDR2, and VHCDR3 and the scFv VL comprises scFv VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; or SEQ ID NO: 35, SEQ ID NO: 43, SEQ ID NO: 47, SEQ ID NO: 39, SEQ ID NO: 40, and SEQ ID NO: 49.

46. The multimeric antibody of claim 45, wherein the VHCDR1, VHCDR2, VHCDR3, VLCDR1, VLCDR2, and VLCDR3 comprise, respectively, SEQ ID NO: 25, SEQ ID NO: 26, SEQ ID NO: 27, SEQ ID NO: 29, SEQ ID NO: 30, and SEQ ID NO: 31.

47. The multimeric antibody of claim 45, wherein the scFv VH and scFv VL comprise an amino acid sequence at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively.

48. The multimeric antibody of claim 47, wherein the scFv fragment comprises the scFv VH and scFv VL amino acid sequences SEQ ID NO: 24 and SEQ ID NO: 28, respectively.

49. The antibody or antigen-binding fragment or derivative thereof of any one of claims 1 to 11 or the multimeric antibody of any of claims 12 to 48, which can specifically bind to human CD38, non-human primate CD38, or human CD38 and non-human primate CD38.

50. The antibody or antigen-binding fragment or derivative thereof of claim 49 or the multimeric antibody of claim 49, wherein the non-human primate CD38 is cynomolgus monkey CD38.

51. The antibody or antigen-binding fragment or derivative thereof of any one of claims 1 to 11 or 49 to 50 or the multimeric antibody of any of claims 12 to 50, which specifically binds to CD38 with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM. 0.005 nM, or 0.001 nM; and wherein the CD38 is human CD38, cynomolgus monkey CD38, or human CD38 and cynomolgus monkey CD38.

52. The antibody or antigen-binding fragment or derivative thereof of any one of claims 1 to 11 or 49 to 51 or the multimeric antibody of any of claims 12 to 51, which is multispecific.

53. The antibody or antigen-binding fragment or derivative thereof of claim 52 or the multimeric antibody of claim 52, which is bispecific.

54. A composition comprising the antibody or antigen-binding fragment or derivative thereof of any one of claims 1 to 11 or 49 to 53 or the multimeric antibody of any of claims 12 to 53.

55. A polynucleotide comprising a nucleic acid sequence that encodes the antibody or antigen-binding fragment or derivative thereof of any one of claims 1 to 11 or 49 to 53 or the multimeric antibody of any of claims 12 to 53 or a subunit thereof.

56. A vector comprising the polynucleotide of claim 55.

57. A host cell comprising the vector of claim 56.

58. A method of producing the antibody or antigen-binding fragment or derivative thereof of any one of claims 1 to 11 or 49 to 53 or the multimeric antibody of any of claims 12 to 53, comprising culturing the host cell of claim 57, and recovering the antibody or antigen-binding fragment or derivative thereof.

59. A method of treating cancer comprising administering to a subject in need of treatment an effective amount of the antibody or antigen-binding fragment or derivative thereof of any one of claims 1 to 11 or 49 to 53 or the multimeric antibody of any of claims 12 to 53.

60. The method of claim 59, wherein the subject is human.

61. The antibody or antigen-binding fragment or derivative thereof of any one of claims 1 to 11 or 49 to 53, the multimeric antibody of any of claims 12 to 53, or the composition of claim 54 for use in treating cancer in a subject in need thereof.

62. The antibody or antigen-binding fragment or derivative thereof, the multimeric antibody, or the composition for use of claim 61, wherein the subject is human.

63. Use of the antibody or antigen-binding fragment or derivative thereof of any one of claims 1 to 11 or 49 to 53 or the multimeric antibody of any of claims 12 to 53 in treating cancer in a subject in need thereof.

64. Use of the antibody or antigen-binding fragment or derivative thereof of any one of claims 1 to 11 or 49 to 53 or the multimeric antibody of any of claims 12 to 53 in the manufacture of a medicament for treating cancer in a subject in need thereof.

65. The use of claim 63 or claim 64, wherein the subject is human.


SEQUENCE LISTING (182 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 9 - protein, 5 aa, synthetic construct
GGGGS

SEQ ID NO: 10 - protein, 10 aa, synthetic construct
GGGGSGGGGS

SEQ ID NO: 11 - protein, 15 aa, synthetic construct
GGGGSGGGGSGGGGS

SEQ ID NO: 12 - protein, 20 aa, synthetic construct
GGGGSGGGGSGGGGSGGGGS

SEQ ID NO: 13 - protein, 25 aa, synthetic construct
GGGGSGGGGSGGGGSGGGGSGGGGS

SEQ ID NO: 14 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 15 - protein, 301 aa, Macaca fascicularis
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 16 - protein, 120 aa, synthetic construct [sequence omitted]

SEQ ID NO: 17 - protein, 5 aa, synthetic construct
SYTMH

SEQ ID NO: 18 - protein, 17 aa, synthetic construct
YINPRSGYTHYNQKLKD

SEQ ID NO: 19 - protein, 11 aa, synthetic construct
SAYYDYDGFAY

SEQ ID NO: 20 - protein, 106 aa, synthetic construct [sequence omitted]

SEQ ID NO: 21 - protein, 10 aa, synthetic construct
SASSSVSYMN

SEQ ID NO: 22 - protein, 7 aa, synthetic construct
DTSKLAS

SEQ ID NO: 23 - protein, 9 aa, synthetic construct
QQWSSNPPT

SEQ ID NO: 24 - protein, 125 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 25 - protein, 5 aa, synthetic construct
TYAMN

SEQ ID NO: 26 - protein, 19 aa, synthetic construct
RIRSKYNNYATYYADSVKD

SEQ ID NO: 27 - protein, 14 aa, synthetic construct
HANFGAGYVSWFAH

SEQ ID NO: 28 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 29 - protein, 14 aa, synthetic construct
GSSTGAVTTSNYAN

SEQ ID NO: 30 - protein, 7 aa, synthetic construct
GTDKRAP

SEQ ID NO: 31 - protein, 9 aa, synthetic construct
ALWYSNHWV

SEQ ID NO: 32 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 33 - protein, 9 aa, synthetic construct
ALWYSDLWV

SEQ ID NO: 34 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 35 - protein, 5 aa, synthetic construct
DYYMH

SEQ ID NO: 36 - protein, 17 aa, synthetic construct
WIDLENANTIYDAKFQG

SEQ ID NO: 37 - protein, 10 aa, synthetic construct
DAYGRYFYDV

SEQ ID NO: 38 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 39 - protein, 17 aa, synthetic construct
KSSQSLLNARTGKNYLA

SEQ ID NO: 40 - protein, 7 aa, synthetic construct
WASTRES

SEQ ID NO: 41 - protein, 8 aa, synthetic construct
KQSYSRRT

SEQ ID NO: 42 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 43 - protein, 17 aa, synthetic construct
WIDLENANTVYDAKFQG

SEQ ID NO: 44 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 45 - protein, 8 aa, synthetic construct
KQSYFRRT

SEQ ID NO: 46 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 47 - protein, 10 aa, synthetic construct
DAYGQYFYDV

SEQ ID NO: 48 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 49 - protein, 8 aa, synthetic construct
TQSYFRRT

SEQ ID NO: 50 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 51 - protein, 14 aa, synthetic construct
HGNFGNSYVSWFAY

SEQ ID NO: 52 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 53 - protein, 14 aa, synthetic construct
RSSTGAVTTSNYAN

SEQ ID NO: 54 - protein, 7 aa, synthetic construct
GTNKRAP

SEQ ID NO: 55 - protein, 9 aa, synthetic construct
ALWYSNLWV

SEQ ID NO: 56 - protein, 118 aa, synthetic construct
QVQLQQSGAELVKPGASVKISCKASGYTFSNYWIEWVKQRPGLGLEWIGDILPGSGTTNYNEKFKGKATFTADTSSNTAYMQLSSLTSEDSAVYYCAGYGNYLWFTYWGQGTLVTVSA

SEQ ID NO: 57 - protein, 10 aa, synthetic construct
GYTFSNYWIE

SEQ ID NO: 58 - protein, 17 aa, synthetic construct
DILPGSGTTNYNEKFKG

SEQ ID NO: 59 - protein, 9 aa, synthetic construct
YGNYLWFTY

SEQ ID NO: 60 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 61 - protein, 11 aa, synthetic construct
HASQNINVWLS

SEQ ID NO: 62 - protein, 7 aa, synthetic construct
KASNLHT

SEQ ID NO: 63 - protein, 9 aa, synthetic construct
QQGQSYPYT

SEQ ID NO: 64 - protein, 122 aa, synthetic construct
QAYLQQSGAEVVRSGASVKMSCKAAGYTFTSYNMHWVKQTPGQGLEWIGYIYPGNGGTNYNQKFKGKATLTADTSSTTAYMQISSLTSEDSAVYFCARGASMITTGAWFAYWGQGTLVTVSA

SEQ ID NO: 65 - protein, 10 aa, synthetic construct
GYTFTSYNMH

SEQ ID NO: 66 - protein, 17 aa, synthetic construct
YIYPGNGGTNYNQKFKG

SEQ ID NO: 67 - protein, 13 aa, synthetic construct
GASMITTGAWFAY

SEQ ID NO: 68 - protein, 111 aa, synthetic construct
NIVLTQSPASLAVSLGQRATISCRASESLDTYGNSFMYWYQQKPGQPPKLLIYLASSLESGVPARFSGSGSRTDFTLTIDPVEADDAATYYCHQNHEDPWTFGGGTKLEIK

SEQ ID NO: 69 - protein, 15 aa, synthetic construct
RASESLDTYGNSFMY

SEQ ID NO: 70 - protein, 7 aa, synthetic construct
LASSLES

SEQ ID NO: 71 - protein, 9 aa, synthetic construct
HQNHEDPWT

SEQ ID NO: 72 - protein, 119 aa, synthetic construct
EVQLQQSGTVLARPGASVKMSCKASGYTFTRYWMHWVKQRPGQDLEWIGGFYPGNSDTRYNQKFKDKAKLTAVTSTSTAYMELSSLTNEDSAVYYCTRGDRHDEGFAYWGQGTLVTVSA

SEQ ID NO: 73 - protein, 10 aa, synthetic construct
GYTFTRYWMH

SEQ ID NO: 74 - protein, 17 aa, synthetic construct
GFYPGNSDTRYNQKFKD

SEQ ID NO: 75 - protein, 10 aa, synthetic construct
GDRHDEGFAY

SEQ ID NO: 76 - protein, 106 aa, synthetic construct
DIQMTQSPASLSASVGETVTITCRASGNIHNYLAWYQQKQGKSPQLLVYNAKTLPDGVPSRFSGSGSGTQYSLKINSLQPEDFGTYYCQNIWTNYTFGGGTKLEIK

SEQ ID NO: 77 - protein, 11 aa, synthetic construct
RASGNIHNYLA

SEQ ID NO: 78 - protein, 7 aa, synthetic construct
NAKTLPD

SEQ ID NO: 79 - protein, 8 aa, synthetic construct
QNIWTNYT

SEQ ID NO: 80 - protein, 123 aa, synthetic construct
QIQLVQSGPELKKPGETVKISCKASGYTFTNYGMNWVKQAPGEDLKWMGWLNTNTGEPTYAEEFKGRFAFSLETSASTAYLQINNLRNEDTATYFCARWYYGSSDNSYAMDYWGQGTSVTVSS

SEQ ID NO: 81 - protein, 10 aa, synthetic construct
GYTFTNYGMN

SEQ ID NO: 82 - protein, 17 aa, synthetic construct
WLNTNTGEPTYAEEFKG

SEQ ID NO: 83 - protein, 14 aa, synthetic construct
WYYGSSDNSYAMDY

SEQ ID NO: 84 - protein, 107 aa, synthetic construct
ETIVTQFPASLSMAIGEKVTIRCITSTDIDDDMNWYQQRPGEPPKLLISEGNTLRPGVPSRFSSSGYGTDFVFTFENMLSEDVADYYCLQSDSLPYTVGGGTRLEIK

SEQ ID NO: 85 - protein, 11 aa, synthetic construct
ITSTDIDDDMN

SEQ ID NO: 86 - protein, 7 aa, synthetic construct
EGNTLRP

SEQ ID NO: 87 - protein, 9 aa, synthetic construct
LQSDSLPYT

SEQ ID NO: 88 - protein, 117 aa, synthetic construct
QVQLQQSGAELVRPGTSVKMSCKAAGYTFTNHWIEWVKQRPGHGLEWIGDIFPGGGYTDYNEKFKGKATLTADTSSSTAYMQLSSLTSEDSAIYYCARGRDLAMDYWGQGTSVTVSS

SEQ ID NO: 89 - protein, 10 aa, synthetic construct
GYTFTNHWIE

SEQ ID NO: 90 - protein, 17 aa, synthetic construct
DIFPGGGYTDYNEKFKG

SEQ ID NO: 91 - protein, 8 aa, synthetic construct
GRDLAMDY

SEQ ID NO: 92 - protein, 107 aa, synthetic construct
DIQMNQSPSSLSASLGDTITITCHASQNINVWLGWYQQKPGNIPKLLIYKASNLHTGVPSRFSGSGSGTGFTLTISSLQPEDIATYYCQQGQSYPYTFGGGTKLEIK

SEQ ID NO: 93 - protein, 11 aa, synthetic construct
HASQNINVWLG

SEQ ID NO: 94 - protein, 7 aa, synthetic construct
KASNLHT

SEQ ID NO: 95 - protein, 9 aa, synthetic construct
QQGQSYPYT

SEQ ID NO: 96 - protein, 117 aa, synthetic construct
EVQLQQSGAEFVRPGASVKLSCTASGFNIKDSLMHWVKQRPEQGLEWIGWIDPEDDKTKYAPKFQDEATLTADTSSNTAYLHLSSLTSEDTAIYYCARRFLRGFAYWGQGTLVTVSA

SEQ ID NO: 97 - protein, 10 aa, synthetic construct
GFNIKDSLMH

SEQ ID NO: 98 - protein, 17 aa, synthetic construct
WIDPEDDKTKYAPKFQD

SEQ ID NO: 99 - protein, 8 aa, synthetic construct
RFLRGFAY

SEQ ID NO: 100 - protein, 113 aa, synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 101 - protein, 17 aa, synthetic construct
KSSQSLLNSGNQKNYLA

SEQ ID NO: 102 - protein, 7 aa, synthetic construct
GASTRES

SEQ ID NO: 103 - protein, 9 aa, synthetic construct
QNDHSYPYT

SEQ ID NO: 104 - protein, 116 aa, synthetic construct
QVQLQQPGPELVKPVASVKISCKTSDYAFSSSWMNWVKQRPGKGLEWIGWIYPGDGDTNYNGKFKGKATLTADKSSSTAYMQLSSLTSEDSAVYFCARGRWGMDNWGQGTSVTVSS

SEQ ID NO: 105 - protein, 10 aa, synthetic construct
DYAFSSSWMN

SEQ ID NO: 106 - protein, 17 aa, synthetic construct
WIYPGDGDTNYNGKFKG

SEQ ID NO: 107 - protein, 7 aa, synthetic construct
GRWGMDN

SEQ ID NO: 108 - protein, 107 aa, synthetic construct
DIVMTQSHKFMSTSVGDRVSITCKASQNVVTAVAWYQQKPGQSPKLLIYWASSRHTGVPDRFTGSGSGTDFTLTISNVQSEDLADYFCQQYSNYPFTFGSGTKLEIK

SEQ ID NO: 109 - protein, 11 aa, synthetic construct
KASQNVVTAVA

SEQ ID NO: 110 - protein, 7 aa, synthetic construct
WASSRHT

SEQ ID NO: 111 - protein, 9 aa, synthetic construct
QQYSNYPFT

SEQ ID NO: 112 - protein, 116 aa, synthetic construct
QVPLHQSGPELIKPGASLKISCKASDSAFNNSWMNWVRQRPGKGLEWIGWIYPGDDDTKYNGKFMGKATLTADKSSGTAYMQLSSLTSEDSAVYFCARGRWGMDYWGQGTSVTVSS

SEQ ID NO: 113 - protein, 10 aa, synthetic construct
DSAFNNSWMN

SEQ ID NO: 114 - protein, 17 aa, synthetic construct
WIYPGDDDTKYNGKFMG

SEQ ID NO: 115 - protein, 7 aa, synthetic construct
GRWGMDY

SEQ ID NO: 116 - protein, 107 aa, synthetic construct
DIVMTQFHNFMSTSVGDRVNINCKASQNVVTAVAWYQQKPGQSPKLLIYWASTRHTGVPDRFTGSGSGTDFTLTISNVQSEDLADYFCQQYSNYPFTFGSGTKLEIK

SEQ ID NO: 117 - protein, 11 aa, synthetic construct
KASQNVVTAVA

SEQ ID NO: 118 - protein, 7 aa, synthetic construct
WASTRHT

SEQ ID NO: 119 - protein, 9 aa, synthetic construct
QQYSNYPFT

SEQ ID NO: 120 - protein, 116 aa, synthetic construct
QVPLQQSGPELVKPGASVKISCKASDSAFNNSWTIWVRQRPGKGLEWIGWIYPGDDDTKYNGKFMGKATLTADKSSGTAYMQLSSLTSEDSAVYFCARGRWGMDYWGQGTSVTVSS

SEQ ID NO: 121 - protein, 10 aa, synthetic construct
DSAFNNSWTI

SEQ ID NO: 122 - protein, 17 aa, synthetic construct
WIYPGDDDTKYNGKFMG

SEQ ID NO: 123 - protein, 7 aa, synthetic construct
GRWGMDY

SEQ ID NO: 124 - protein, 107 aa, synthetic construct
DIVMTQSRKFMSTSVGDRVSISCKASQNVVTAVAWYQQKPGQSPKLLIYWASTRHTGVPDRFTGSGSGTDFTLTISNVQSEDLADYFCQQYSNYPFTFGSGTELEIK

SEQ ID NO: 125 - protein, 11 aa, synthetic construct
KASQNVVTAVA

SEQ ID NO: 126 - protein, 7 aa, synthetic construct
WASTRHT

SEQ ID NO: 127 - protein, 9 aa, synthetic construct
QQYSNYPFT

SEQ ID NO: 128 - protein, 122 aa, synthetic construct
QVQLVQSGAEVKKPGSSVKVSCKASGYTFTSYNMHWVRQAPGQGLEWMGYIYPGNGGTNYNQKFKGRVTITADESTSTAYMELSSLRSEDTAVYYCARGASMITTGAWFAYWGQGTLVTVSS

SEQ ID NO: 129 - protein, 122 aa, synthetic construct
QVQLVQSGAEVKKPGSSVKVSCKASGYTFTSYNMHWVRQAPGQGLEWMGYIYPGNGGTNYNQKFKGRVTITADTSTSTAYMELSSLRSEDTAVYYCARGASMITTGAWFAYWGQGTLVTVSS

SEQ ID NO: 130 - protein, 122 aa, synthetic construct
QVQLVQSGAEVKKPGSSVKVSCKASGYTFTSYNMHWVRQAPGQGLEWMGYIYPGSGGTNYNQKFKGRVTITADESTSTAYMELSSLRSEDTAVYYCARGASMITTGAWFAYWGQGTLVTVSS

SEQ ID NO: 131 - protein, 122 aa, synthetic construct
QVQLVQSGAEVKKPGSSVKVSCKASGYTFTSYNMHWVRQAPGQGLEWMGYIYPGNGGTSYNQKFKGRVTITADESTSTAYMELSSLRSEDTAVYYCARGASMITTGAWFAYWGQGTLVTVSS

SEQ ID NO: 132 - protein, 122 aa, synthetic construct
QVQLVQSGAEVKKPGSSVKVSCKASGYTFTSYNMHWVRQAPGQGLEWMGYIYPGSGGTSYNQKFKGRVTITADESTSTAYMELSSLRSEDTAVYYCARGASMITTGAWFAYWGQGTLVTVSS

SEQ ID NO: 133 - protein, 111 aa, synthetic construct
DIQMTQSPSSVSASVGDRVTITCRASESLDTYGNSFMYWYQQKPGKAPKLLIYLASSLESGVPSRFSGSGSGTDFTLTISSLQPEDFATYYCHQNHEDPWTFGQGTKVEIK

SEQ ID NO: 134 - protein, 17 aa, synthetic construct
YIYPGSGGTNYNQKFKG

SEQ ID NO: 135 - protein, 17 aa, synthetic construct
YIYPGSGGTSYNQKFKG

SEQ ID NO: 136 - protein, 117 aa, synthetic construct
QVQLVQSGAEVKKPGSSVKVSCKASGYTFTNHWIEWVRQAPGQGLEWMGDIFPGGGYTDYNEKFKGRVTITADESTSTAYMELSSLRSEDTAVYYCARGRDLAMDYWGQGTLVTVSS

SEQ ID NO: 137 - protein, 117 aa, synthetic construct
QVQLVQSGAEVKKPGSSVKVSCKASGYTFTNHWIEWVRQAPGQGLEWIGDIFPGGGYTDYNEKFKGRATITADESTSTAYMELSSLRSEDTAVYYCARGRDLAMDYWGQGTLVTVSS

SEQ ID NO: 138 - protein, 107 aa, synthetic construct
DIQMTQSPSSLSASVGDRVTITCHASQNINVWLGWYQQKPGKAPKLLIYKASNLHTGVPSRFSGSGSGTDFTLTISSLQPEDFATYYCQQGQSYPYTFGQGTKVEIK

SEQ ID NO: 139 - protein, 107 aa, synthetic construct
DIQMTQSPSSLSASVGDRVTITCRASQNINVWLGWYQQKPGKAPKLLIYKASNLHTGVPSRFSGSGSGTDFTLTISSLQPEDFATYYCQQGQSYPYTFGQGTKVEIK

SEQ ID NO: 140 - protein, 107 aa, synthetic construct
DIQMTQSPSSLSASVGDRVTITCSASQNINVWLGWYQQKPGKAPKLLIYKASNLHTGVPSRFSGSGSGTDFTLTISSLQPEDFATYYCQQGQSYPYTFGQGTKVEIK

SEQ ID NO: 141 - protein, 107 aa, synthetic construct
DIQMTQSPSSLSASVGDRVTITCGASQNINVWLGWYQQKPGKAPKLLIYKASNLHTGVPSRFSGSGSGTDFTLTISSLQPEDFATYYCQQGQSYPYTFGQGTKVEIK

SEQ ID NO: 142 - protein, 107 aa, synthetic construct
DIQMTQSPSSLSASVGDRVTITCEASQNINVWLGWYQQKPGKAPKLLIYKASNLHTGVPSRFSGSGSGTDFTLTISSLQPEDFATYYCQQGQSYPYTFGQGTKVEIK

SEQ ID NO: 143 - protein, 11 aa, synthetic construct
RASQNINVWLG

SEQ ID NO: 144 - protein, 11 aa, synthetic construct
SASQNINVWLG

SEQ ID NO: 145 - protein, 11 aa, synthetic construct
GASQNINVWLG

SEQ ID NO: 146 - protein, 11 aa, synthetic construct
EASQNINVWLG

SEQ ID NO: 147 - protein, 116 aa, synthetic construct
QITLKESGPTLVKPTQTLTLTCTASGFDFSRSWMNWIRQPPGKALEWLAEINPDSSTINYTTSLKDRFTISRDTSKNQVVLTMTNMDPVDTATYYCARYGNWFPYWGQGTLVTVSS

SEQ ID NO: 148 - protein, 116 aa, synthetic construct
EVQLLESGGGLVQPGGSLRLSCAASGFDFSRSWMNWVRQAPGKGLEWVSEINPDSSTINYATSLKDRFTISRDNSKNTLYLQMNSLRAEDTAVYYCARYGNWFPYWGQGTLVTVSS

SEQ ID NO: 149 - protein, 116 aa, synthetic construct
QLQLQESGPGLVKPSETLSLTCTASGFDFSRSWMNWIRQPPGKGLEWIGEINPDSSTINYATSLKDRFTISRDTSKNQLSLKLSSVTAADTAVYYCARYGNWFPYWGQGTLVTVSS

SEQ ID NO: 150 - protein, 107 aa, synthetic construct
AIQLTQSPSSLSASVGDRVTITCKASQNVDTNVAWYQQKPGKAPKALIYSASYRYSGVPSRFSGSGSGTDFTLTISSLQPEDFATYYCQQYDSYPLTFGQGTKVEIK

SEQ ID NO: 151 - protein, 107 aa, synthetic construct
DIQMTQSPSSLSASVGDRVTITCKASQNVDTNVAWYQQKPGKAPKALIYSASYRYSGVPSRFSGSGSGTDFTLTISSLQPEDFATYYCQQYDSYPLTFGQGTKVEIK

SEQ ID NO: 152 - protein, 107 aa, synthetic construct
DIQMTQSPSSLSASVGDRVTITCRASQNVDTNVAWYQQKPGKAPKALIYSASYRYSGVPSRFSGSGSGTDFTLTISSLQPEDFATYYCQQYDSYPLTFGQGTKVEIK

SEQ ID NO: 153 - protein, 107 aa, synthetic construct
DIQMTQSPSSLSASVGDRVTITCSASQNVDTNVAWYQQKPGKAPKALIYSASYRYSGVPSRFSGSGSGTDFTLTISSLQPEDFATYYCQQYDSYPLTFGQGTKVEIK

SEQ ID NO: 154 - protein, 107 aa, synthetic construct
DIQMTQSPSSLSASVGDRVTITCGASQNVDTNVAWYQQKPGKAPKALIYSASYRYSGVPSRFSGSGSGTDFTLTISSLQPEDFATYYCQQYDSYPLTFGQGTKVEIK

SEQ ID NO: 155 - protein, 107 aa, synthetic construct
DIQMTQSPSSLSASVGDRVTITCEASQNVDTNVAWYQQKPGKAPKALIYSASYRYSGVPSRFSGSGSGTDFTLTISSLQPEDFATYYCQQYDSYPLTFGQGTKVEIK

SEQ ID NO: 156 - protein, 119 aa, synthetic construct
EVQLVESGGGLVQPGGSLRLSCAVSGFSLISYGVHWVRQAPGKGLEWLGVIWRGGSTDYNAAFMSRLTISKDNSKNTVYLQMNSLRAEDTAVYYCAKTLITTGYAMDYWGQGTLVTVSS

SEQ ID NO: 157 - protein, 119 aa, synthetic construct
QVQLQESGPGLVKPSETLSLTCTVSGFSLISYGVHWVRQPPGKGLEWLGVIWRGGSTDYNAAFMSRLTISKDTSKNQVSLKLSSVTAADTAVYYCAKTLITTGYAMDYWGQGTLVTVSS

SEQ ID NO: 158 - protein, 119 aa, synthetic construct
QVQLQESGPGLVKPSDTLSLTCAVSGFSLISYGVHWVRQPPGKGLEWLGVIWRGGSTDYNAAFMSRLTMSKDTSKNQVSLKLSSVTAVDTAVYYCAKTLITTGYAMDYWGQGTLVTVSS

SEQ ID NO: 159 - protein, 106 aa, synthetic construct
DIQMTQSPSSLSASVGDRVTITCKASEDIYNRLAWYQQKPGKAPKLLISGATSLETGVPSRFSGSGSGTDYTLTISSLQPEDFATYYCQQYWSTPTFGQGTKVEIK

SEQ ID NO: 160 - protein, 106 aa, synthetic construct
DIQMTQSPSSLSASVGDRVTITCKASEDIYNRLAWYQQKPGKAPKLLISGATSLETGVPSRFSGSGSGTDYTFTISSLQPEDIATYYCQQYWSTPTFGQGTKVEIK

SEQ ID NO: 161 - protein, 106 aa, synthetic construct
EIVLTQSPDFQSVTPKEKVTITCKASEDIYNRLAWYQQKPDQSPKLLISGATSLETGVPSRFSGSGSGTDYTLTINSLEAEDAATYYCQQYWSTPTFGQGTKVEIK

SEQ ID NO: 162 - protein, 116 aa, Mus musculus
EVKLQESGGGLVQPGGSLKLSCAASGFDFSRSWMNWVRQAPGKGLEWIGEINPDSSTINYTTSLKDKFIISRDNAKNTLYLQMTKVRSEDTALYYCARYGNWFPYWGQGTLVTVSA

SEQ ID NO: 163 - protein, 107 aa, Mus musculus
DIVMTQSPKIMPTSVGDRVSVTCKASQNVDTNVAWYQQKPGQSPKALIYSASYRYSGVPDRFTGSGSGTDFTLTITNVQSEDLAEYFCQQYDSYPLTFGAGTKLDLK

SEQ ID NO: 164 - protein, 120 aa, Mus musculus
TLVQLKQSGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTSVTVSS

SEQ ID NO: 165 - protein, 105 aa, Mus musculus
IQMTQSSSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIK

SEQ ID NO: 166 - protein, 401 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 167 - protein, 400 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYP

SEQ ID NO: 168 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 169 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 170 - protein, 398 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 171 - protein, 485 aa, synthetic construct
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 214 aa, synthetic construct
DIVMTQSPSSLSASVGDRVTITCRASQNVDTWVAWYQQKPGQSPKALIYSASYRYSGVPDRFTGSGSGTDFTLTISSLQPEDFATYFCQQYDSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 445 aa, synthetic construct
EVQLVESGGGLVQPGGSLRLSCAASGFDFSRSWMNWVRQAPGKGLEWVSEINPDSSTINYATSVKGRFTISRDNSKNTLYLQMNSLRAEDTAVYYCARYGNWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 174 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 116 aa, synthetic construct; identical to SEQ ID NO: 162

SEQ ID NO: 179 - protein, 107 aa, synthetic construct; identical to SEQ ID NO: 163

SEQ ID NO: 180 - protein, 120 aa, synthetic construct; identical to SEQ ID NO: 164

SEQ ID NO: 181 - protein, 105 aa, synthetic construct; identical to SEQ ID NO: 165

SEQ ID NO: 182 - protein, 17 aa, synthetic construct
YIYPGNGGTSYNQKFKG